(19) United States
(12) Patent Application Publication
(10) Pub. No.: US 2024/0390337 A1
(43) Pub. Date: Nov. 28, 2024

(54) RELA/RSH INHIBITORS FOR THE TREATMENT OR PREVENTION OF MEDICAL BIOFILMS

(71) Applicant: DREXEL UNIVERSITY, Philadelphia, PA (US)

(72) Inventors: Donald Carl Hall, JR., Delta, PA (US); Jaroslaw E. Krol, Philadelphia, PA (US); Hai-Feng Ji, Cherry Hill, NJ (US); Garth David Ehrlich, Philadelphia, PA (US)

(21) Appl. No.: 18/694,246

(22) PCT Filed: Sep. 22, 2022
(86) PCT No.: PCT/US22/76853

§ 371 Date: Mar. 21, 2024

Related U.S. Application Data
(60) Provisional application No. 63/247,023, filed on Sep. 22, 2021.

(51) Int. Cl. A61K 31/433; A61K 31/4166; A61K 31/4178; A61K 31/4192; A61K 31/422; A61K 31/4245; A61P 31/04

(52) U.S. Cl. CPC A61K 31/433; A61K 31/4166; A61K 31/4178; A61K 31/4192; A61K 31/422; A61K 31/4245; A61P 31/04

(57) ABSTRACT

Disclosed herein are certain compounds capable of inhibiting bacterial RelA/RSH, and/or minimizing and/or inhibiting formation of bacterial biofilms, and/or compromising and/or reducing integrity of formed biofilms, and/or inhibiting the production of toxins by gram-negative bacteria. Also disclosed herein are methods of inhibiting the formation of and/or compromising and/or reducing integrity of formed biofilms, as well as methods of inhibiting production of toxins by gram-negative bacteria using the compounds disclosed herein.

### CROSS-REFERENCE TO RELATED APPLICATIONS

The present application claims priority under 35 U.S.C. § 119(e) to U.S. Provisional Patent Application No. 63/247,023, filed Sep. 22, 2021, which is incorporated herein by reference in its entirety.

### SEQUENCE LISTING

The ASCII text file named “046528-7118WO1(00990)_Seq Listing” created on Sep. 14, 2022, comprising 6.49 Kbytes, is hereby incorporated by reference in its entirety.

### BACKGROUND OF THE DISCLOSURE

Medical biofilms can be defined as an infectious organized community of bacteria that are living as a multicellular entity either on host tissue or any implantable device including arthroplasties, pace-makers, dental implants, and so forth. Bacteria have evolved a metabolic strategy to ensure their survival in these complex communities. This strategy is triggered by the “stringent response” and is nearly universally amongst bacterial species.

The keystone enzyme in triggering the process of bacterial metabolic quiescence is RelA. RelA is a highly conserved pyrophosphotransferase that is encoded by the relA gene. This enzyme is responsible for sensing amino acid starvation and is self-regulating within a bacterial biofilm. RelA catalyzes the reaction between GTP and ATP to form the “alarmones” pppGpp (guanosine pentaphosphate) and ppGpp (guanosine tetraphosphate). These molecules trigger a wholesale transcriptional change within the cell causing an upregulation of oxidative damage combating enzymes, as well as a reduction in replication machinery. Cells that have gone through these metamorphic changes are called persister cells. Persister cells, while not genetically resistant to antibiotics, can withstand up to 1000× the antibiotic concentrations of their planktonic counterparts.

There is thus an unmet need in the art for compounds that can be used to inhibit, minimize, and/or prevent medical biofilm formation. The present disclosure meets this need.

### SUMMARY

In some aspects, the present application is directed to the following non-limiting embodiments:

Methods of Treating, Ameliorating and/or Preventing Biofilm

In some aspects, the present disclosure is directed to a method of treating, ameliorating and/or preventing biofilm formation by a bacterium.

In some embodiments, the method comprises contacting the bacterium with at least one compound selected from:
(a) a compound of Formula I:

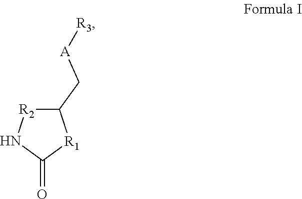

or a salt, solvate, tautomer, N-oxide, geometric isomer, stereoisomer thereof, and/or mixtures thereof, wherein:
   R₁ is —NH— or —O—,
   R₂ is —CH₂— or —C(O)—,
   A is a five member aromatic heterocyclic ring or —CH═CH—COO—*, wherein * is the bond to R₃.
   R₃ is —O—C(O)OH or

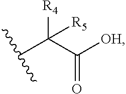

and
R₄ and R₅ are each independently H, halogen, C₁-C₆ alkyl, C₁-C₆ alkoxy, or —OH;
(b)

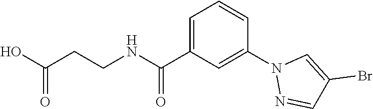 [SMILES: O=C(O)CCNC(=O)c1cccc(-n2cc(Br)cn2)c1]

3-(3-(4-bromo-1H-pyrazol-1-yl)benzamido)propanoic acid (C22), or a salt, solvate, tautomer, N-oxide, geometric isomer, and/or mixtures thereof.

In some embodiments, in Formula I, A is

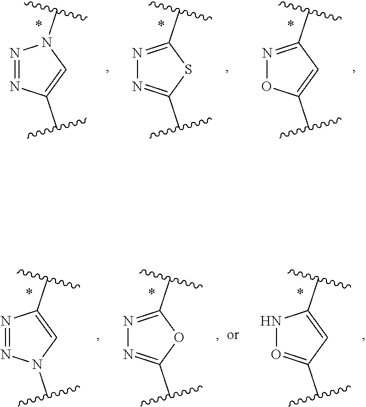

wherein * is the bond to R₃ and wherein the CH in the five-membered heterocyclyl group of A (if present) is independently optionally substituted with at least one of C₁-C₆ alkyl, C₁-C₆ alkoxy, and halogen.

In some embodiments, the compound of Formula I is at least one selected from the group consisting of:

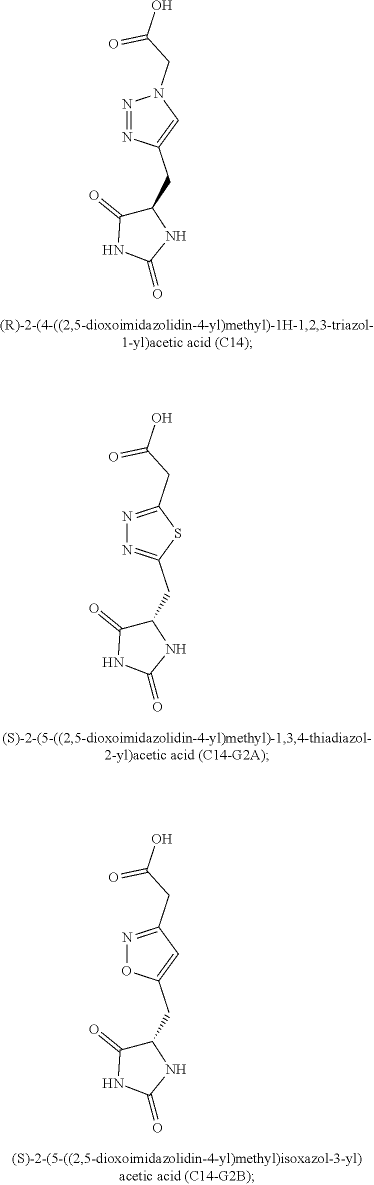 [SMILES: O=C(O)Cn1cc(C[C@H]2NC(=O)NC2=O)nn1 ; O=C(O)Cc1nnc(C[C@@H]2NC(=O)NC2=O)s1 ; O=C(O)Cc1cc(C[C@@H]2NC(=O)NC2=O)on1]

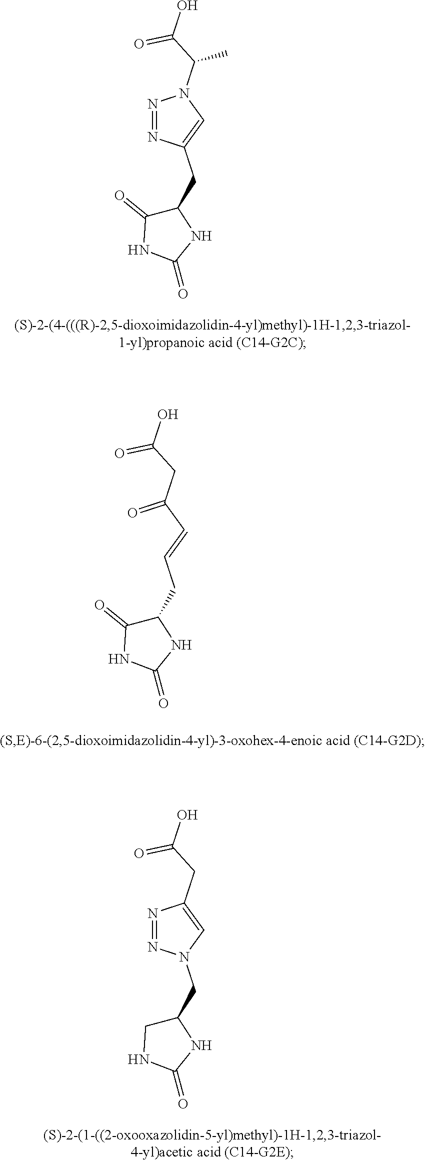 [SMILES: C[C@@H](C(=O)O)n1cc(C[C@H]2NC(=O)NC2=O)nn1 ; O=C(O)CC(=O)/C=C/C[C@@H]1NC(=O)NC1=O ; O=C(O)Cc1cn(C[C@@H]2CNC(=O)N2)nn1]

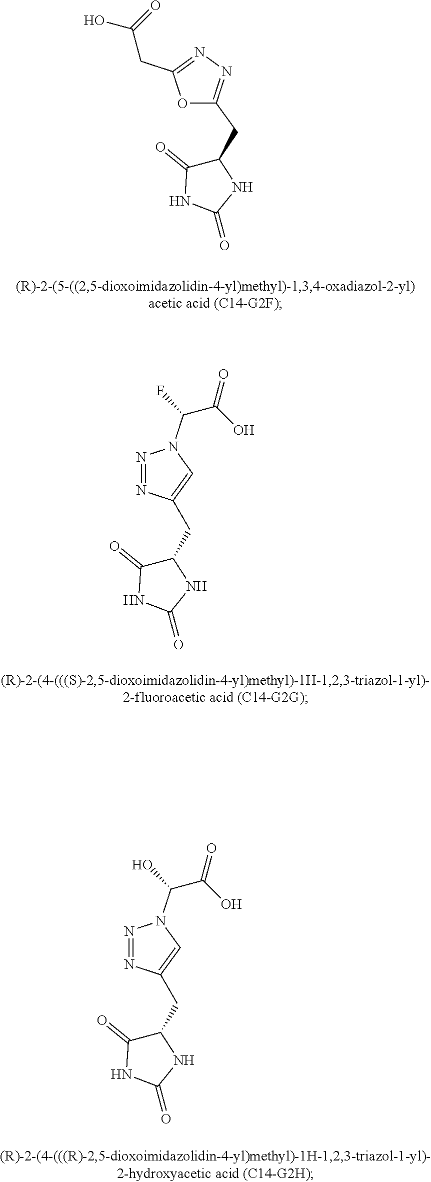 [SMILES: O=C(O)Cc1nnc(C[C@H]2NC(=O)NC2=O)o1 ; O=C1NC(=O)[C@H](Cc2cn([C@H](F)C(=O)O)nn2)N1 ; O=C1NC(=O)[C@H](Cc2cn([C@H](O)C(=O)O)nn2)N1]

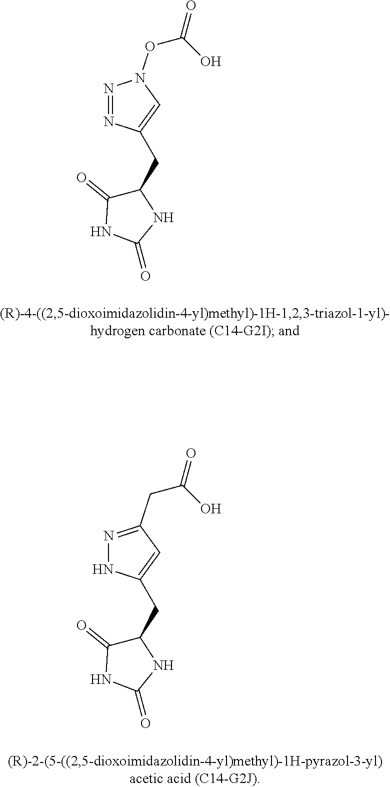 [SMILES: O=C1NC(=O)[C@@H](Cc2cn(OC(=O)O)nn2)N1 ; O=C(O)Cc1cc(C[C@H]2NC(=O)NC2=O)[nH]n1]

In some embodiments, formation of the biofilm by the bacterium is inhibited.

In some embodiments, the integrity of the biofilm already formed by the bacterium is compromised and/or reduced.

In some embodiments, the bacterium is a gram-positive bacterium or a gram-negative bacterium.

In some embodiments, the bacterium comprises a B. burgdorferi bacterium, an E. coli bacterium, an H. influenzae bacterium, an N. gonorrhoeae bacterium, a P. aeruginosa bacterium, an S. epidermidis bacterium, an S. pneumoniae bacterium, and/or an S. aureus bacterium.

In some embodiments, the method further comprises contacting the bacterium with an antibiotic for killing or inhibiting the bacterium.

In some embodiments, the biofilm is present in and/or on a subject, and the method comprises administering and/or applying an effective amount of the at least one compound to the subject.

In some embodiments, the biofilm is formed as part of a tissue-related infection in the subject or wherein the biofilm is formed in and/or on a device within the subject's body of the subject or in prolonged contact with the subject's body.

In some embodiments, the at least one compounds inhibits RelA or SpoT Homology (RSH) enzyme in the bacterium.

### Methods of Inhibiting Gram-Negative Bacterial Toxin Production

In some aspects, the present disclosure is directed to a method of inhibiting toxin production by a gram-negative bacterium.

In certain embodiments, the method comprises contacting the gram-negative bacterium with at least one compound selected from:
(a) a compound of Formula I:

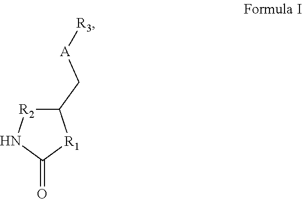

or a salt, solvate, tautomer, N-oxide, geometric isomer, stereoisomer thereof, and/or mixtures thereof, wherein:
   R₁ is —NH— or —O—,
   R₂ is —CH₂— or —C(O)—,
   A is a five member aromatic heterocyclic ring or —CH═CH—COO—*, wherein * is the bond to R₃.
   R₃ is —O—C(O)OH or

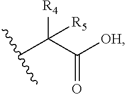

and
R₄ and R₅ are each independently H, halogen, C₁-C₆ alkyl, C₁-C₆ alkoxy, or —OH;
(b)

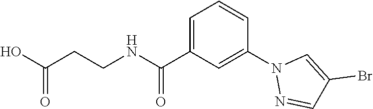 [SMILES: O=C(O)CCNC(=O)c1cccc(-n2cc(Br)cn2)c1]

3-(3-(4-bromo-1H-pyrazol-1-yl)benzamido)propanoic acid (C22), or a salt, solvate, tautomer, N-oxide, geometric isomer, and/or mixtures thereof.

In some embodiments, in Formula I, A is

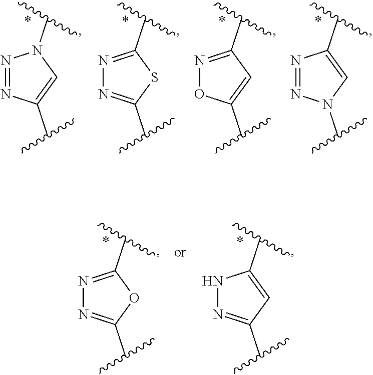 [SMILES: *c1cn(*)nn1 ; *c1nnc(*)s1 ; *c1cc(*)on1 ; *c1cn(*)nn1 ; *c1nnc(*)o1 ; *c1cc(*)[nH]n1]

wherein * is the bond to R₃ and wherein the CH in the five-membered heterocyclyl group of A (if present) is independently optionally substituted with at least one of C₁-C₆ alkyl, C₁-C₆ alkoxy, and halogen.

In some embodiments, the compound of Formula I is at least one selected from the group consisting of:

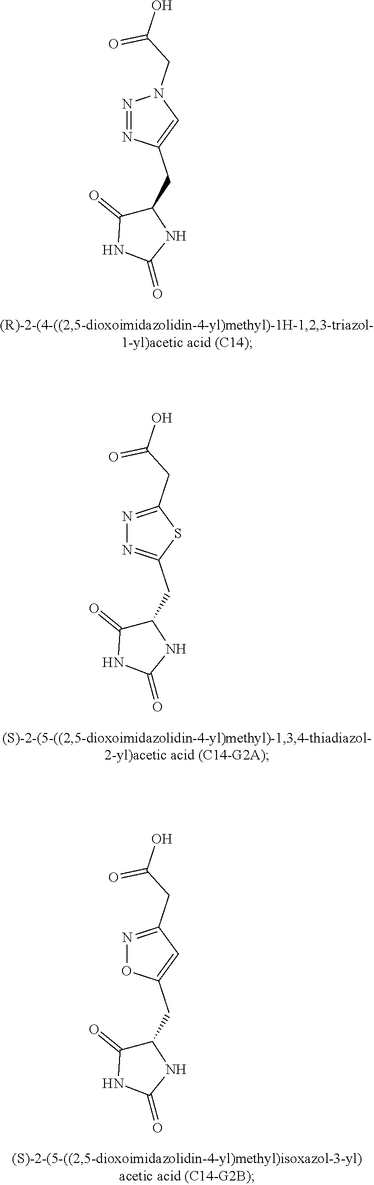 [SMILES: O=C(O)Cn1cc(C[C@H]2NC(=O)NC2=O)nn1 ; O=C(O)Cc1nnc(C[C@@H]2NC(=O)NC2=O)s1 ; O=C(O)Cc1cc(C[C@@H]2NC(=O)NC2=O)on1]

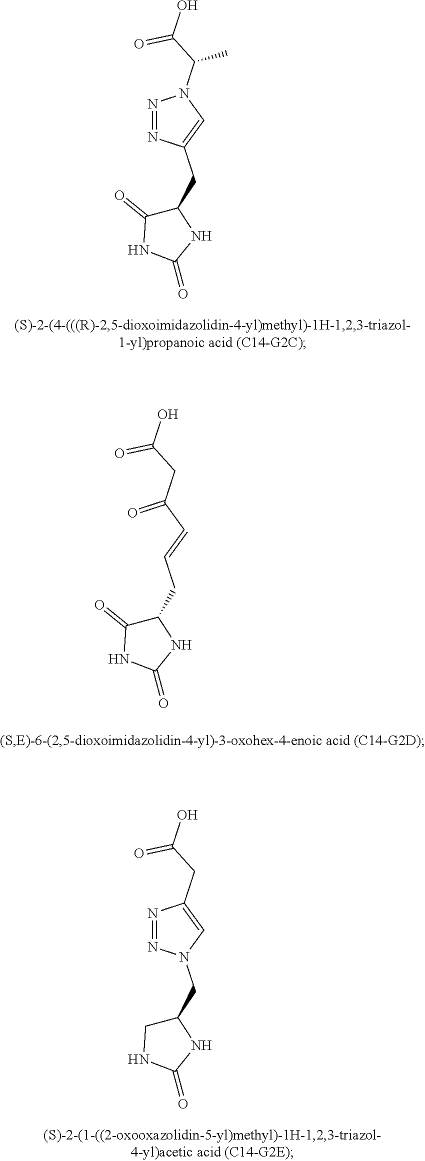 [SMILES: C[C@@H](C(=O)O)n1cc(C[C@H]2NC(=O)NC2=O)nn1 ; O=C(O)CC(=O)/C=C/C[C@@H]1NC(=O)NC1=O ; O=C(O)Cc1cn(C[C@@H]2CNC(=O)N2)nn1]

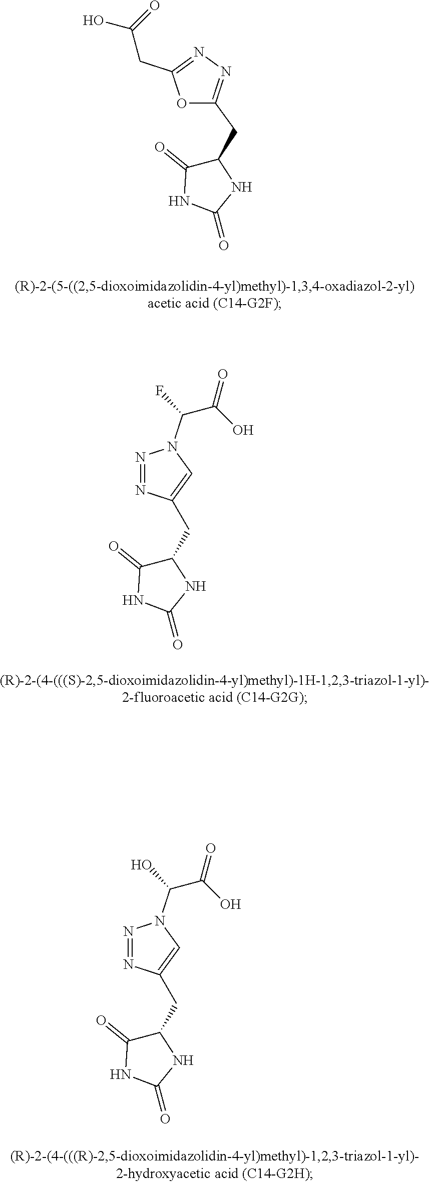 [SMILES: O=C(O)Cc1nnc(C[C@H]2NC(=O)NC2=O)o1 ; O=C1NC(=O)[C@H](Cc2cn([C@H](F)C(=O)O)nn2)N1 ; O=C1NC(=O)[C@H](Cc2cn([C@H](O)C(=O)O)nn2)N1]

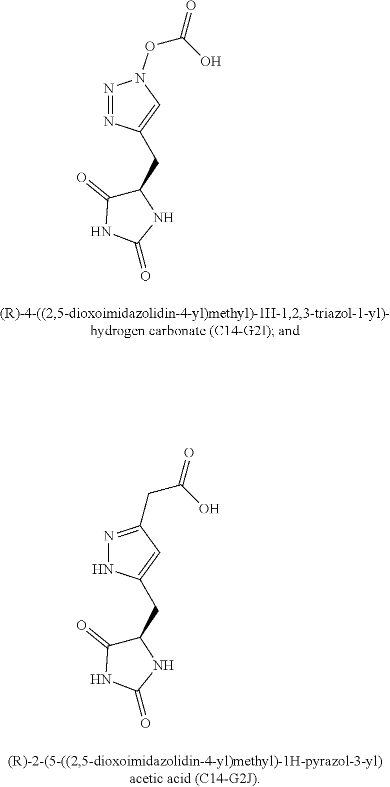 [SMILES: O=C1NC(=O)[C@@H](Cc2cn(OC(=O)O)nn2)N1 ; O=C(O)Cc1cc(C[C@H]2NC(=O)NC2=O)[nH]n1]

In some embodiments, the gram-negative bacterium comprises an E. coli bacterium, an H. influenzae bacterium, and/or a P. aeruginosa bacterium.

In some embodiments, the gram-negative bacterium is a cultured gram-negative bacterium.

In some embodiments, the gram-negative bacterium is in and/or on the body of a subject.

In some embodiments, an effective amount of the at least one compound is administered and/or applied to the subject.

In some embodiments, RelA or RSH enzyme is inhibited in the gram-negative bacterium.

### Compounds

In some aspects, the present disclosure is directed to a compound.

In some embodiments, the compound is a compound of Formula I:

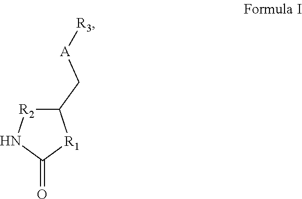

or a salt, solvate, tautomer, N-oxide, geometric isomer, stereoisomer thereof,
and/or mixtures thereof, wherein:
   R₁ is —NH— or —O—,
   R₂ is —CH₂— or —C(O)—,
   A is a five member aromatic heterocyclic ring or —CH═CH—COO—*, wherein * is the bond to R₃.
   R₃ is —O—C(O)OH or

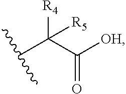

R₄ and R₅ are each independently H, halogen, C₁-C₆ alkyl, C₁-C₆ alkoxy, or —OH, and
the compound is not

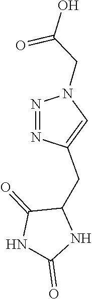 [SMILES: O=C(O)Cn1cc(CC2NC(=O)NC2=O)nn1]

2-(4-((2,5-dioxoimidazolidin-4-yl)methyl)-1H-1,2,3-triazol-1-yl)acetic acid (C14).

In some embodiments, in Formula I, A is

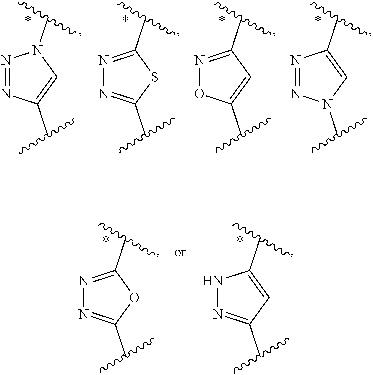 [SMILES: *c1cn(*)nn1 ; *c1nnc(*)s1 ; *c1cc(*)on1 ; *c1cn(*)nn1 ; *c1nnc(*)o1 ; *c1cc(*)[nH]n1]

wherein * is the bond to R₃ and wherein the CH in the five-membered heterocyclyl group of A (if present) is independently optionally substituted with at least one of C₁-C₆ alkyl, C₁-C₆ alkoxy, and halogen.

In some embodiments, the compound is at least one selected from the group consisting of:

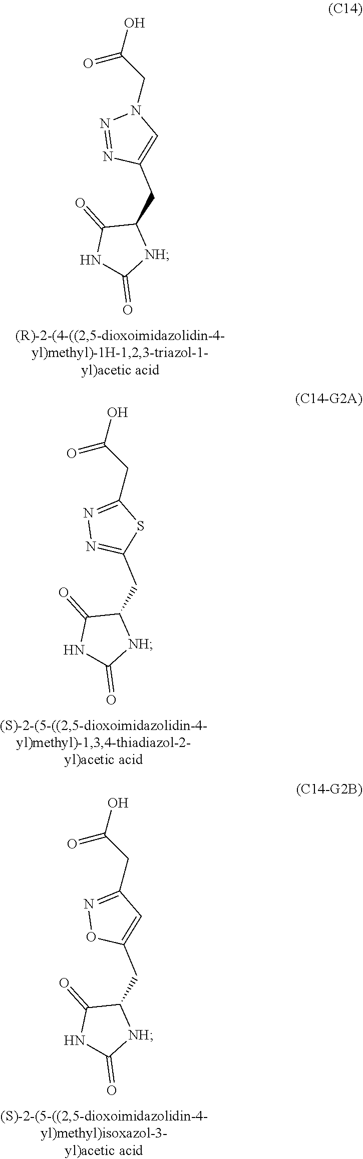 [SMILES: O=C(O)Cn1cc(C[C@H]2NC(=O)NC2=O)nn1 ; O=C(O)Cc1nnc(C[C@@H]2NC(=O)NC2=O)s1 ; O=C(O)Cc1cc(C[C@@H]2NC(=O)NC2=O)on1]

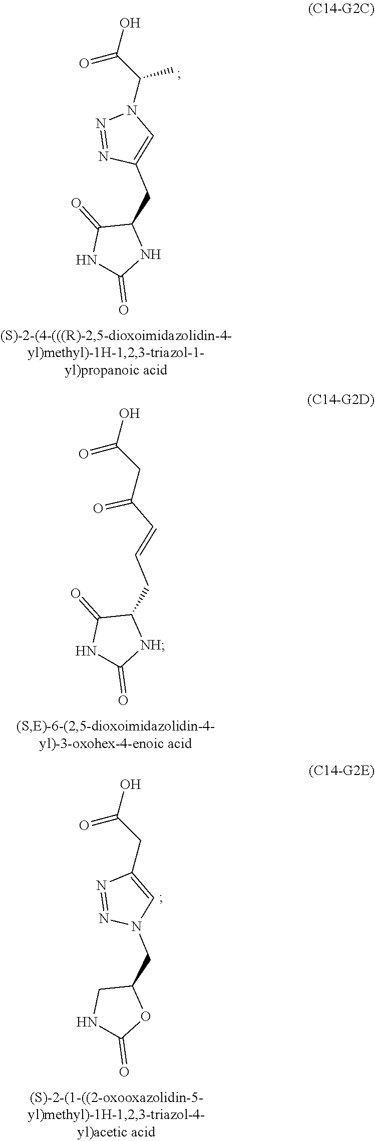 [SMILES: C[C@@H](C(=O)O)n1cc(C[C@H]2NC(=O)NC2=O)nn1 ; O=C(O)CC(=O)/C=C/C[C@@H]1NC(=O)NC1=O ; O=C(O)Cc1cn(C[C@@H]2CNC(=O)O2)nn1]

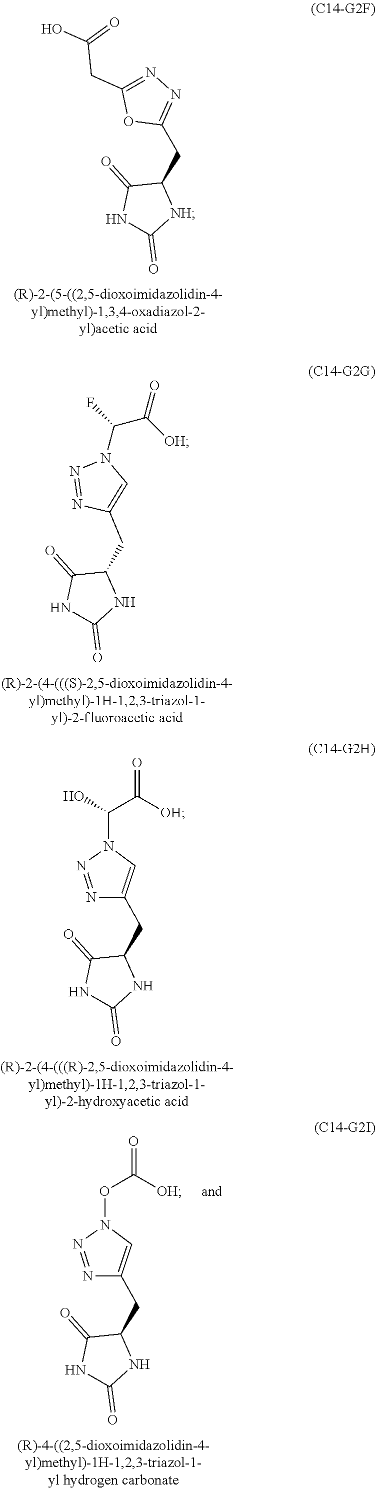 [SMILES: O=C(O)Cc1nnc(C[C@H]2NC(=O)NC2=O)o1 ; O=C1NC(=O)[C@H](Cc2cn([C@H](F)C(=O)O)nn2)N1 ; O=C1NC(=O)[C@@H](Cc2cn([C@H](O)C(=O)O)nn2)N1 ; O=C1NC(=O)[C@@H](Cc2cn(OC(=O)O)nn2)N1]

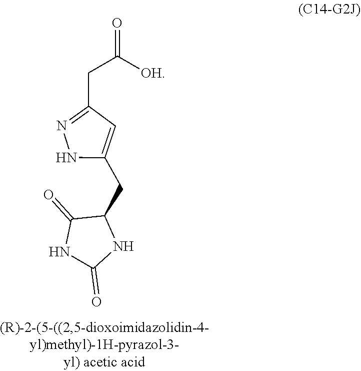 [SMILES: O=C(O)Cc1cc(C[C@H]2NC(=O)NC2=O)[nH]n1]

### BRIEF DESCRIPTION OF THE DRAWINGS

The following detailed description of exemplary embodiments will be better understood when read in conjunction with the appended drawings. For the purpose of illustrating, non-limiting embodiments are shown in the drawings. It should be understood, however, that the instant specification is not limited to the precise arrangements and instrumentalities of the embodiments shown in the drawings.

FIG. 1 is a schematic of pipeline for determination of effective RelA inhibitors according to some embodiments.

FIG. 2 illustrates certain aspects of stages of biofilm growth, in accordance with some embodiments. Stage 1-initial attachment: this is a mechanical attachment of cells to a substrate surface. This attachment may be aided by cellular structures such as pili formation. Stage 2-irreversible attachment: cells are a homogeneous layer 1-2 cells thick; they have started to produce EPS and can no longer easily become detached from the surface. Stage 3-maturation I: the biofilm starts to lose the cellular homogeneity and the biofilm can “recruit” other species of bacteria making a mixed-species biofilm. During this stage the 3D structure of the biofilm starts to take shape. Stage 4-maturation II: the biofilm becomes heterogeneous and can range in pH, oxygen concentration, nutrient concentration, and so forth. At this stage persister cells form within the biofilm. Stage 5-mature biofilm with planktonic showers: at this stage of the biofilm's development the structure releases cells into the environment to seed new biofilms.

FIGS. 3A-3D describes certain aspects of chemical makeup of EPS, in accordance with some embodiments. FIG. 3A: Structure of a 3D mixed-species biofilm. FIG. 3B: Extracellular polymers within the EPS including polysaccharides, proteins, and DNA. FIG. 3C Intermolecular forces essential to the stability of the EPS: electrostatic attractive forces (dipole moments), ionic attractive forces stabilized with cationic metals, van der Waals interactions, and hydrogen bonding; repulsive forces between similarly charged groups are also shown. FIG. 3D: Protein attachments that reinforce biofilms.

FIG. 4 is a schematic of infections caused by biofilms, in accordance with some embodiments. Left: biofilm infections related to medical devices. Right: biofilm infections related to tissues.

FIG. 5 illustrates certain aspects of RelA-mediated activation of the stringent response and its cascade events, in accordance with some embodiments. RelA activated by binding to stalled ribosome. Production of alarmones (p)ppGpp. (p)ppGpp interaction with RNA polymerase. Downregulations of nonessential cellular functions. Upregulations of stress response proteins and RNA polymerase sigma S upregulation of survival genes.

FIGS. 6A-6B illustrate structure of RelA bound to ribosome with stalled deacetylated tRNA, in accordance with some embodiments. FIG. 6A: RelA bound to the 50S and 30S subunits. FIG. 6B: Structural domains of RelA outlined: the catalytic domain (“HYD” and “SYN”) and the regulatory domains (“TGS,” “ZFD” and “RRM”).

FIG. 7 illustrate catalytic cycle of ribosome activity and production of the (p)ppGpp alarmones, in accordance with some embodiments. Normal cycle: normal conditions and translation of proteins from mRNA. Starvation cycle: amino acid starvation and the attachment of RelA to stalled ribosome and activation of RelA. RelA detaches from ribosome with deac-tRNA and enters a “semi-open” state where catalytic activity is still functional.

FIGS. 8A-8C illustrate certain aspects of the catalytic mechanism of RSH enzymes, in accordance with some embodiments. FIG. 8A: 3D arrow pushing and structural rationale of the catalytic mechanism of RelP formation of (p)ppGpp. Note that a nonhydrolyzable ATP analog was utilized to get pre-catalyzed active site structure. FIG. 8B: Post-catalytic site (p)ppGpp bound to RelP. FIG. 8C: 2D mechanism arrow pushing mechanism of the reaction of ATP and GTP to form (p)ppGpp.

FIG. 9 is a pipeline schematic of general protein ligand docking calculations, in accordance with some embodiments. Target and ligand structures are optimized and prepared for docking. A target site is determined on the protein and a grid is formed. Compounds are docked and scored and ranked based on their potential binding affinity.

FIG. 10 is a diagram of a docking grid according to some embodiments. Grid points or nodes are distributed evenly around the entire grid box. Each spacing is 2-3 Å. A probe atom is displayed.

FIG. 11 is certain results of a (p)ppGpp production assay, in accordance with some embodiments. Qualitative production of (p)ppGpp in PBS buffer under various conditions: Mg²⁺ (10 mM and 5 mM), with and without 70S ribosome.

FIGS. 12A-12D illustrate certain aspects of RelA structure and sequence homology, in accordance with some embodiments. FIG. 12A: Structural homology and alignment of known active site of S. aureus RelP with E. coli RelA; FIG. 12B: Alignment of S. aureus RelP with E. coli RelA with two essential amino acids residues shown; Fig. C: GDP interactions with the essential residues can be seen with H-bonding and π-stacking shown in dashed lines; FIG. 12D: Protein sequence alignment of selected bacterial species (both Gram-positive and Gram-negative). Arrows denote conservation of the essential residues shown in B among most bacterial species shown. The sequences shown in FIG. 12D is also shown the table below:

E. coli RelA amino acid residues 228-347:
(SEQ ID NO: 1)
YIEEFVGHLRAEMKAEGVKAEVYGRPKHIYSIWRKMQKKNLAFDE
LFDVRAVRIVAERLQDCYAALGIVHTHYRHLPDEFDDYVANPKPN
GYQSIHTVVLGPGGKTVEIQIRTKQMHEDA
S. aureus RelA amino acid residues 224-343:
(SEQ ID NO: 2)
YIETAIDRIRTEMDRMNIEGDINGRPKHIYSIYRKMMKQKKQFDQ
IFDLLAIRVIVNSINDCYAILGLVHTLWKPMPGRFKDYIAMPKON
LYQSLHTTVVGPNGDPLEIQIRTFDMHEIA
P. aeruginosa RelA amino acid residues 239-358:
(SEQ ID NO: 3)
YIANVMSQLKEALAATGVQADLSGRAKHIYSIWRKMQRKGLDFSQ
IYDVRAVRVLVPEMRDCYTALGIVHTLWRHIPKEFDDYIANPKEN
GYRSLHTAVIGPEGKVLEVQIRTHSMHEEA
S. pneumoniae RelA amino acid residues 217-335:
(SEQ ID NO: 4)
LVDEVVTKLEEYTTDRHLKGKIYGRPKHIYSIFRKMQDKRKRFEE
IYDLIAIRCILDTQSDVYAMLGYVHELWKPMPGRFKDYIANRKAN
GYQSIHTTVYGPKGPIEFQIRTKAMHEVA

FIGS. 13A-13F illustrate certain aspects of RelA GDP interactions with and without amino acid mutations, in accordance with some embodiments. FIG. 13A: 3D native RelA interaction. FIG. 13B: 3D Y/A-310 GDP interaction. FIG. 13C: 3D Y/A-319 GDP interaction.

FIG. 13D: 2D native RelA interaction. FIG. 13E: 2D Y/A-310 GDP. FIG. 13F: 2D Y/A-319 GDP.

FIG. 14 is a RelA amino acid mutation scheme according to some embodiments. Arrows indicate where the mutation was introduced in the relA expression plasmid.

FIGS. 15A-15B depict certain effects of Y/A319 and Y/A310 substitutions on RelA enzymatic activity, in accordance with some embodiments. FIG. 15A: Ratio of relative RelA activity of the induced to non-induced cells in an in vivo fluorescence assay. The induction with 1.5 μM IPTG took place at 210 min, indicated with the black triangle. FIG. 15B: In vitro pppGpp production assay. Control is [γ-32P] ATP without enzyme.

FIGS. 16A-16B are a non-limiting raw Cryo-EM structure of RelA (PDB: 5IQR), in accordance with some embodiments. FIG. 16A: RelA bound to a ribosome. FIG. 16B: optimized RelA structure with docking grid box.

FIG. 17 lists certain hit compounds for the inhibition of RelA binding score, in accordance with some embodiments. Binding score compared to the initial binding compound GTP.

FIGS. 18A-18D describe certain effects of S3-G1A and S3-GIB compounds on RelA enzymatic activity, in accordance with some embodiments. RelA (p)ppGpp production assay in vitro (FIG. 18A) and in vivo (FIG. 18B) treated with 20 μM of respective compound. Quantitative analyses of the in vitro (FIG. 18C) and in vivo (FIG. 18D) assays. * indicates statistical significance.

FIG. 19 depicts certain effects of relA and relA/spoT mutations according to some embodiments. Overnight growth curves of Wild Type E. coli and the CF1652 ΔrelA mutant.

FIG. 20 depicts certain compound effects on planktonic growth of E. coli C according to some embodiments. E. coli C treated with compounds S3-G1A and S3-GIB (30 μg·mL⁻¹).

FIG. 21 depicts certain effects of compounds on inhibition of E. coli C biofilm, in accordance with some embodiments. S3-G1A and S3-GIB effect on the formation of biofilms using the crystal violet quantification method.

FIGS. 22A-22B depict certain effects of ampicillin concentration on planktonic and biofilm growth, in accordance with some embodiments. FIG. 22A: Growth of E. coli C cells in liquid culture at various concentrations of ampicillin (OD₆₀₀). FIG. 22B: Amount of biofilm formed at various concentrations of ampicillin. (AMP #=AMP at #μg·mL⁻¹ concentration).

FIGS. 23A-23B demonstrate certain synergistic effects of compounds and ampicillin on amount of E. coli C biofilm and cell viability in biofilm, in accordance with some embodiments. FIG. 23A: Biofilm degradation utilizing hit compounds and ampicillin. A=S1-G1A (50 μM), B=S1-GIB (50 μM), Amp #=Ampicillin μg·mL⁻¹ SH=serine hydroxamate IDR=IDR 1018; FIG. 23B: AlamarBlue viability assay following combined treatment of cells with hit compounds and ampicillin shows a reduction in bacterial viability. * indicates statistical significance.

FIGS. 24A-24C show SEM images of E. coli C biofilm according to some embodiments. FIG. 24A: Untreated wild type E. coli C biofilm; FIG. 24B: E. coli C biofilm treated with 40 μg·mL⁻¹ S3-G1A; FIG. 24C: E. coli C biofilm treated with 40 μg·mL⁻¹ S3-GIB. Scale bar=1 μm.

FIG. 25 lists certain bacterial strains and plasmids utilized in the present study, in accordance with some embodiments. WT=wild type; Km=kanamycin; Cm=chloramphenicol.

FIG. 26 describes certain aspects of KNIME® and Schrödinger® pipeline for use in docking models, in accordance with some embodiments. From unprepared ligands to completely docking libraries and docking.

FIG. 27 lists certain top 40 in silico hit compounds from Enamine® HTS library, in accordance with some embodiments. Structure, assay number, and docking score (kcal/mol) are displayed in the cells.

FIG. 28 depicts certain results of the relative ppGpp inhibition assay according to some embodiments. In vitro enzymatic assay of RelA enzyme treated with compounds C1-C40. Graph shows production of ppGpp relative to the untreated control, which is set at 100% (dotted line). C14 and C22 bars=compounds picked for further analysis. Solid line indicates 15% cutoff.

FIGS. 29A-29B describes certain aspects of compounds C14 and C22, in accordance with some embodiments. FIG. 29A: Structure of Compounds C14 and C22. FIG. 29B: An example autoradiogram of an IC₅₀ measurement of C14 (0 μM-400 μM).

FIGS. 30A-30B depicts certain growth curves with Compounds C14 and C22 according to some embodiments. FIG. 30A: E. coli C; (B) P. aeruginosa (PA01).

FIG. 31 depicts a non-limiting proposed pathway of aggregation inhibition from inhibition of RelA and (p)ppGpp production, in accordance with some embodiments. Left panel: Under normal stress conditions. Right panel: Stress conditions with RelA inhibited. A.A.=amino acid.

FIG. 32 shows certain results of an aggregation assay of E. coli C with Compounds C14 and C22, in accordance with some embodiments. Top panels: Culture tubes; aggregation can be noted at the bottom of the tube in untreated sample, it is slightly absent in C22, and it is completely absent in C14. Bottom panels: microscope images of aggregations.

FIG. 33 depicts certain results of an E. coli C aggregation assay with Compounds C14 and C22 as compared to DMSO control, in accordance with some embodiments.

FIG. 34 depicts certain effects of Compounds C14 and C22 on biofilm formation in PA14, in accordance with some embodiments.

FIGS. 35A-35B depict certain effects of Compounds C14 and C22 on pyocyanin production in P. aeruginosa PA14, in accordance with some embodiments. FIG. 35A: Untreated is much greener than C22 and C14 in culture, while there is little to no green seen in C14. FIG. 35B: Chloroform extraction of pyocyanin from treated overnight cultures at various concentrations of C14.

FIGS. 36A-36B shows certain results of G. mallonella C14 toxicity assay 48 h after injection, in accordance with some embodiments. FIG. 36A: C14; FIG. 36B: DMSO/PBS control.

FIG. 37A-37E shows certain results of PA14 G. mallonella killing assay, in accordance with some embodiments. FIG. 37A: 24 h after injection of cells and DMSO control; FIG. 37B: 24 h after injection of cells and C14; FIG. 37C: 48 h after injection of cells and DMSO control; FIG. 37D: 48 h after injection of cells and C14; FIG. 37E percent survival curve of treated G. mallonella.

FIG. 38 depicts a percent survival curve of treated G. mallonella with PA14 supernatant (toxin) after overnight growth with or without C14 at 1× and 10× dilutions.

FIGS. 39A-39B depict certain aspects of the LDH cytotoxicity assay with C14 and A549 human epithelial cells, in accordance with some embodiments. FIG. 39A: Percentage of cytotoxicity relative to 100% lysis; C14 showed no cytotoxic effects. FIG. 39B: Diagram of how LDH cytotoxicity works.

FIG. 40 shows a non-limiting comparison of the purchasable analogs of C14 to C14, in accordance with some embodiments. The loss of the carboxylic acid bioisosteres resulted in loss of in vitro RelA inhibition efficacy. IC₅₀ value under each compound.

FIGS. 41A-41B illustrate certain aspects of the optimization of the compound C14, in accordance withs some embodiments. FIG. 41A: The compound is separated into 3 parts for R-group exchange. FIG. 41B: Compounds derived from C14; above each compound is its respective name and below each compound is the docking score form Glide.

FIG. 42: C14 was demonstrated to be able to almost completely shut down toxin production of the key bacterial pathogen, Pseudomonas aeruginosa, at low nanomolar concentrations. Specifically, C14 inhibits the production of toxin production by P. aeruginosa with a EC50 as low as 39.5 nM.

### DETAILED DESCRIPTION OF THE DISCLOSURE

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “organic group” as used herein refers to any carbon-containing functional group. Examples can include an oxygen-containing group such as an alkoxy group, aryloxy group, aralkyloxy group, oxo(carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀)hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

The term “alkyl” as used herein refers to straight chain and branched alkyl groups and cycloalkyl groups having from 1 to 40 carbon atoms, 1 to about 20 carbon atoms, 1 to 12 carbons or, in various embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any one of the groups listed herein, for example, amino, hydroxy, cyano, carboxy, nitro, thio, alkoxy, and halogen groups.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in various embodiments, from 2 to 8 carbon atoms. Examples include, but are not limited to vinyl, —CH═C═CCH₂, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in various embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In various embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbomyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbomyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In various embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “aralkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In various embodiments, heterocyclyl groups include 3 to about 20 ring members, whereas other such groups have 3 to about 15 ring members. A heterocyclyl group designated as a C2-heterocyclyl can be a 5-ring with two carbon atoms and three heteroatoms, a 6-ring with two carbon atoms and four heteroatoms and so forth. Likewise a C4-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C2-heteroaryl can be a 5-ring with two carbon atoms and three heteroatoms, a 6-ring with two carbon atoms and four heteroatoms and so forth. Likewise a C4-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

The term “heteroarylalkyl” as used herein refers to alkyl groups as defined herein in which a hydrogen or carbon bond of an alkyl group is replaced with a bond to a heteroaryl group as defined herein.

The term “heteroalkyl” as used herein refers to alkyl groups as defined herein in which a which a hydrogen or carbon bond of an alkyl group is replaced with at least one heteroatom such as, but not limited to, N, O, and S.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “amine” as used herein refers to primary, secondary, and tertiary amines having, e.g, the formula N (group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The terms “epoxy-functional” or “epoxy-substituted” as used herein refers to a functional group in which an oxygen atom, the epoxy substituent, is directly attached to two adjacent carbon atoms of a carbon chain or ring system. Examples of epoxy-substituted functional groups include, but are not limited to, 2,3-epoxy propyl, 3,4-epoxybutyl, 4,5-epoxypentyl, 2,3-epoxypropoxy. epoxypropoxypropyl, 2-glycidoxyethyl, 3-glycidoxypropyl, 4-glycidoxybutyl, 2-glycidoxycarbonyl)propyl, 3-(3,4-epoxycylohexyl)propyl, 2-(3,4-epoxy cyclohexyl)ethyl, 2-(2,3-epoxy cylopentyl)ethyl, 2-(4-methyl-3,4-epoxycyclohexyl)propyl, 2-(3,4-epoxy-3-methylcylohexyl)-2-methylethyl, and 5,6-epoxyhexyl.

The term “monovalent” as used herein refers to a substituent connecting via a single bond to a substituted molecule. When a substituent is monovalent, such as, for example, F or Cl, it is bonded to the atom it is substituting by a single bond.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups.

As used herein, the term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group.

The term “solvent” as used herein refers to a liquid that can dissolve a solid, liquid, or gas. Non-limiting examples of solvents are silicones, organic compounds, water, alcohols, ionic liquids, and supercritical fluids.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The term “room temperature” as used herein refers to a temperature of about 15-28° C.

The term “standard temperature and pressure” as used herein refers to 20° C. and 101 kPa.

As used herein, the term “composition” or “pharmaceutical composition” refers to a mixture of at least one compound described herein with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

As used herein, the term “efficacy” refers to the maximal effect (Emax) achieved within an assay.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds described herein include, for example, ammonium salts, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the term “pharmaceutically acceptable carrier” or “pharmaceutically acceptable excipient” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound described herein within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound(s) described herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound(s) described herein, and are physiologically acceptable to the patient. Supplementary active compounds may also be incorporated into the compositions. The “pharmaceutically acceptable carrier” may further include a pharmaceutically acceptable salt of the compound(s) described herein. Other additional ingredients that may be included in the pharmaceutical compositions used with the methods or compounds described herein are known in the art and described, for example in Remington's Pharmaceutical Sciences (Genaro, Ed., Mack Publishing Co., 1985, Easton, PA), which is incorporated herein by reference.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

As used herein, the term “potency” refers to the dose needed to produce half the maximal response (ED₅₀).

A “therapeutic” treatment is a treatment administered to a subject who exhibits signs of pathology, for the purpose of diminishing or eliminating those signs.

As used herein, the term “treatment” or “treating” is defined as the application or administration of a therapeutic agent, i.e., a compound or compounds as described herein (alone or in combination with another pharmaceutical agent), to a patient, or application or administration of a therapeutic agent to an isolated tissue or cell line from a patient (e.g., for diagnosis or ex vivo applications), who has a condition contemplated herein or a symptom of a condition contemplated herein, with the purpose to cure, heal, alleviate, relieve, alter, remedy, ameliorate, improve or affect a condition contemplated herein, or the symptoms of a condition contemplated herein. Such treatments may be specifically tailored or modified, based on knowledge obtained from the field of pharmacogenomics.

### Compounds

In one aspect, the present study, using in silico-based screening approach, discovered a family of bacterial RelA protein inhibitors. Furthermore, using several non-limiting members of the family of RelA inhibitors as examples, the present study confirmed that the family of RelA inhibitors is able to treat, ameliorate, and/or prevent bacterial biofilm formation, and/or inhibits and/or minimizes the production of toxins by gram-negative bacteria.

Accordingly, in some aspects, the present disclosure is directed to a compound for treating, ameliorating, and/or preventing bacterial biofilm formation, and/or a compound for inhibiting toxin production by a gram-negative bacterium.

In some embodiments, the biofilm is a biofilm formed by at least one bacterium.

In some embodiments, the compound inhibits and/or minimizes formation of the biofilm. In some embodiments, the compound compromises and/or reduces integrity of the biofilm which is already formed.

In some embodiments, the compound inhibits formation of the biofilm, and/or compromises and/or reduces integrity of the formed biofilm, by inhibiting RelA in at least one bacterium forming the biofilm.

In some embodiments, the compound is a compound of Formula I, or a salt, solvate, tautomer, N-oxide, geometric isomer, stereoisomer thereof, and/or mixtures thereof.

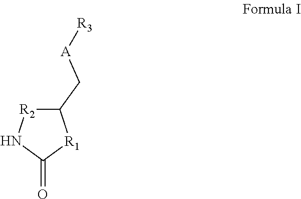

In some embodiments, in Formula I, R₁ is —NH— or —O—.

In some embodiments, R₂ is —CH₂—. In some embodiments, in Formula I, R₂ is —C(O)—.

In some embodiments, A is a five member aromatic heterocyclic ring. In some embodiments, in A is —CH═CH—COO—*, wherein * is the bond to R₃.

In some embodiments, R₃ is —O—C(O)OH. In some embodiments, R₃ is

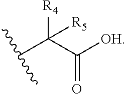

In some embodiments, R₄ and R₅ are each independently H, halogen (such as F, Cl, Br, or I), OH, C₁-C₆ alkyl, or C₁-C₆ alkoxy.

In some embodiments, R₄ is H. In some embodiments, R₄ is F. In some embodiments, R₄ is C1. In some embodiments, R₄ is Br. In some embodiments, R₄ is I. In some embodiments, R₄ is OH. In some embodiments, R₄ is C₁-C₆ alkyl. In some embodiments, R₄ is C₁-C₆ alkoxy.

In some embodiments, R₅ is H. In some embodiments, R₅ is F. In some embodiments, R₅ is C1. In some embodiments, R₅ is Br. In some embodiments, R₅ is I. In some embodiments, R₅ is OH. In some embodiments, R₅ is C₁-C₆ alkyl. In some embodiments, R₅ is C₁-C₆ alkoxy.

In some embodiments, A is

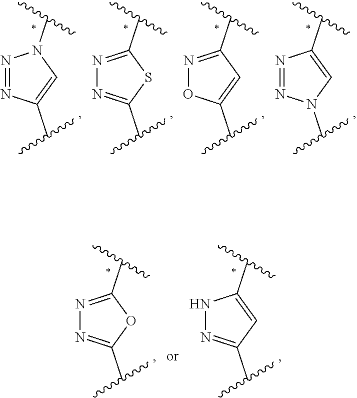

wherein * is the bond to R₃ and wherein the CH in the five-membered heterocyclyl group of A (if present) is independently optionally substituted with at least one of C₁-C₆ alkyl, C₁-C₆ alkoxy, and halogen.

In some embodiments, A is

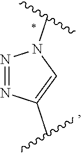 [SMILES: *c1cn(*)nn1]

wherein * is the bond to R₃ and wherein the CH in the five-membered heterocyclyl group of A is optionally substituted with at least one of C₁-C₆ alkyl, C₁-C₆ alkoxy, and halogen.

In some embodiments, A is

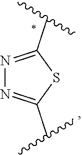 [SMILES: *c1nnc(*)s1]

wherein * is the bond to R₃ and wherein the CH in the five-membered heterocyclyl group of A is optionally substituted with at least one of C₁-C₆ alkyl, C₁-C₆ alkoxy, and halogen.

In some embodiments, A is

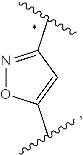 [SMILES: *c1cc(*)on1]

wherein * is the bond to R₃ and wherein the CH in the five-membered heterocyclyl group of A is optionally substituted with at least one of C₁-C₆ alkyl, C₁-C₆ alkoxy, and halogen.

In some embodiments, A is

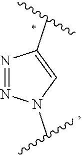 [SMILES: *c1cn(*)nn1]

wherein * is the bond to R₃ and wherein the CH in the five-membered heterocyclyl group of A is optionally substituted with at least one of C₁-C₆ alkyl, C₁-C₆ alkoxy, and halogen.

In some embodiments, A is

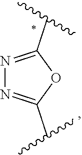 [SMILES: *c1nnc(*)o1]

wherein * is the bond to R₃.

In some embodiments, A is

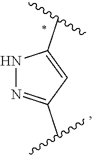 [SMILES: *c1cc(*)[nH]n1]

wherein * is the bond to R₃ and wherein the CH in the five-membered heterocyclyl group of A is optionally substituted with at least one of C₁-C₆ alkyl, C₁-C₆ alkoxy, and halogen.

In some embodiments, the compound of Formula I is:

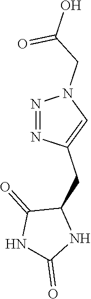 [SMILES: O=C(O)Cn1cc(C[C@H]2NC(=O)NC2=O)nn1]

(R)-2-(4-((2,5-dioxoimidazolidin-4-yl)methyl)-1H-1,2,3-triazol-1-yl)acetic acid (C14), or a salt, solvate, tautomer, N-oxide, geometric isomer, stereoisomer thereof, and/or mixtures thereof. In some embodiments, the compound is not (R)-2-(4-((2,5-dioxoimidazolidin-4-yl)methyl)-1H-1,2,3-triazol-1-yl)acetic acid (C14), or a salt, solvate, tautomer, N-oxide, geometric isomer, stereoisomer thereof, and/or mixtures thereof.

In some embodiments, the compound of Formula I is:

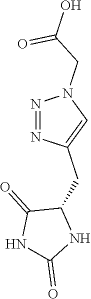 [SMILES: O=C(O)Cn1cc(C[C@@H]2NC(=O)NC2=O)nn1]

(S)-2-(4-((2,5-dioxoimidazolidin-4-yl)methyl)-1H-1,2,3-triazol-1-yl)acetic acid (C14), or a salt, solvate, tautomer, N-oxide, geometric isomer, stereoisomer thereof, and/or mixtures thereof. In some embodiments, the compound is not (S)-2-(4-((2,5-dioxoimidazolidin-4-yl)methyl)-1H-1,2,3-triazol-1-yl)acetic acid (C14), or a salt, solvate, tautomer, N-oxide, geometric isomer, stereoisomer thereof, and/or mixtures thereof.

In some embodiments, the compound of Formula I is:

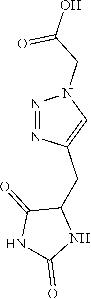 [SMILES: O=C(O)Cn1cc(CC2NC(=O)NC2=O)nn1]

2-(4-((2,5-dioxoimidazolidin-4-yl)methyl)-1H-1,2,3-triazol-1-yl)acetic acid (C14), or a salt, solvate, tautomer, N-oxide, geometric isomer, stereoisomer thereof, and/or mixtures thereof. In some embodiments, the compound is not 2-(4-((2,5-dioxoimidazolidin-4-yl)methyl)-1H-1,2,3-triazol-1-yl)acetic acid (C14), or a salt, solvate, tautomer, N-oxide, geometric isomer, stereoisomer thereof, and/or mixtures thereof.

In some embodiments, the compound of Formula I is:

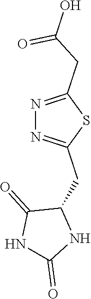 [SMILES: O=C(O)Cc1nnc(C[C@@H]2NC(=O)NC2=O)s1]

(S)-2-(5-((2,5-dioxoimidazolidin-4-yl)methyl)-1,3,4-thiadiazol-2-yl)acetic acid (C14-G2A), or a salt, solvate, tautomer, N-oxide, geometric isomer, stereoisomer thereof, and/or mixtures thereof. In some embodiments, the compound is not (S)-2-(5-((2,5-dioxoimidazolidin-4-yl)methyl)-1,3,4-thiadiazol-2-yl)acetic acid (C14-G2A), or a salt, solvate, tautomer, N-oxide, geometric isomer, stereoisomer thereof, and/or mixtures thereof.

In some embodiments, the compound of Formula I is:

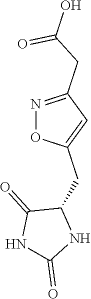 [SMILES: O=C(O)Cc1cc(C[C@@H]2NC(=O)NC2=O)on1]

(S)-2-(5-((2,5-dioxoimidazolidin-4-yl)methyl)isoxazol-3-yl)acetic acid (C14-G2B), or a salt, solvate, tautomer, N-oxide, geometric isomer, stereoisomer thereof, and/or mixtures thereof. In some embodiments, the compound is not (S)-2-(5-((2,5-dioxoimidazolidin-4-yl)methyl)isoxazol-3-yl)acetic acid (C14-G2B), or a salt, solvate, tautomer, N-oxide, geometric isomer, stereoisomer thereof, and/or mixtures thereof.

In some embodiments, the compound of Formula I is:

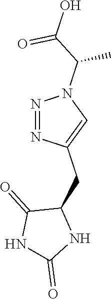 [SMILES: C[C@@H](C(=O)O)n1cc(C[C@H]2NC(=O)NC2=O)nn1]

(S)-2-(4-(((R)-2,5-dioxoimidazolidin-4-yl)methyl)-1H-1,2,3-triazol-1-yl)propanoic acid (C14-G2C), or a salt, solvate, tautomer, N-oxide, geometric isomer, stereoisomer thereof, and/or mixtures thereof. In some embodiments, the compound is not (S)-2-(4-(((R)-2,5-dioxoimidazolidin-4-yl)methyl)-1H-1,2,3-triazol-1-yl)propanoic acid (C14-G2C), or a salt, solvate, tautomer, N-oxide, geometric isomer, stereoisomer thereof, and/or mixtures thereof.

In some embodiments, the compound of Formula I is:

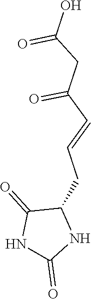 [SMILES: O=C(O)CC(=O)/C=C/C[C@@H]1NC(=O)NC1=O]

(S,E)-6-(2,5-dioxoimidazolidin-4-yl)-3-oxohex-4-enoic acid (C14-G2D), or a salt, solvate, tautomer, N-oxide, geometric isomer, stereoisomer thereof, and/or mixtures thereof. In some embodiments, the compound is not (S,E)-6-(2,5-dioxoimidazolidin-4-yl)-3-oxohex-4-enoic acid (C14-G2D), or a salt, solvate, tautomer, N-oxide, geometric isomer, stereoisomer thereof, and/or mixtures thereof.

In some embodiments, the compound of Formula I is:

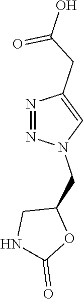 [SMILES: O=C(O)Cc1cn(C[C@@H]2CNC(=O)O2)nn1]

(S)-2-(1-((2-oxooxazolidin-5-yl)methyl)-1H-1,2,3-triazol-4-yl)acetic acid (C14-G2E), or a salt, solvate, tautomer, N-oxide, geometric isomer, stereoisomer thereof, and/or mixtures thereof. In some embodiments, the compound is not (S)-2-(1-((2-oxooxazolidin-5-yl)methyl)-1H-1,2,3-triazol-4-yl)acetic acid (C14-G2E), or a salt, solvate, tautomer, N-oxide, geometric isomer, stereoisomer thereof, and/or mixtures thereof

In some embodiments, the compound of Formula I is:

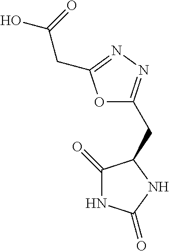 [SMILES: O=C(O)Cc1nnc(C[C@H]2NC(=O)NC2=O)o1]

(R)-2-(5-((2,5-dioxoimidazolidin-4-yl)methyl)-1,3,4-oxadiazol-2-yl)acetic acid (C14-G2F), or a salt, solvate, tautomer, N-oxide, geometric isomer, stereoisomer thereof, and/or mixtures thereof. In some embodiments, the compound is not (R)-2-(5-((2,5-dioxoimidazolidin-4-yl)methyl)-1,3,4-oxadiazol-2-yl)acetic acid (C14-G2F), or a salt, solvate, tautomer, N-oxide, geometric isomer, stereoisomer thereof, and/or mixtures thereof

In some embodiments, the compound of Formula I is:

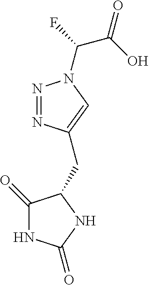 [SMILES: O=C1NC(=O)[C@H](Cc2cn([C@H](F)C(=O)O)nn2)N1]

(R)-2-(4-(((S)-2,5-dioxoimidazolidin-4-yl)methyl)-1H-1,2,3-triazol-1-yl)-2-fluoroacetic acid (C14-G2G), or a salt, solvate, tautomer, N-oxide, geometric isomer, stereoisomer thereof, and/or mixtures thereof. In some embodiments, the compound is not (R)-2-(4-(((S)-2,5-dioxoimidazolidin-4-yl)methyl)-1H-1,2,3-triazol-1-yl)-2-fluoroacetic acid (C14-G2G), or a salt, solvate, tautomer, N-oxide, geometric isomer, stereoisomer thereof, and/or mixtures thereof

In some embodiments, the compound of Formula I is:

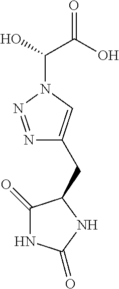 [SMILES: O=C1NC(=O)[C@@H](Cc2cn([C@H](O)C(=O)O)nn2)N1]

(R)-2-(4-(((R)-2,5-dioxoimidazolidin-4-yl)methyl)-1H-1,2,3-triazol-1-yl)-2-hydroxyacetic acid (C14-G2H), or a salt, solvate, tautomer, N-oxide, geometric isomer, stereoisomer thereof, and/or mixtures thereof. In some embodiments, the compound is not (R)-2-(4-(((R)-2,5-dioxoimidazolidin-4-yl)methyl)-1H-1,2,3-triazol-1-yl)-2-hydroxyacetic acid (C14-G2H), or a salt, solvate, tautomer, N-oxide, geometric isomer, stereoisomer thereof, and/or mixtures thereof.

In some embodiments, the compound of Formula I is:

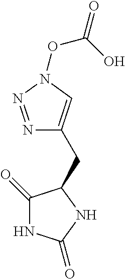 [SMILES: O=C1NC(=O)[C@@H](Cc2cn(OC(=O)O)nn2)N1]

(R)-4-((2,5-dioxoimidazolidin-4-yl)methyl)-1H-1,2,3-triazol-1-yl hydrogen carbonate (C14-G2I), or a salt, solvate, tautomer, N-oxide, geometric isomer, stereoisomer thereof, and/or mixtures thereof. In some embodiments, the compound is not (R)-4-((2,5-dioxoimidazolidin-4-yl)methyl)-1H-1,2,3-triazol-1-yl hydrogen carbonate (C14-G2I), or a salt, solvate, tautomer, N-oxide, geometric isomer, stereoisomer thereof, and/or mixtures thereof.

In some embodiments, the compound of Formula I is:

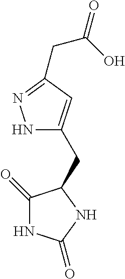 [SMILES: O=C(O)Cc1cc(C[C@H]2NC(=O)NC2=O)[nH]n1]

(R)-2-(5-((2,5-dioxoimidazolidin-4-yl)methyl)-1H-pyrazol-3-yl)acetic acid (C14-G2J), or a salt, solvate, tautomer, N-oxide, geometric isomer, stereoisomer thereof, and/or mixtures thereof. In some embodiments, the compound is not (R)-2-(5-((2,5-dioxoimidazolidin-4-yl)methyl)-1H-pyrazol-3-yl)acetic acid (C14-G2J), or a salt, solvate, tautomer, N-oxide, geometric isomer, stereoisomer thereof, and/or mixtures thereof.

In some embodiments, the compound is

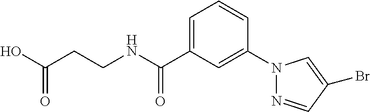 [SMILES: O=C(O)CCNC(=O)c1cccc(-n2cc(Br)cn2)c1]

3-(3-(4-bromo-1H-pyrazol-1-yl)benzamido)propanoic acid (C22), or a salt, solvate, tautomer, N-oxide, geometric isomer, stereoisomer thereof, and/or mixtures thereof. In some embodiments, the compound is not 3-(3-(4-bromo-1H-pyrazol-1-yl)benzamido)propanoic acid (C22), or a salt, solvate, tautomer, N-oxide, geometric isomer, stereoisomer thereof, and/or mixtures thereof.

The compounds described herein can possess one or more stereocenters, and each stereocenter can exist independently in either the (R) or (S) configuration. In certain embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound(s) described herein, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol. In other embodiments, the compounds described herein exist in unsolvated form.

In certain embodiments, the compound(s) described herein can exist as tautomers. All tautomers are included within the scope of the compounds presented herein.

In certain embodiments, compounds described herein are prepared as prodrugs. A “prodrug” refers to an agent that is converted into the parent drug in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain embodiments, sites on, for example, the aromatic ring portion of compound(s) described herein are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S In certain embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000,2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

### Synthesis

In some embodiments, the compound of the instant specification can be prepared by a synthesis method the same as or similar to those as described in the “Example 3-4: Experimental” section.

The instant specification further provides methods of preparing the compound of the instant specification. Compounds of the instant specification can be prepared in accordance with the procedures outlined herein, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field.

It is appreciated that where typical or preferred process conditions (i.e., reaction temperatures, times, mole ratios of reactants, solvents, pressures, and so forth) are given, other process conditions can also be used unless otherwise stated. Optimum reaction conditions can vary with the particular reactants or solvent used, but such conditions can be determined by one skilled in the art by routine optimization procedures. Those skilled in the art of organic synthesis will recognize that the nature and order of the synthetic steps presented can be varied for the purpose of optimizing the formation of the compounds described herein.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatography such as high-performance liquid chromatograpy (HPLC), gas chromatography (GC), gel-permeation chromatography (GPC), or thin layer chromatography (TLC).

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4th Ed., (Wiley 1992); Carey & Sundberg, Advanced Organic Chemistry 4th Ed., Vols. A and B (Plenum 2000,2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In some embodiments, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In other embodiments, each protective group is removable by a different means. Protective groups that are cleaved under totally disparate reaction conditions fulfill the requirement of differential removal.

In some embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In some embodiments, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from.

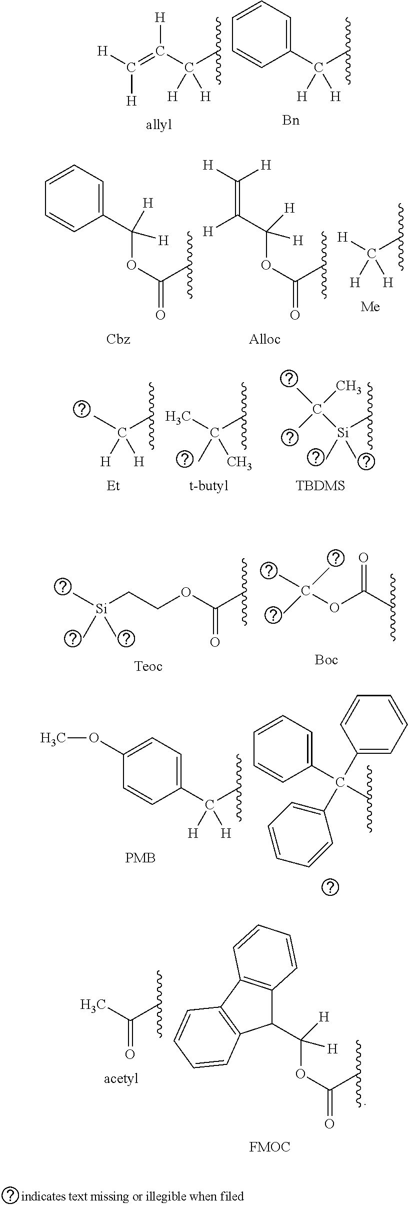

Other protecting groups, plus a detailed description of techniques applicable to the creation of protecting groups and their removal are described in Greene & Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure.

In some embodiments, a compound of the instant specification can be prepared, for example, according to the illustrative synthetic methods outlined herein.

Methods of Treating, Ameliorating and/or Preventing Bioflilm

The present study discovered that certain bacterial RelA protein inhibitors are able to inhibit and/or minimize formation of the biofilms by bacteria, and/or compromises and/or reduces integrity of bacterial biofilms that are already formed. As used herein, the phase “to compromises and/or reduces integrity of bacterial biofilms that are already formed” means to render biofilms more susceptible to biofilm degradation through (bio)chemical treatments (such as by antibiotic treatment, such as but not limited to, by heavy metal treatment) and/or, in the case of biofilms in the body of a subject, more susceptible to attack from host immune systems.

Accordingly, in some aspects, the present disclosure is directed to a method of treating, ameliorating and/or preventing biofilm formation by a bacterium.

In some embodiments, the method prevents the biofilm by inhibiting formation of the biofilm by a bacterium.

In some embodiments, the method treats and/or ameliorates the biofilm by compromising and/or reducing integrity of the biofilm already formed by a bacterium.

In some embodiments, the bacterium is a gram-positive bacterium. In some embodiments, the bacterium is a gram-negative bacterium. In some embodiments, the bacterium includes a B. burgdorferi bacterium, an E. coli bacterium, an H. influenzae bacterium, an N. gonorrhoeae bacterium, a P. aeruginosa bacterium, an S. epidermidis bacterium, an S. pneumoniae bacterium, an S. aureus bacterium, or the like.

In some embodiments, the method includes contacting the bacterium with a compound similar to or the same as the compound as described elsewhere herein, such as in the “Compound” section.

In some embodiments, the method further includes contacting the bacterium forming the biofilm with an antibiotic for killing the bacterium or inhibiting a growth thereof. One of ordinary skill in the art would be able to choose antibiotics based on, for example, the species, strains and/or gram staining profile of the bacterium, or the location of the biofilm (inside the body of a subject vs. exposed in the environment, and so forth) In some embodiments, the method further includes contacting the bacterium forming the biofilm with a metal element having toxicity to the bacterium. Examples of the metals having toxicity to bacteria include arsenic, cadmium, chromium, cobalt, copper, lead, mercury, nickel, silver, zinc, and the like.

In some embodiments, the biofilm is formed in a subject. In some embodiments, the biofilm is causing a tissue-related infection in the subject. Examples of tissue-related infections involving biofilms including chronic otitis media (COM), chronic sinusitis, chronic tonsillitis, dental plaque, chronic laryngitis, endocarditis, lung infection in cystic fibrosis, kidney stones, biliary tract infections, urinary tract infections, osteomyelitis, chronic wounds, and the like. In some embodiments, the biofilm is formed on a device in the body of the subject or in prolonged contact with the body of the subject. Examples of devices that biofilm can attach to include ventricular derivations, contact lenses, endotracheal tubes, central vascular catheters, prosthetic cardiac valves, pacemakers, vascular grafts, tissue fillers, breast implants, peripheral vascular catheters, urinary catheters, orthopedic implants, prosthetic joints, and the like.

In some embodiments, the method treats, ameliorates and/or prevents formation of a biofilm in a subject in need thereof. In some embodiments, the method includes administering and/or applying to the subject any compound herein. In some embodiments, the method further includes administering and/or applying to the subject an antibiotic capable of killing or inhibiting the bacterium.

In some embodiments, the biofilm is not in the body of a subject or on a device in the body of the subject or in prolonged contact with the body of the subject. In some embodiments, the biofilm is formed on an article exposed to the environment. In some embodiments, the method includes contacting the article with any compound herein. In some embodiments, the method further includes contact the article with a metal element having toxicity to the bacterium.

In some embodiments, the method inhibits the formation of the biofilm or reverse the formed biofilm by inhibiting RelA or RSH enzyme the bacterium forming the biofilm.

### Methods of Inhibiting Gram-Negative Bacterial Toxin Production

The present study discovered that certain bacterial RelA/RSH protein inhibitors are able to inhibit the production of toxins by gram-negative bacteria, such as but not limited to P. aeruginosa.

Accordingly, in some aspects, the present disclosure is directed to a method of inhibiting toxin production by a gram-negative bacterium.

In some embodiments, the gram-negative bacterium includes an E. coli bacterium, an H. influenzae bacterium, a P. aeruginosa bacterium, or the like.

In some embodiments, the method includes contacting the gram-negative bacterium with a compound similar to or the same as those described elsewhere herein, such as in the “Compound” section.

In some embodiments, the gram-negative bacterium is a cultured gram-negative bacterium.

In some embodiments, the gram-negative bacterium is in the body of a subject. In some embodiments, the method includes administering to the subject an effective amount of any compound herein.

In some embodiments, the method inhibits toxin production by the gram-negative bacterium by inhibiting RelA or RSH enzyme in the gram-negative bacterium.

### Compositions

In some embodiments, the instant specification is directed to a composition including a compound disclosed herein, and/or a salt, solvate, tautomer, N-oxide, geometric isomer, stereoisomer thereof, and/or any mixture thereof. The composition as well as the formulation thereof is detailed in this section, as well as in the “Pharmaceutical Compositions and Formulations” section.

The compositions containing the compound(s) described herein include a pharmaceutical composition comprising at least one compound as described herein and at least one pharmaceutically acceptable carrier. In one embodiment, a pharmaceutical composition includes at least one compound of the disclosure and at least one pharmaceutically acceptable excipient or carrier.

In certain embodiments, the composition is formulated for an administration route such as oral or parenteral, for example, transdermal, transmucosal (e.g., sublingual, lingual, (trans)buccal, (trans)urethral, vaginal (e.g., trans- and perivaginally), (intra)nasal and (trans)rectal, intravesical, intrapulmonary, intraduodenal, intragastrical, intrathecal, subcutaneous, intramuscular, intradermal, intra-arterial, intravenous, intrabronchial, inhalation, and topical administration.

### Pharmaceutical Compositions and Formulations

The instant specification provides pharmaceutical compositions comprising at least one compound of the instant specification or a salt or solvate thereof, which are useful to practice methods of the instant specification. Such a pharmaceutical composition may consist of at least one compound of the instant specification or a salt or solvate thereof, in a form suitable for administration to a subject, or the pharmaceutical composition may comprise at least one compound of the instant specification or a salt or solvate thereof, and one or more pharmaceutically acceptable carriers, one or more additional ingredients, or any combinations of these. At least one compound of the instant specification may be present in the pharmaceutical composition in the form of a physiologically acceptable salt, such as in combination with a physiologically acceptable cation or anion, as is well known in the art.

In some embodiments, the pharmaceutical compositions useful for practicing the method of the instant specification may be administered to deliver a dose of between 1 ng/kg/day and 100 mg/kg/day. In other embodiments, the pharmaceutical compositions useful for practicing the instant specification may be administered to deliver a dose of between 1 ng/kg/day and 1,000 mg/kg/day.

The relative amounts of the active ingredient, the pharmaceutically acceptable carrier, and any additional ingredients in a pharmaceutical composition of the instant specification will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the instant specification may be suitably developed for nasal, inhalational, oral, rectal, vaginal, pleural, peritoneal, parenteral, topical, transdermal, pulmonary, intranasal, buccal, ophthalmic, epidural, intrathecal, intravenous, or another route of administration. A composition useful within the methods of the instant specification may be directly administered to the brain, the brainstem, or any other part of the central nervous system of a mammal or bird. Other contemplated formulations include projected nanoparticles, microspheres, liposomal preparations, coated particles, polymer conjugates, resealed erythrocytes containing the active ingredient, and immunologically-based formulations.

In some embodiments, the compositions of the instant specification are part of a pharmaceutical matrix, which allows for manipulation of insoluble materials and improvement of the bioavailability thereof, development of controlled or sustained release products, and generation of homogeneous compositions. By way of example, a pharmaceutical matrix may be prepared using hot melt extrusion, solid solutions, solid dispersions, size reduction technologies, molecular complexes (e.g., cyclodextrins, and others), microparticulate, and particle and formulation coating processes. Amorphous or crystalline phases may be used in such processes.

The route(s) of administration will be readily apparent to the skilled artisan and will depend upon any number of factors including the type and severity of the disease being treated, the type and age of the veterinary or human patient being treated, and the like.

The formulations of the pharmaceutical compositions described herein may be prepared by any method known or hereafter developed in the art of pharmacology and pharmaceutics. In general, such preparatory methods include the step of bringing the active ingredient into association with a carrier or one or more other accessory ingredients, and then, if necessary or desirable, shaping or packaging the product into a desired single-dose or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the instant specification is contemplated include, but are not limited to, humans and other primates, mammals including commercially relevant mammals such as cattle, pigs, horses, sheep, cats, and dogs.

In some embodiments, the compositions of the instant specification are formulated using one or more pharmaceutically acceptable excipients or carriers. In some embodiments, the pharmaceutical compositions of the instant specification comprise a therapeutically effective amount of at least one compound of the instant specification and a pharmaceutically acceptable carrier. Pharmaceutically acceptable carriers, which are useful, include, but are not limited to, glycerol, water, saline, ethanol, recombinant human albumin (e.g., RECOMBUMIN®), solubilized gelatins (e.g., GELOFUSINE®), and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), recombinant human albumin, solubilized gelatins, suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, isotonic agents, for example, sugars, sodium chloride, or polyalcohols such as mannitol and sorbitol, are included in the composition. Prolonged absorption of the injectable compositions may be brought about by including in the composition an agent that delays absorption, for example, aluminum monostearate or gelatin.

Formulations may be employed in admixtures with conventional excipients, i.e., pharmaceutically acceptable organic or inorganic carrier substances suitable for oral, parenteral, nasal, inhalational, intravenous, subcutaneous, transdermal enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring, and/or fragrance-conferring substances and the like. They may also be combined where desired with other active agents, e.g., other analgesic, anxiolytics or hypnotic agents. As used herein, “additional ingredients” include, but are not limited to, one or more ingredients that may be used as a pharmaceutical carrier.

The composition of the instant specification may comprise a preservative from about 0.005% to 2.0% by total weight of the composition. The preservative is used to prevent spoilage in the case of exposure to contaminants in the environment. Examples of preservatives useful in accordance with the instant specification include but are not limited to those selected from the group consisting of benzyl alcohol, sorbic acid, parabens, imidurea and any combinations thereof. One such preservative is a combination of about 0.5% to 2.0% benzyl alcohol and 0.05-0.5% sorbic acid.

The composition may include an antioxidant and a chelating agent that inhibit the degradation of the compound. Antioxidants for some compounds are BHT, BHA, alpha-tocopherol and ascorbic acid in the exemplary range of about 0.01% to 0.3%, or BHT in the range of 0.03% to 0.10% by weight by total weight of the composition. The chelating agent may be present in an amount of from 0.010% to 0.5% by weight by total weight of the composition. Exemplary chelating agents include edetate salts (e.g. disodium edetate) and citric acid in the weight range of about 0.01% to 0.20%, or in the range of 0.02% to 0.10% by weight by total weight of the composition. The chelating agent is useful for chelating metal ions in the composition that may be detrimental to the shelf life of the formulation. While BHT and disodium edetate are exemplary antioxidant and chelating agent, respectively, for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as Arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethyl cellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, acacia, and ionic or non-ionic surfactants. Known preservatives include, but are not limited to, methyl, ethyl, or n-propyl para-hydroxybenzoates, ascorbic acid, and sorbic acid. Known sweetening agents include, for example, glycerol, propylene glycol, sorbitol, sucrose, and saccharin.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the instant specification may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as Arachis, olive, sesame, or coconut oil, fractionated vegetable oils, and mineral oils such as liquid paraffin.

Powdered and granular formulations of a pharmaceutical preparation of the instant specification may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, ionic and non-ionic surfactants, and a preservative. Additional excipients, such as fillers and sweetening, flavoring, or coloring agents, may also be included in these formulations.

A pharmaceutical composition of the instant specification may also be prepared, packaged, or sold in the form of oil-in-water emulsion or a water-in-oil emulsion. The oily phase may be a vegetable oil such as olive or Arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying. Methods for mixing components include physical milling, the use of pellets in solid and suspension formulations and mixing in a transdermal patch, as known to those skilled in the art.

### Administration/Dosage/Formulations

The regimen of administration may affect what constitutes an effective amount. The therapeutic formulations may be administered to the subject either prior to or after the onset of a disease or disorder that is affected by, associated with, or would benefit from antibacterial treatment. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions described herein to a patient, preferably a mammal, more preferably a human, may be carried out using known procedures, at dosages and for periods of time effective to treat a disease or disorder that is affected by, associated with, or would benefit from antibacterial treatment in the patient. An effective amount of the therapeutic compound necessary to achieve a therapeutic effect may vary according to factors such as the state of the disease or disorder in the patient; the age, sex, and weight of the patient; and the ability of the therapeutic compound to treat a bacterial-based disease or disorder in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound described herein is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions described herein may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds described herein employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle.

The dosage unit forms of the compound(s) described herein are dictated by and directly dependent on (a) the unique characteristics of the therapeutic compound and the particular therapeutic effect to be achieved, and (b) the limitations inherent in the art of compounding/formulating such a therapeutic compound.

In certain embodiments, the compositions described herein are formulated using one or more pharmaceutically acceptable excipients or carriers. In certain embodiments, the pharmaceutical compositions described herein comprise a therapeutically effective amount of a compound described herein and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions described herein are administered to the patient in dosages that range from one to five times per day or more. In other embodiments, the compositions described herein are administered to the patient in range of dosages that include, but are not limited to, once every day, every two, days, every three days to once a week, and once every two weeks. It is readily apparent to one skilled in the art that the frequency of administration of the various combination compositions described herein varies from individual to individual depending on many factors including, but not limited to, age, disease or disorder to be treated, gender, overall health, and other factors. Thus, administration of the compounds and compositions described herein should not be construed to be limited to any particular dosage regime and the precise dosage and composition to be administered to any patient is determined by the attending physician taking all other factors about the patient into account.

The compound(s) described herein for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In various embodiments, the dose of a compound described herein is from about 1 mg and about 2,500 mg. In various embodiments, a dose of a compound described herein used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in various embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, a composition as described herein is a packaged pharmaceutical composition comprising a container holding a therapeutically effective amount of a compound described herein, alone or in combination with a second pharmaceutical agent; and instructions for using the compound to treat, prevent, or reduce one or more symptoms in a patient of a disease or disorder that is affected by, associated with, or would benefit from antibacterial treatment.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any one of the compositions described herein include oral, nasal, rectal, intravaginal, parenteral, buccal, sublingual or topical. The compounds for use in the compositions described herein can be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions described herein are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compound(s) described herein can be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Compositions as described herein can be prepared, packaged, or sold in a formulation suitable for oral or buccal administration. A tablet that includes a compound as described herein can, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, dispersing agents, surface-active agents, disintegrating agents, binding agents, and lubricating agents.

Suitable dispersing agents include, but are not limited to, potato starch, sodium starch glycollate, poloxamer 407, or poloxamer 188. One or more dispersing agents can each be individually present in the composition in an amount of about 0.01% w/w to about 90% w/w relative to weight of the dosage form. One or more dispersing agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Surface-active agents (surfactants) include cationic, anionic, or non-ionic surfactants, or combinations thereof. Suitable surfactants include, but are not limited to, behentrimonium chloride, benzalkonium chloride, benzethonium chloride, benzododecinium bromide, carbethopendecinium bromide, cetalkonium chloride, cetrimonium bromide, cetrimonium chloride, cetylpyridine chloride, didecyldimethylammonium chloride, dimethyldioctadecylammonium bromide, dimethyldioctadecylammonium chloride, domiphen bromide, lauryl methyl gluceth-10 hydroxypropyl dimonium chloride, tetramethylammonium hydroxide, thonzonium bromide, stearalkonium chloride, octenidine dihydrochloride, olaflur, N-oleyl-1,3-propanediamine, 2-acrylamido-2-methylpropane sulfonic acid, alkylbenzene sulfonates, ammonium lauryl sulfate, ammonium perfluorononanoate, docusate, disodium cocoamphodiacetate, magnesium laureth sulfate, perfluorobutanesulfonic acid, perfluorononanoic acid, perfluorooctanesulfonic acid, perfluorooctanoic acid, potassium lauryl sulfate, sodium alkyl sulfate, sodium dodecyl sulfate, sodium laurate, sodium laureth sulfate, sodium lauroyl sarcosinate, sodium myreth sulfate, sodium nonanoyloxybenzenesulfonate, sodium pareth sulfate, sodium stearate, sodium sulfosuccinate esters, cetomacrogol 1000, cetostearyl alcohol, cetyl alcohol, cocamide diethanolamine, cocamide monoethanolamine, decyl glucoside, decyl polyglucose, glycerol monostearate, octylphenoxypolyethoxyethanol CA-630, isoceteth-20, lauryl glucoside, octylphenoxypolyethoxyethanol P-40, Nonoxynol-9, Nonoxynols, nonyl phenoxypolyethoxylethanol (NP-40), octaethylene glycol monododecyl ether, N-octyl beta-D-thioglucopyranoside, octyl glucoside, oleyl alcohol, PEG-10 sunflower glycerides, pentaethylene glycol monododecyl ether, polidocanol, poloxamer, poloxamer 407, polyethoxylated tallow amine, polyglycerol polyricinoleate, polysorbate, polysorbate 20, polysorbate 80, sorbitan, sorbitan monolaurate, sorbitan monostearate, sorbitan tristearate, stearyl alcohol, surfactin, Triton X-100, and Tween 80. One or more surfactants can each be individually present in the composition in an amount of about 0.01% w/w to about 90% w/w relative to weight of the dosage form. One or more surfactants can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable diluents include, but are not limited to, calcium carbonate, magnesium carbonate, magnesium oxide, sodium carbonate, lactose, microcrystalline cellulose, calcium phosphate, calcium hydrogen phosphate, and sodium phosphate, Cellactose® 80 (75% α-lactose monohydrate and 25% cellulose powder), mannitol, pre-gelatinized starch, starch, sucrose, sodium chloride, talc, anhydrous lactose, and granulated lactose. One or more diluents can each be individually present in the composition in an amount of about 0.01% w/w to about 90% w/w relative to weight of the dosage form. One or more diluents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable granulating and disintegrating agents include, but are not limited to, sucrose, copovidone, corn starch, microcrystalline cellulose, methyl cellulose, sodium starch glycollate, pregelatinized starch, povidone, sodium carboxy methyl cellulose, sodium alginate, citric acid, croscarmellose sodium, cellulose, carboxymethylcellulose calcium, colloidal silicone dioxide, crosspovidone and alginic acid. One or more granulating or disintegrating agents can each be individually present in the composition in an amount of about 0.01% w/w to about 90% w/w relative to weight of the dosage form. One or more granulating or disintegrating agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%4, 45%, 50%, 55%, 60%, 65%, 70%, 75%, 80%, 85%, or 90% w/w relative to weight of the dosage form.

Suitable binding agents include, but are not limited to, gelatin, acacia, pre-gelatinized maize starch, polyvinylpyrrolidone, anhydrous lactose, lactose monohydrate, hydroxypropyl methylcellulose, methylcellulose, povidone, polyacrylamides, sucrose, dextrose, maltose, gelatin, polyethylene glycol. One or more binding agents can each be individually present in the composition in an amount of about 0.01% w/w to about 90% w/w relative to weight of the dosage form. One or more binding agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55% 60%, 65%, 70%, 75%, 80%, 85%, or 90% w/w relative to weight of the dosage form.

Suitable lubricating agents include, but are not limited to, magnesium stearate, calcium stearate, hydrogenated castor oil, glyceryl monostearate, glyceryl behenate, mineral oil, polyethylene glycol, poloxamer 407, poloxamer 188, sodium laureth sulfate, sodium benzoate, stearic acid, sodium stearyl fumarate, silica, and talc. One or more lubricating agents can each be individually present in the composition in an amount of about 0.01% w/w to about 90% w/w relative to weight of the dosage form. One or more lubricating agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Tablets can be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and U.S. Pat. No. 4,265,874 to form osmotically controlled release tablets. Tablets may further comprise a sweetening agent, a flavoring agent, a coloring agent, a preservative, or some combination of these in order to provide for pharmaceutically elegant and palatable preparation.

Tablets can also be enterically coated such that the coating begins to dissolve at a certain pH, such as at about pH 5.0 to about pH 7.5, thereby releasing a compound as described herein. The coating can contain, for example, EUDRAGIT® L, S, FS, and/or E polymers with acidic or alkaline groups to allow release of a compound as described herein in a particular location, including in any desired section(s) of the intestine. The coating can also contain, for example, EUDRAGIT® RL and/or RS polymers with cationic or neutral groups to allow for time controlled release of a compound as described herein by pH-independent swelling.

### Parenteral Administration

For parenteral administration, the compounds as described herein may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1, 3-butanediol. Among the acceptable vehicles and solvents that may be employed are water, Ringer's solution and isotonic sodium chloride solution. Sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as Ph. Helv or similar alcohol.

### Additional Administration Forms

Additional dosage forms suitable for use with the compound(s) and compositions described herein include dosage forms as described in U.S. Pat. Nos. 6,340,475; 6,488,962; 6,451,808; 5,972,389; 5,582,837; and 5,007,790. Additional dosage forms suitable for use with the compound(s) and compositions described herein also include dosage forms as described in U.S. Patent Applications Nos. 20030147952; 20030104062; 20030104053; 20030044466; 20030039688; and 20020051820. Additional dosage forms suitable for use with the compound(s) and compositions described herein also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations described herein can be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use with the method(s) described herein may be administered in the form of microparticles, for example, by injection or in the form of wafers or discs by implantation.

In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions described herein. Thus, single unit dosage forms suitable for oral administration, such as tablets, capsules, gelcaps, and caplets, that are adapted for controlled-release are encompassed by the compositions and dosage forms described herein.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds. The term “controlled-release component” is defined herein as a compound or compounds, including, but not limited to, polymers, polymer matrices, gels, permeable membranes, liposomes, or microspheres or a combination thereof that facilitates the controlled-release of the active ingredient. In one embodiment, the compound(s) described herein are administered to a patient, alone or in combination with another pharmaceutical agent, using a sustained release formulation. In one embodiment, the compound(s) described herein are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound described herein depends on the age, sex and weight of the patient, the current medical condition of the patient and the progression of a disease or disorder that is affected by, associated with, or would benefit from antibacterial treatment in the patient being treated. The skilled artisan is able to determine appropriate dosages depending on these and other factors.

A suitable dose of a compound described herein can be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the compound(s) described herein is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced to a level at which the improved disease is retained. In certain embodiments, patients require intermittent treatment on a long-term basis upon any recurrence of symptoms and/or infection.

The compounds described herein can be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

### EXAMPLE

The instant specification further describes in detail by reference to the following experimental examples. These examples are provided for purposes of illustration only, and are not intended to be limiting unless so specified. Thus, the instant specification should in no way be construed as being limited to the following examples, but rather, should be construed to encompass any and all variations which become evident as a result of the teaching provided herein.

### Example 1

Medical biofilms can be defined as an infectious organized community of bacteria that are living as a multicellular entity. Bacteria have evolved multiple strategies to ensure their survival in these communities. One of these strategies is called the “stringent response” and is activated almost universally among most bacterial species in response to various nutrient limitations. The keystone enzyme in triggering the bacterial stringent response which produces a state of relative metabolic quiescence is RelA. RelA is a highly conserved pyrophosphotransferase that is encoded by the relA gene. This enzyme is responsible for sensing amino acid starvation at the ribosome and is self-regulating within a bacterial biofilm. RelA catalyzes the reaction between GTP/GDP and ATP to form the “alarmones” pppGpp (guanosine pentaphosphate) and ppGpp (guanosine tetraphosphate). These molecules trigger a transcriptional change within the cell causing an upregulation in oxidative damage combating enzymes as well as a reduction in replication machinery. Cells which have gone through these metamorphic changes are called persister cells. Persister cells, while not genetically resistant to antibiotics, can withstand up to 1000× the antibiotic concentrations of their planktonic counterparts.

This work outlines the task of developing a RelA inhibitor which would both prophylactically stop persister cells formation and would convert already formed persister cells to their antibiotic-susceptible state allowing for the synergetic treatment of medical biofilms using a combination therapy. An in silico pipeline approach to identify hit compounds for the inhibition of RelA was developed. Using this approach, combined with biochemical, molecular biological and microbiological methods, the present study has analyzed these in silico hit compounds and identified a set of lead compounds that inhibit RelA and reduce biofilm persistence when the biofilm is simultaneously treated with antibiotics and the RelA inhibitors. The present study developed and synthesized several analogs of these lead compounds and have tested them in SAR studies to show how these compounds interact in the inhibition of the RelA enzyme. Through this approach, a better understanding of the stringent response and the ability to combat bacteria in medical biofilm infections were both achieved.

In the early 2000s, the U.S. National Institutes of Health announced that antibiotic resistant bacterial biofilms are detrimentally relevant in over 80% of infections in the human body. It is estimated that biofilm infections cost the U.S. health-care system on the order of 5 billion dollars per year. A medical biofilm can be defined as an infectious organized community of bacteria living as a multicellular entity. With this enormous role in clinical infections, it is imperative to find a way to inhibit biofilm resistance and add this method to the arsenal of conventional antibiotics. Currently, the standard of care for the treatment of bacterial biofilms includes debarment and mechanical removal of biofilm infections.

To combat starvation and environmental stresses, bacteria have evolved a strategy to ensure their survival. This same strategy, called the “stringent response”, is responsible for the bacterial biofilm's ability to evade the host immune system and antimicrobial chemotherapeutic treatment. This response is activated almost universally among bacterial species. Individual bacterial cells, unless they are genetically resistant, are easily eradicated by current antibiotics. However, these same bacteria when growing as biofilms can be resistant to 1000 times the antibiotic concentrations of their planktonic envirovars. Antibiotic resistance of bacterial biofilms has been shown to be metabolic, in that most bacteria in a biofilm are relatively metabolically quiescent. This metabolic resistance can be partially overcome by providing fermentable substrates or alternate e⁻ acceptors. This metabolic resistance of biofilms to antibiotics is an active, not passive, process and relies on the triggering of the bacterial stringent response from the protein RelA.

The RelA protein produces the alarmones guanosine pentaphosphate [(p)ppGpp] and guanosine tetraphosphate (ppGpp), referred to as the “magic spot”. The magic spot induces a major change in cellular metabolism whereby the bacteria are converted into persister cells (like human stem cells). Persister cells, like stem cells, upregulate many enzymes involved in reducing oxidative damage. Because essentially all antibiotics kill by vastly increasing oxidative stress through interruption of macromolecular syntheses, the biofilm persister cells become recalcitrant to antibiotics. These cells have already upregulated the enzymatic machinery to protect from the effects of antibiotics. Thus, biofilms can form independent of the triggering of the stringent response, and thus it is possible to construct bacteria that have had their relA gene removed. These bacteria form biofilms but cannot enact the stringent response. If these relA knockout biofilms were treated with antibiotics, the bacteria die much easier. Thus, RelA becomes the target of choice for the design of anti-biofilm drugs.

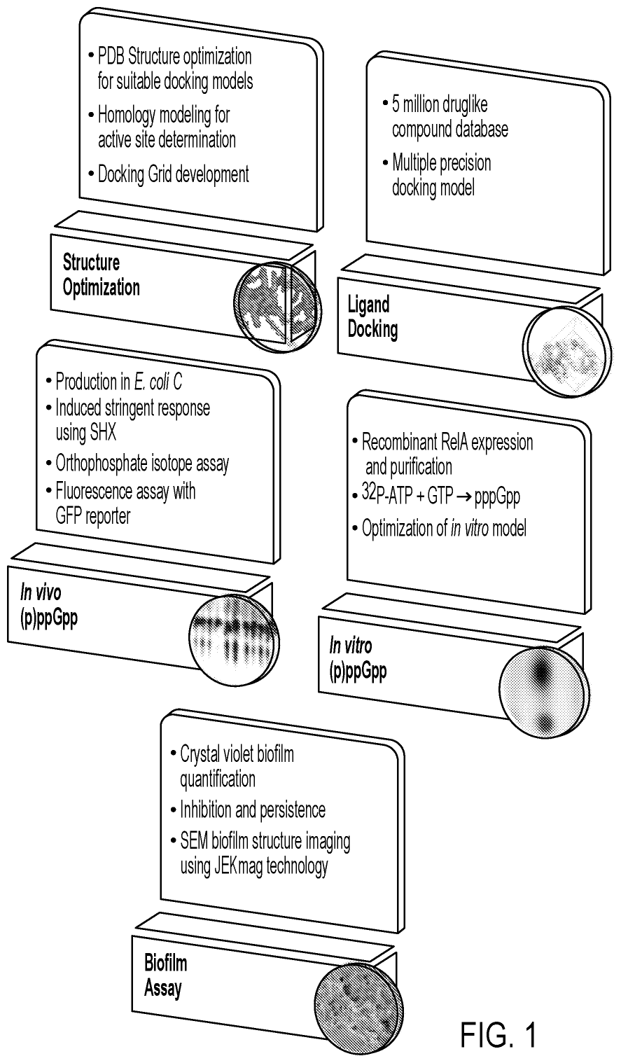

In one aspect, the present study is directed to the design and experimental characterization of small-molecule inhibitors for the magic spot alarmone-producing enzyme RelA. The use of a carefully constructed pipeline outlining the preclinical drug design process was developed (FIG. 1). This process has led to hit compounds shown to repotentiate antibiotics for the killing of bacterial biofilms.

### Example 1-1: Biofilms

### Origins of Biofilms

The term “biofilm” was first coined by Bill Costerton in 1987 in the paper “Bacterial Biofilms in Nature and Disease.” The study of biofilms can be traced back to the “father of microbiology” Antonie van Leeuwenhoek circa 1683 and the use of his simple microscopic techniques on teeth plaque to observe what he called “animalcules”. These animalcules he observed were bacteria that had formed microbial communities. It has been speculated that biofilms were essential to the success of life in the early stages of evolution from the first replicating cells. These early cells needed to combat the hostile environment of the early Earth. The Earth at this time had very specific areas that could support their development. If bacteria were to survive, they could not separate from these nutrient sources. Thus, communities of bacteria that are attached to a surface would be beneficial to species survival.

From this early example of biofilms trying to survive on the primordial Earth to present-day biofilms trying to evade host immune systems and antimicrobial chemotherapies, biofilms are about species survival. The notion that the collective is greater than the sum of its parts is essential to the biofilm paradigm. There are many formal definitions of biofilms; however, the common theme among all the definitions is the organized community of unicellular organisms living as a multicellular entity.

### Development and Formation of Biofilms

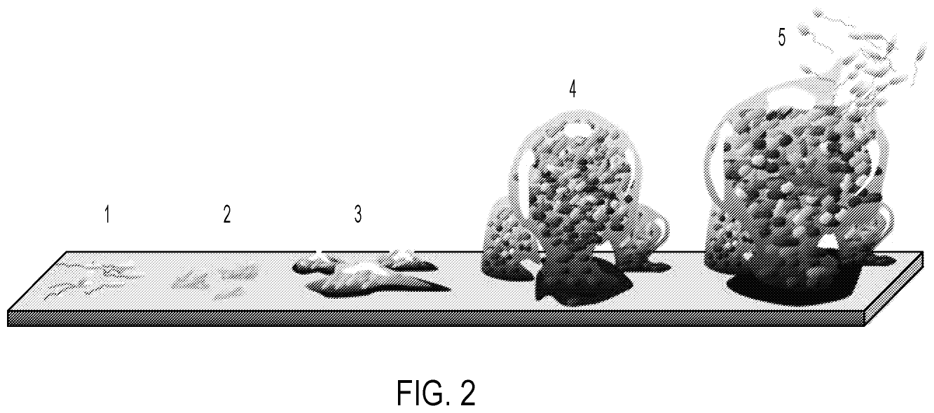

There are 5 main stages (FIG. 2) to the development and formation of biofilms in both medical and environmental settings. These stages outline the biofilm lifecycle.

Initially the planktonic bacteria are free floating in the bulk, and when they come in contact with a surface, the attachment is considered to be reversible at this stage and there is generally just a physical attachment to the surface. The factors that outline the initial attachment depend on the medium environment (nutrient concentration, pH, temperature, oxygen concentration) and materials surface, as well as the bacterial species and strain. At this stage, the bacteria can leave the surface easily and it is only once there is formation of an extracellular polymeric substance (EPS) that the irreversible attachment of the biofilm structure is observed.

The initial formation of the EPS encapsulates the cells as they aggregate together. At this stage, the biofilm is a cell layer thick. The irreversible attachment stage is succeeded by a set of growth stages. Stage 3, also known as maturation I, is when the 3D structure of the biofilm becomes pronounced. During this stage, the biofilm may also “recruit” other species of bacteria into the matrix.

Stage 4, or maturation II, is one of the most eventful stages of the biofilm lifecycle. As the biofilm grows larger, there becomes heterogeneity within the biofilm. There are biofilm regions that experience different conditions, including nutrient and oxygen limitations, pH gradients, and even cellular diversity in mixed-species biofilms. In this stage, one of the quintessential shifts in metabolism occurs. As the limitation in nutrients occurs, the stringent response is activated in groups of cells. These cells shift to what are known as persister cells. As the name indicates, these cells have undergone a gene regulation change that allows them to persist.

The final stage of the biofilm cycle is the dispersion of the biofilm. This is sometimes referred to as planktonic showers. During this stage, cells detach or are sluffed off from the biofilm structure. In medical biofilms, these cells can cause systemic infections, which can be reoccurring. These now planktonic cells can start the lifecycle over, causing the formation of daughter biofilms.

### Matrix and Extracellular Polymeric Substances

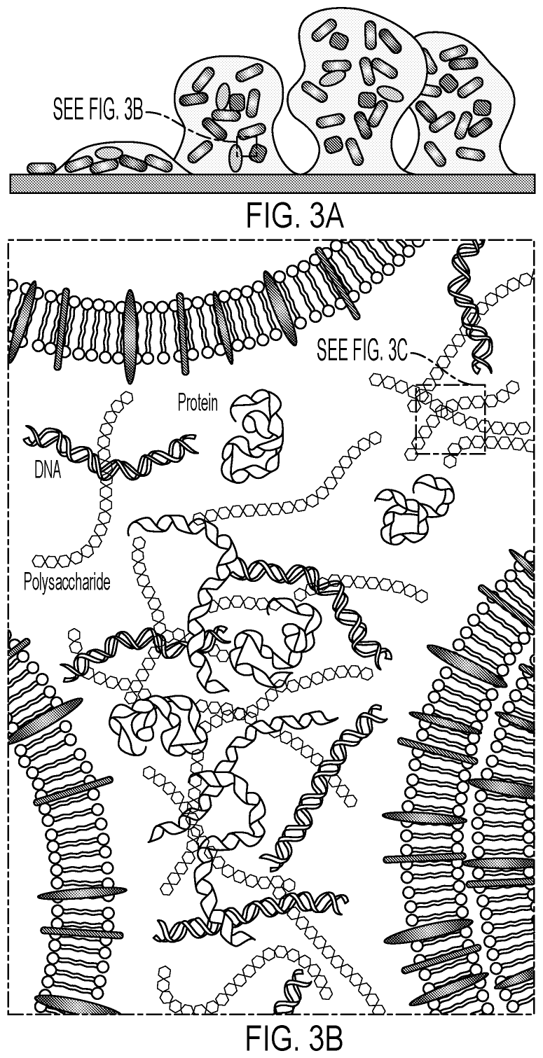

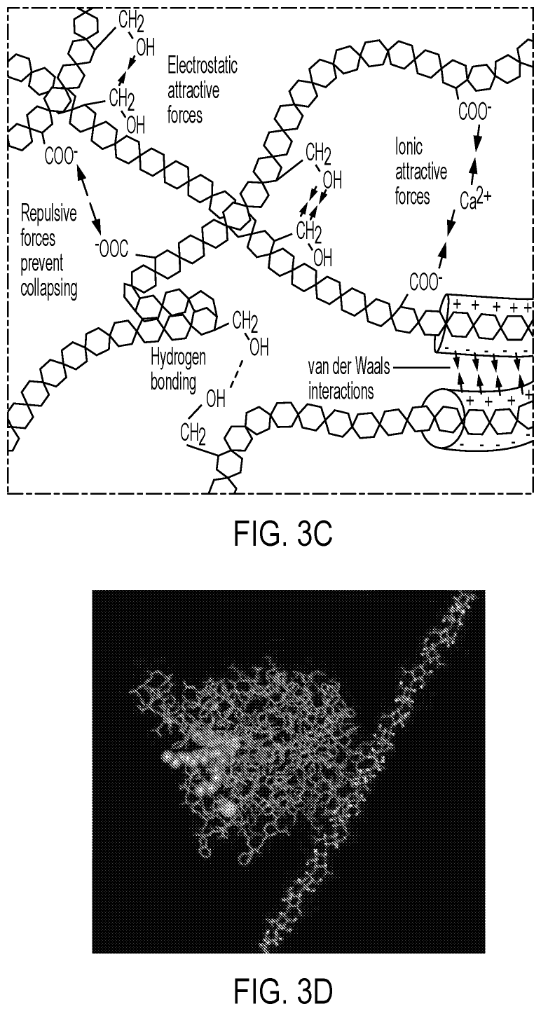

The EPS is the matrix that embeds cells in a biofilm. This complex “slime” is composed of many biopolymers (polysaccharides, proteins, and extracellular DNA [eDNA]) and lipids (FIGS. 3A-3D). These polymers form a hydrogel and make up anywhere between 50 and 90% of the biofilm's mass. The matrix has many notable functions but becomes essential in the protection of bacteria in many environments.

The largest portion of the EPS is made of polysaccharides and can be neutral or anionic in charge. To date the largest portion of studied biofilms are composed of heteropolysaccharides with the incorporation of some negatively charged sugars into the structure. The anionic nature of some of the EPS results in the crosslinking and toughness of the biofilm structure. FIG. 3C shows the intermolecular forces that are represented in biofilm. Hydrogen bonding, van der Waals, and ionic crosslinking are the major components that determine the structural properties of the biofilm.

Extracellular biofilm proteins, by weight, can even exceed the polysaccharide content of a biofilm. There are structural proteins, as well as active enzymes, within a biofilm matrix. The true number and type of enzymes found within a biofilm are unknown for every biofilm-forming species; however, a few main classes have been identified. The major classes include protein-degrading enzymes, (poly)oligosaccharide-degrading enzymes, lipid-degrading enzymes, and oxidoreductases. The main role of these enzymes is extracellular digestion, which plays a major role in acquiring carbon sources for cells from the breakdown of matrix components. These extracellular enzymes also play a role in the “planktonic shedding” of late-stage biofilms.

Structural proteins also play an important role in the biofilm structure and the attachment of cells to the biofilm structure. Many bacteria are able to form linkages with the biofilm matrix using surface-decorated proteins called lectins. Another main protein found in bacteria such as Streptococcus aureus is biofilm-associated surface protein. These surface proteins are high contributors to biofilm formation in several bacterial species. The last set of structural proteins found in a biofilm matrix is proteinaceous appendages, including such structures as pili and fimbriae. These structures can act as crosslinking proteins within the biofilm and as matrix stabilizers.

The presence of extracellular DNA as a structural matrix material was described. The finding was that Pseudomonas aeruginosa biofilms treated with deoxyribonuclease showed biofilm degradation. It is now known that DNA, specifically eDNA, plays a vital role in biofilm matrix stabilization, transferring genetic material (horizontal gene transfer) and helping biofilm's conditioning immune responses.

### Biofilms in Infectious Diseases

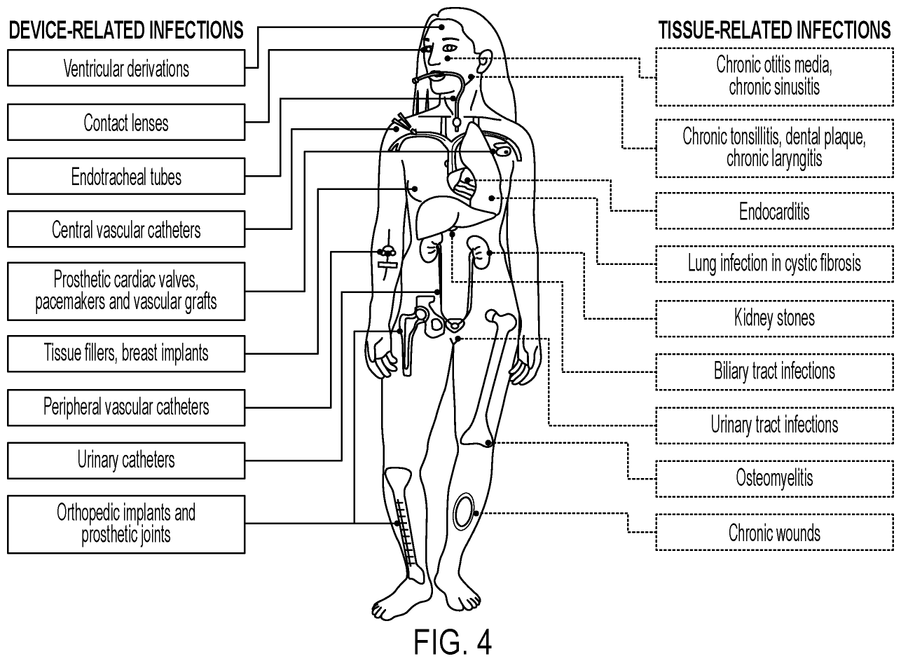

Biofilms, as part of an infectious disease, have become a major burden on the health-care industry, causing more than 5 billion dollars of cost in the United States alone. There are a number of known chronic infections caused by biofilms (FIG. 4), the most common being infected arthroplasties, bony non-unions, chronic wounds, cystic fibrosis pneumonias, chronic otitis media with effusion, chronic sinusitis, chronic pelvic pain syndromes, and lower back pain associated with biofilm infections in the nucleus polposa of the vertebral disks.

Current protocols for the disruption and degradation of biofilms as treatment for antibiotic-resistant infections include iron-chelating compounds and preventative treatments, all of which suffer from low efficacy. The debridement of biofilms or removal of arthroplasty implants is currently the main treatment strategy. In certain embodiments, an anti-biofilm therapeutic is paradigm shifting and allows for the noninvasive treatment of biofilm infections.

### Example 1-2: Stringent Response

The stringent response in bacteria is a global gene regulation change that puts the cells in survival mode. In bacteria, this process is triggered by the RelA/SpoT Homology (RSH) enzymes, which catalyze the reactions ATP+GTP→AMP+pppGpp and ATP+GDP→AMP+ppGpp, where ATP=adenosine triphosphate and GTP=guanosine triphosphate. Both (p)ppGpp and ppGpp serve as alarmones that initiate global changes in gene expression, which evolved to convert the cell's metabolism from growth and division to survival.

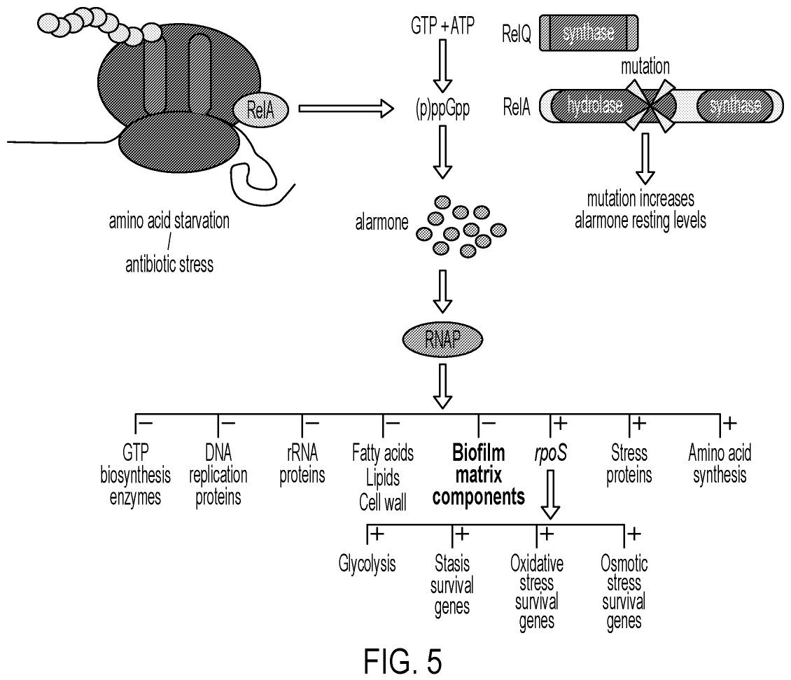

As originally characterized, the stringent response was a mechanism to sense a lack of charged tRNAs at the ribosome, which would indicate an insufficient pool of amino acids. Subsequently, it was learned that other types of precursor limitations (iron, fatty acids) could trigger the stringent response, as well as stress conditions such as heat shock and oxidative stress. In Escherichia coli, this upregulation of stress response is highly dependent on the RNA polymerase sigma factor S (FIG. 5).

The stringent response results in a metabolism shift, turning the bacteria into persisters, the equivalent of mammalian stem cells. Stem cells and persisters owe their extraordinarily long-term survival to the upregulation of proteins associated with combating oxidative stress. Since all antibiotic killing is ultimately through the production of reactive oxygen species associated with the disruption of various macromolecular processes, this upregulation of protective enzymes is what gives biofilms their antibiotic resistance phenotype.

### Example 1-3: RelA and the Magic Spot Alarmones

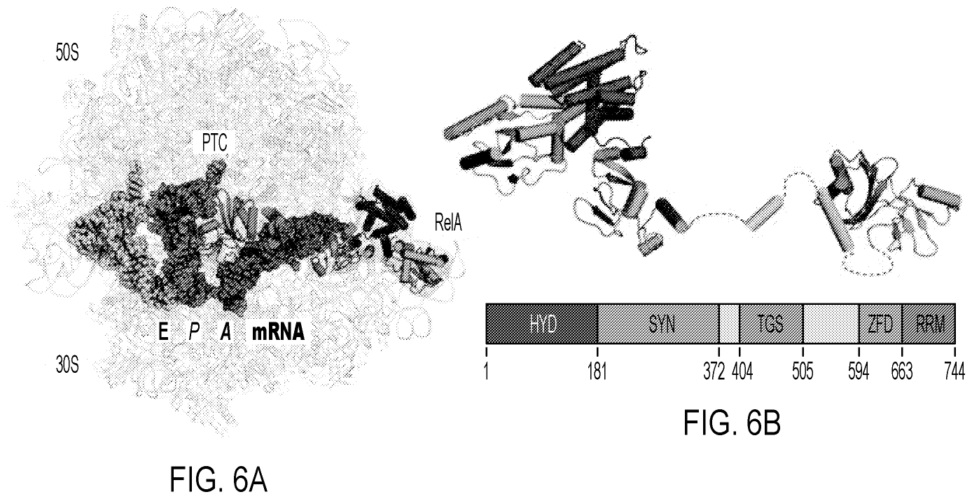

The RelA enzyme is a 84-kD pyrophosphokinase protein (FIGS. 6A-6B6). RelA is closely associated with the ribosome and detects amino acid starvation. This starvation triggers the bacterial stringent response that leads to the phenotypic changes underlying the extreme biofilm recalcitrance to antibiotics. Thus, this RelA-mediated ancient bacterial stress response produces an active metabolic state that results in the inability to treat chronic infections caused by biofilms.

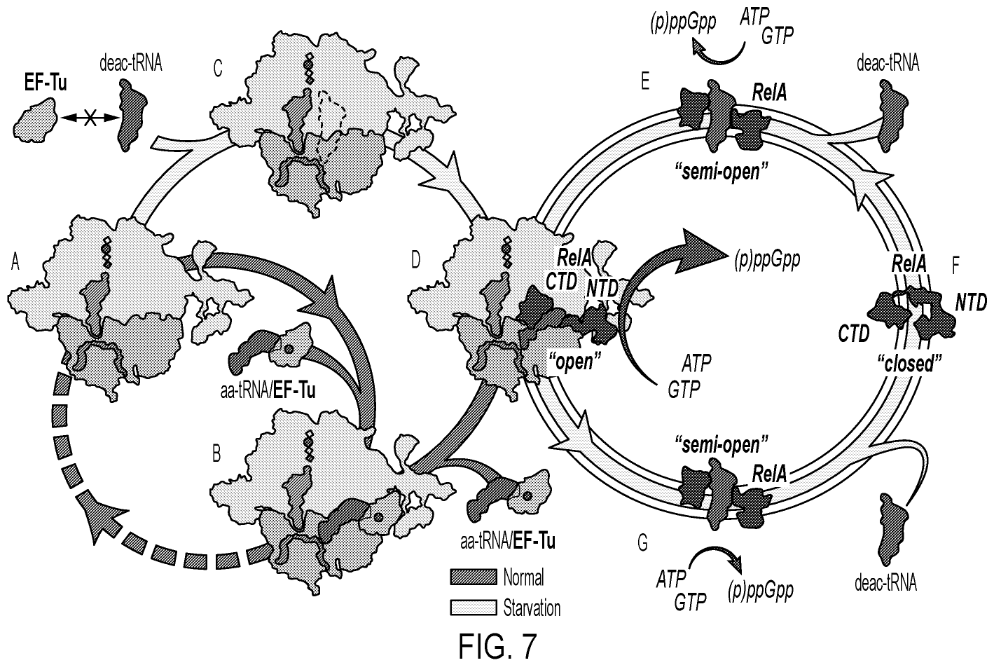

For more than half a century, the magic spot alarmones, guanine tetraphosphate and guanine pentaphosphate, collectively known as (p)ppGpp, have been known to play an integral role in cell signaling for the stringent response. In E. coli, the production of (p)ppGpp is carried out by the enzyme RelA.65 RelA displays a well-choreographed dance with ribosomes to detect amino acid starvation by means of stalled deacetylated tRNAs (FIG. 7).

Upon detection of this stalled, uncharged tRNA, RelA subsequently binds to the ribosomal complex and structurally changes to its active synthase conformation. While in this “open” conformation, RelA continually produces (p)ppGpp. During this time, the intracellular concentrations of (p)ppGpp increase dramatically. The increased concentration of (p)ppGpp modulates multiple downstream cellular signaling pathways, including interacting with the rRNA polymerase's promoter binding region, thereby interfering with the cell's ability to produce additional ribosomes.

The active domain of RelA encompasses amino acid residues 181-372. While characterization of the catalytic mechanism in RelA has not been fully determined using structural analysis, there has been work on a simpler RelA/RSH RelP. RelP and RelA have highly conserved interacting residues within the active domain based on both genomic and structural homology studies.

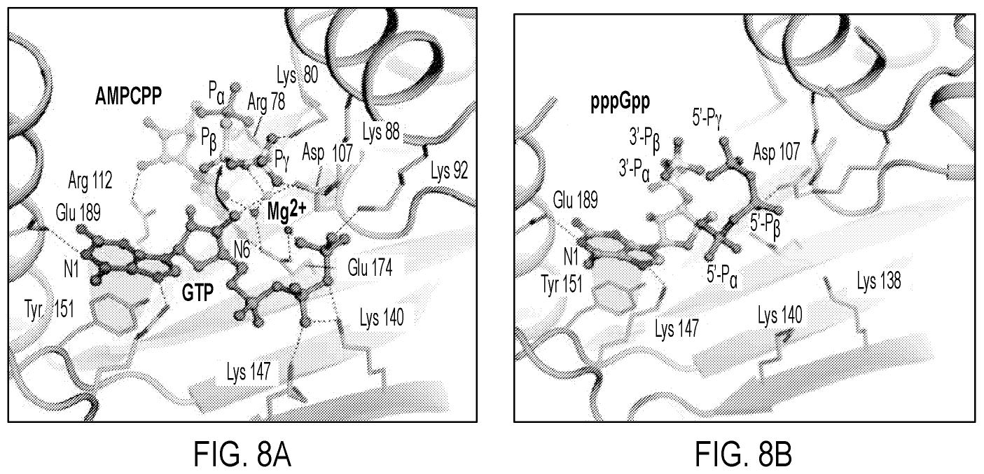

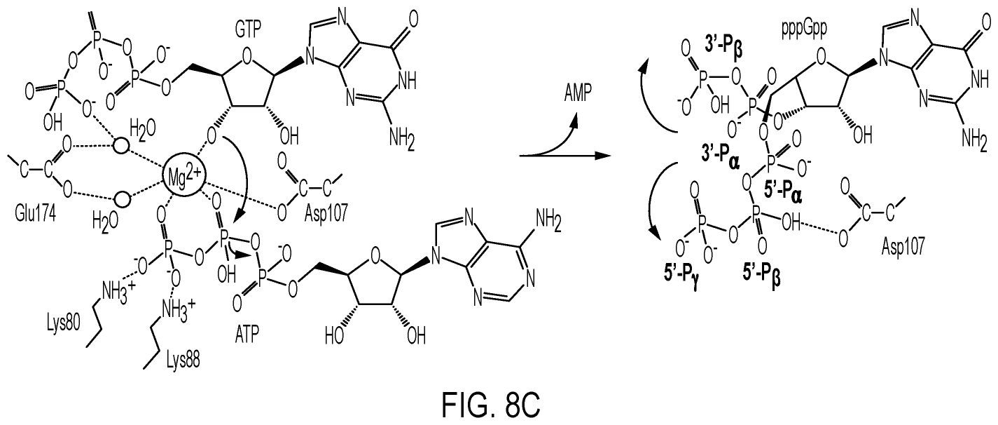

The catalytic mechanism of RSH enzymes is hypothesized to be very similar among all RSH enzymes and is outlined in FIGS. 8A-8C. RSH binds GTP, which stabilizes the Mg ion to several negatively charged residues in the active site and to the γ-phosphate of GTP. ATP then coordinates via its β- and γ-phosphates. Magnesium facilitates an electron transfer from the 5′hydroxyl to the β-phosphate phosphorus, relieving the pyrophosphate from ATP and transferring it to the GTP in a catalyzed SN2-type reaction.

### Example 1-4: Structure-Based Drug Design: Molecular Docking

In silico structure-based drug design is the field of study of producing bioactive compound by looking at the atom arrangements of a target protein, a known inhibitor, or a set of ligands. Using this model, a large group of in silico compounds can be screened using various methods. These compounds can then be ranked using a metric that gives information on the binding of the compound to the target biomolecule. One non-limiting advantage of this paradigm is the ability to screen millions of compounds at low cost and relatively high speeds, compared to a high-throughput screening process.

With the advent of protein structure analyses such as nuclear magnetic resonance (NMR), x-ray crystallography, and more recently cryo-electron microscopy, a protein structure can be elucidated quickly and at high resolutions. The ability to “see” a protein opened the possibilities of designing a compound that complements a region of the protein. Co-crystallizing a ligand bound to a receptor and generating a structure allowed for accurate determination of ligand-receptor interactions.

### Structure-Based Drug Design: Molecular Docking

The goal of today's molecular modeling with regard to molecular docking is to determine a ligand that fits inside a receptor and rank the ligands based on some biologically relevant scoring function. To perform a successful docking simulation, a target and a set of ligands need to be optimized. As of July 2020, the Protein Data Bank (PDB) contains 166,301 structures. This vast library of targets enables easy access to relevant proteins and enzymes to utilize in docking models. Libraries of ligands can be millions of compounds large and span the entirety of chemical space.

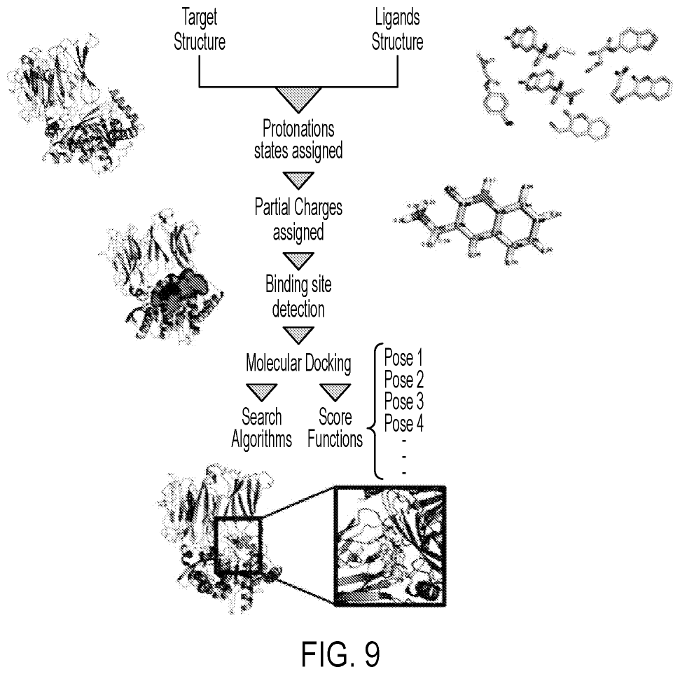

The general workflow for a molecular docking pipeline starts with a target structure and is outlined in FIG. 9. A target structure is a protein structure that has some biologically significant role that, when acted on by a ligand, changes its function. In many cases, the structures found on the PDB database have information about an active site, by means of either a co-crystallized ligand or a homology study of similar proteins. A protein needs to be prepared before a docking calculation can be performed. This preparation rectifies any issues from the structure that might be present from obtaining the structure, including missing side chains, clashing residues, rotamer corrections, protonation states, and removal of ligands. The docking model is only as good as the target and ligands that go into the model; therefore, these steps are critical for an accurate model.

The ligands that are docked in the model need to be prepared in similar ways to those of the protein. Ligand databases are stored as large text files that give atom coordinates and general information about that molecule. These databases need to be constructed in such a way that they are readable to the docking software and contain all the information needed for a docking model to be performed. Ligands need to be geometry optimized and each ligand has several poses. The vast amount of previous calculations on druglike molecules using molecular mechanics has contributed to the Optimized Potentials for Liquid Simulations (OPLS) force fields. These force fields have been developed specifically to predict low-energy geometries and potential docking poses. One such force field is OPLSe, developed with ligand docking at its core. Because electrostatic interactions are essential in protein-ligand systems, proper protonation is also essential to ligand database development. Ligand databases may have several final structures from one input compound. This expanded ligand set allows for the most biologically relevant molecules to be represented in the docking model.

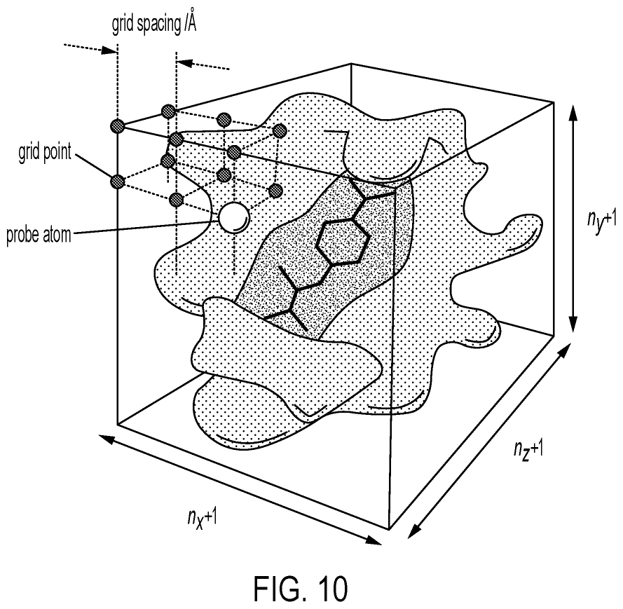

To simplify the calculations in molecular docking experiments, the target protein is not treated as an entire system. Instead, the target site is determined using known homology, co-crystallized ligands bound in the site of interests, or general knowledge of the activity of the protein of interest. The site is isolated, and a grid is created. A docking grid is a 3D map of the site of interest (FIG. 10). It stores information about the site using regularly spaced grid points. A grid is developed by creating a node at regularly spaced intervals. At this node, a set of probe atoms is placed at each node point. These atoms represent all atoms in the set of ligands to be docked. Depending on the database of ligands docked, this probe atom can be positively charged, negatively charged, or remain neutral. The energy at each of these nodes is recorded and placed in a table that is referenced when the docking is performed. Grid development takes the longest time and is the most computationally demanding portion of the docking process.

Once a set of ligands and the docking grid are prepared and optimized, based on the biologically relevant system that is probed, it is placed into a docking model. There are several approaches to a docking calculation, including GOLD and AutoDock. Employed docking models in this thesis are an increasing precision pipeline using Schrödinger Glide. Schrödinger Glide has three precision docking algorithms: high-throughput virtual screening (HTVS), standard precision (SP), and extra precision (XP). Each of these algorithms has its advantages and disadvantages. Glide HTVS can run quickly and give initial hits based on geometry of binding and simple force field calculations. This algorithm runs about 2 seconds per ligand. The SP algorithm runs slightly slower at a speed of 10 seconds per ligand but runs an extensive sampling of poses and more accurately gives docking scores.

The final algorithm to be run, XP, is the most rigorous of these docking models and calculates a combination of energies. Equations 1.1-1.3 outline what comprises a Glide XP Score. These equations are also used in the Schrödinger docking methods in Examples 2 and 3 below. The Glide score is the final ranking system for ligands bound to target receptor. This score is composed of the several energies, columbic interactions, van der Waals forces, energy of binding, and a penalty score. The energy of binding (Equation 1.2) accounts for several interactions in the binding event and is what sets Glide XP apart from other binding programs. Ehyd_enclosure is a calculation of the hydrophobic interactions. Ehb_nm_motif is an energy assigned to neutral-neutral hydrogen bond motifs and is a consideration of special-case hydrogen bonds formed in the receptor. Ehb_cc_motifs is like the previous term except it accounts for charged-charged hydrogen bonds. EPI accounts for pi-cationic and pi-stacking interactions. Ehb_pair and Ephobic_pair are common terms and are hydrogen bond energy and lipophilic pair energy, respectively. The last term in the Glide score is the Epenalty, which is the energy associated with an unfavorable interaction and consists of two terms, Edesolv and Eligand_strain. The Edesolv is the energy required for the receptor waters to be displaced by the binding ligand. The last term is the Elignad_strain, the energy accounting for the strain placed on the ligand from deforming from its favorable geometry. All these energies account for the binding score and ranking system in Glide docking. All in all, there are around 80 different parameters that go into a Glide XP scoring function, these being based on empirical data and large training sets.

$$XP Glide Score = {E}_{coul} + {E}_{vdW} + {E}_{bind} + {E}_{penalty} \qquad ( 1.1 )$$

$${E}_{bind} = {E}_{hyd\_enclosure} + {E}_{hb\_nn \_motif} + {E}_{hb\_cc \_motif} + {E}_{PI} + {E}_{hb\_pair} + {E}_{phobic\_pair} \qquad ( 1.2 )$$

$${E}_{penalty} = {E}_{desolv} + {E}_{ligand\_strain} \qquad ( 1.3 )$$

The XP Glide docking model is carried out using the following steps, which have been fully described by Friesner et al. First, the model works by running a SP docking calculation. This SP docking is a function that does not incorporate the penalties associated with XP and it identifies ligands that have a reasonable fit to a receptor site. The SP docking identifies moieties of the ligand structure docked strongly to the receptor site. These strong interactions act as “anchors” for the ligand to the receptor. These receptors are most often areas of low degrees of freedom (rings and rigid portions of the ligand). The model then optimizes them one chain at a time based on the corresponding receptor grid. Any steric clashes are discounted as possible ligand confirmations. The ligand confirmations not immediately discounted are then ranked. Top scoring ligands are minimized and scored using molecular mechanics energy and empirical parameters and ranked again. Then a scoring function ranks based on the water dissolution grid-based function. The final calculation is ligand strain penalties, which contribute negatively to the binding. Finally, the scoring functions are summed with all the energies captured in Equation 1.1. Glide is currently the highest-end protein-ligand docking system. It employs the most parameters and has been tested on targeted systems that have shown it outperforms other docking algorithms.

### Major Limitations of Molecular Docking

While molecular docking is an effective approach in drug discovery it does have its limitations. Several limitations arrive from the protein structure; these can be from resolution issues, residue confirmation issues, or just lack of dynamic information. Others are inherent to docking simulations themselves; these include the molecular mechanic calculations or the weights that are placed on each of the forces tabulated to give the final docking score.

In regards to limitations from protein structure while there are many proteins that have well established structures there are many lower resolution structures available. These structures do not have accurate density data to predict bind pocket residue orientations. For the structure to be utilized in docking simulations a resolution of <3 Å is recommended. This cutoff resolution limits the number of structures available for docking studies.

The second issue that occurs when using a docking model is the confirmation of the active site when calculations are performed. A protein with many structures available can alleviate this in some respects. If there are known pre and post catalytic structural information available a more dynamic picture of the active site can be established for the docking model. Ultimately this limitation comes from the lack of dynamic simulations in docking modeling. Other computational tools such as post docking optimization and modular dynamic simulations are ways to improve the accuracy and validity of the model.

The limitation that are inherent to docking simulations are in the calculations themselves. These calculations often omit solvent entropy effects as well as overestimate terms such as H-bonding (except Glide XP). In many instances water is essential to the ligand binding event, docking software does not account of possible water target site interactions as well. Because of this the use of molecular docking to get experimentally observed values is not practical. However, docking gives a scoring function and a rank order of the possible ligand-protein interactions. With the advancement of new scoring functions these docking simulations could possibly produce ballpark experimentally produced values.

Molecular docking is an essential tool in the toolkit of medicinal chemists and chemical biologists. Without these docking studies, it would be improbable to search the full chemical space to discover novel inhibitors. These high-throughput docking studies allow millions of molecules to be tested in reasonable timeframes and at low cost compared to those of laboratory high-throughput screening. Utilizing these effective algorithms, as well as biophysical, enzymatic, and cellular assays, effective hit compounds can be discovered. These compounds can then be entered into a computational structure-activity relationship pipeline for ligand optimization. This iterative process builds upon the laboratory results, as each iteration contributes to the in silico design of more effective inhibitors.

### Example 2: Initial Hit Compounds S3-G1A & S3-G1B and Validation of Pipeline Construction

In Example 2 the initial pipeline development and the validation of the pipeline for RelA inhibitors using two initial hit compounds are described. Example 2 also outlines the initial in silico docking studies and the target site validation through binding site residue modification and enzyme homology modeling. Compounds were determined and tested in in vitro and in vivo biological assays. An in vivo (p)ppGpp reporter system is described and utilized.

### Example 2-1: Overview

Currently, there are a very limited number of inhibitors known for RelA and (p)ppGpp that have been identified, principally, through traditional drug discovery methods, such as substrate analog design and high-throughput compound screening, none of which are candidates for clinical trials for the control of biofilm infections. The first of these inhibitors were analogs to ppGpp itself, such as Relacin and its analogs. These compounds, while mildly effective, suffer from off-target effects and low binding affinities.

The next compound discovered to reduce the intracellular concentrations of ppGpp was the cationic peptide known as IDR1018. This peptide is an analog to bactenecin and it was reported to directly sequester and break down (p)ppGpp, thus lowering its intracellular concentration. It is now thought that IDR1018 does not specifically target (p)ppGpp, but simply acts as an antimicrobial agent by means of its cationic nature. Moreover, IDR1018 is a moderately sized peptide incapable of being an orally administered “druggable” compound.

Recently, a trend toward the use of in silico chemistry and molecular modeling for computer-aided drug design has gained significant momentum. Previously, this was impossible to do with the RelA/RSH (RelA SpoT-homolog) family of enzymes, as there were no adequate high-resolution molecular structures available. However, several RelA and related enzyme structures have been characterized and published: RelA (E. coli), RelP (Staphylococcus aureus), RelQ (S. aureus)15, Relseq (Streptococcus equisimilis), and Rel (Mycobacterium tuberculosis). Thus, it has become possible through alignment and homology studies to determine the active residues within the catalytic center of these enzymes and to specifically target this region to predict and understand the ligand binding events for the rational identification of inhibitors.

Structural modeling of the E. coli RelA protein was performed to identify the active center. The present study then constructed multiple single amino acids substitution mutants of RelA based on this molecular modeling to confirm the location of the enzyme active center, and to confirm the critical role that the tyrosines, Y310 and Y319, play in its enzymatic activity. Using the structural information gained from the in silico and laboratory studies, the present study then developed a computationally-based pipeline, to identify RelA inhibitors from large databases of known compounds, that provided for the screening of compounds in a relatively timely and cost-effective manner. Millions of compounds were screened in a matter of weeks and the “hit” compounds were purchased for functional studies to determine their initial efficacy in laboratory-based in vivo and in vitro assays. The compound databases used for screening were designed to only include compounds that meet the “drug-like” criteria for ligands as defined by Lipinski's rule of 5. This method has been shown to be highly effective in the discovery of drugs over the last 20 years and continues to improve in accuracy as the algorithms for ligand docking improve19. Using these in silico docking studies, two small-molecule compounds that were predicted to inhibit the RelA enzyme were identified. These compounds were then subjected to in vivo and in vitro (p)ppGpp quantification assays using the E. coli strain C and recombinant E. coli RelA enzyme, as well as in biofilm inhibition assays using the E. coli C biofilm model.

### Example 2-2: Validation of the RelA Activity Assays

Several methods to study the RelA enzymatic activity in vitro and in vivo have been published, and the methods used herein were adapted from these sources. The present study performed two kinds of RelA activity tests: a ppGpp-dependent fluorescent reporter in vivo assay and direct (p)ppGpp detection assays in vivo and in vitro. The first method was based on the ability of ppGpp to affect expression of different genes. One of these genes rpsJ, encodes the 30S ribosomal protein S10. Its promoter, PrpsJ, belongs to the r-protein family of promoters, which are strongly inhibited by ppGpp and the DksA transcriptional factors25-26. Recently, a plasmid construct carrying a yfp (yellow fluorescent protein) gene driven by the PrpsJ was published. The reporter plasmid contains the broad host range RK2 minimal replicon and is compatible with many other plasmid vectors. Comparison of the yellow fluorescent protein (YFP) activity between wild-type (WT) E. coli K12 and its relA-mutant confirmed the effect of ppGpp production on PrpsJ activity and served as validation of this method.

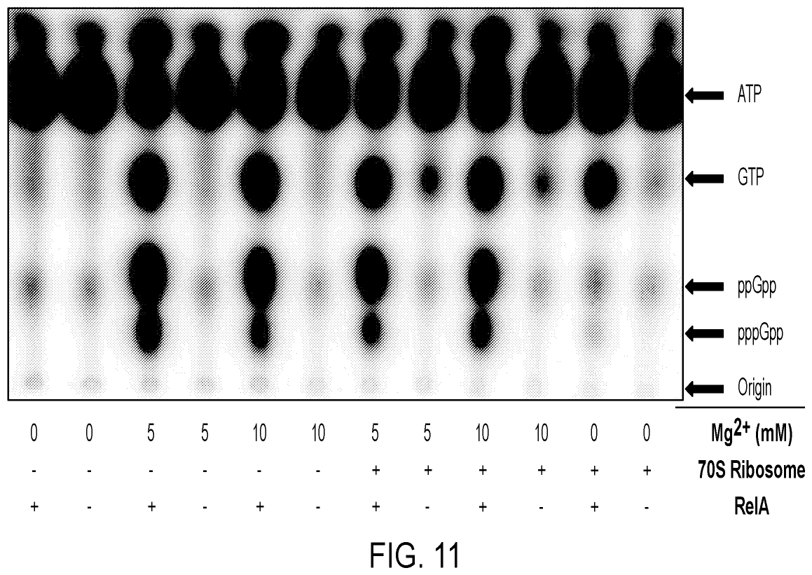

The direct (p)ppGpp detection in vivo and in vitro assays relied on different 32P radioactive nucleotides (γ-32P-ATP, α-32P-GTP) for use as substrates, and thin-layer chromatography (TLC) to separate the reaction products (stationary phase: polyethylenimine cellulose). Several methods were tested and optimized to give the best results for assessing the production of (p)ppGpp. It was found that the in vitro buffer system did not need to be phosphate free as previously indicated. It was also found that the concentration of magnesium needed to be above 5 mM for optimal synthesis of (p)ppGpp. Previous work had indicated that the 70S ribosome was needed for RelA to produce (p)ppGpp in vitro; however, the present study found this not to be the case. There was no difference observed with 5 mM MgCl₂ with and without 70S (FIG. 11); therefore, it was not used in the in vitro reactions.

In the case of the in vitro assay, it was found that using γ-32P-ATP was optimal to study the production of both ppGpp and pppGpp, while α-32P-GTP was optimal for studying only pppGpp. In the case of the in vivo studies, [32P]-orthophosphate was used as the radiation source, and the cells then incorporated the 32P into (p)ppGpp. Both methods required TLC with a stationary phase of a polyethyleneimine (PEI)-cellulose plate and a mobile phase of 1 M potassium phosphate monobasic.

### Example 2-3: Homology Studies

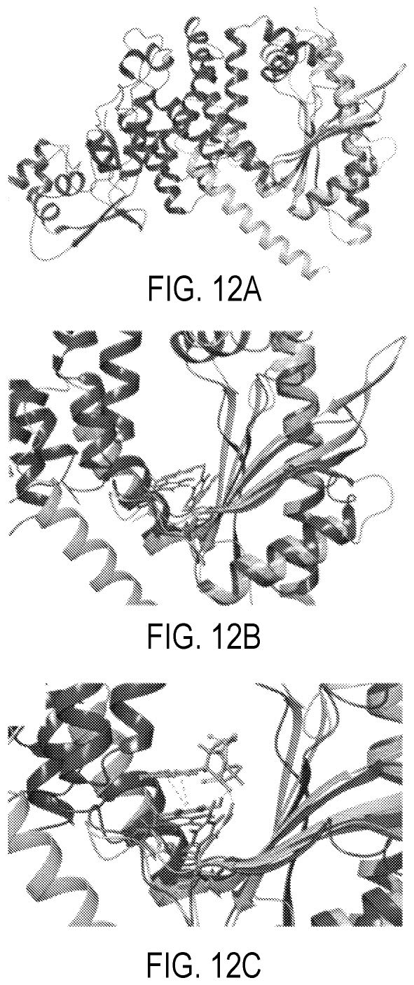

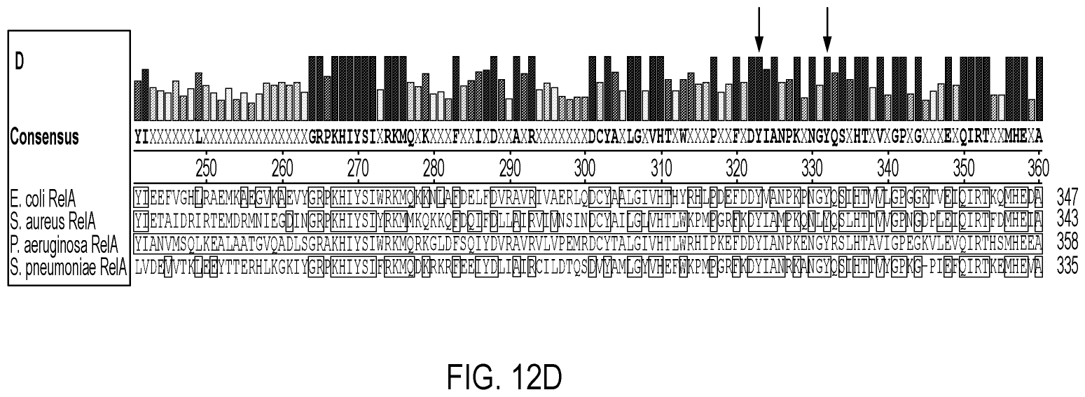

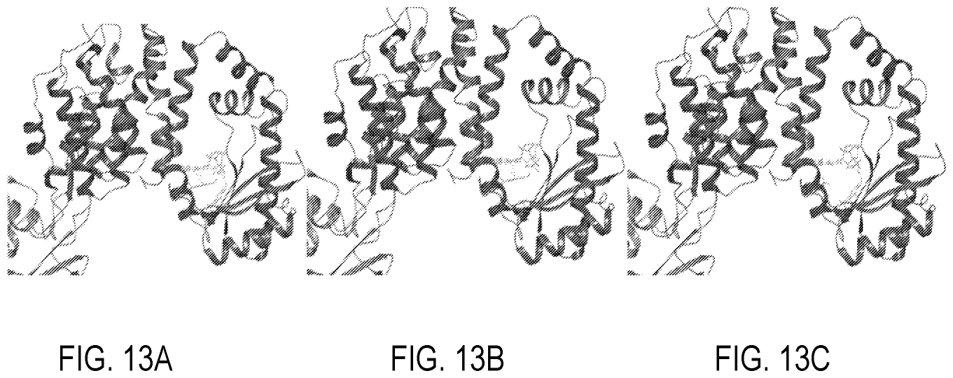

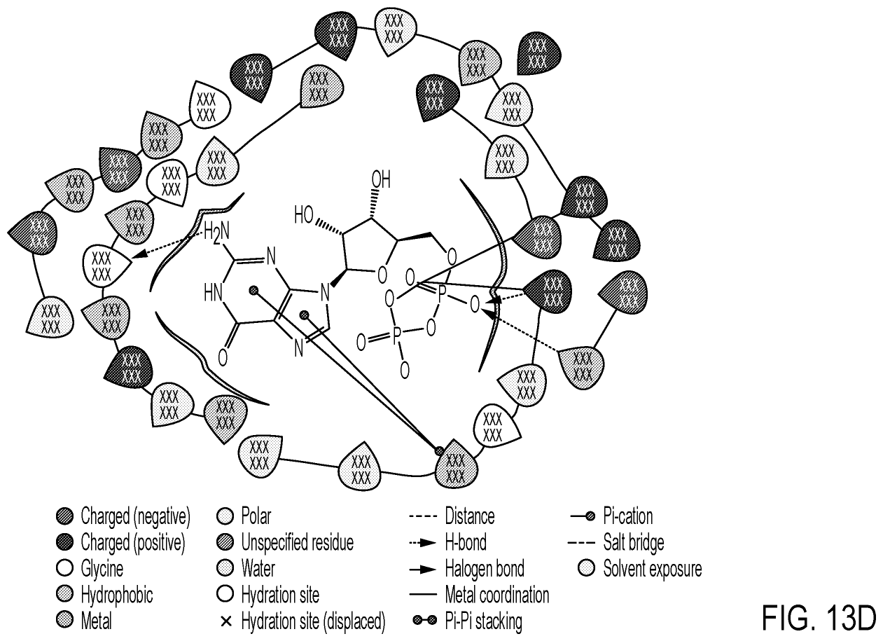

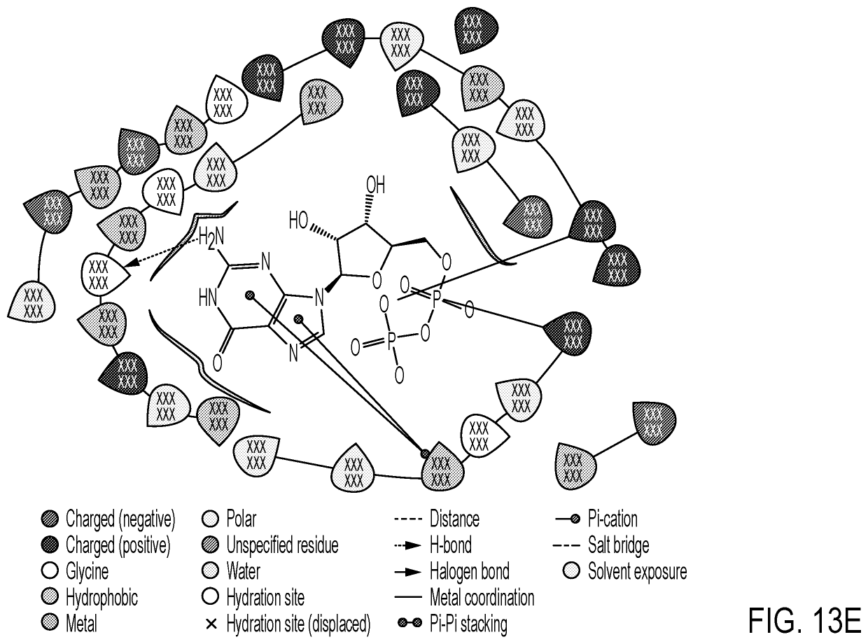

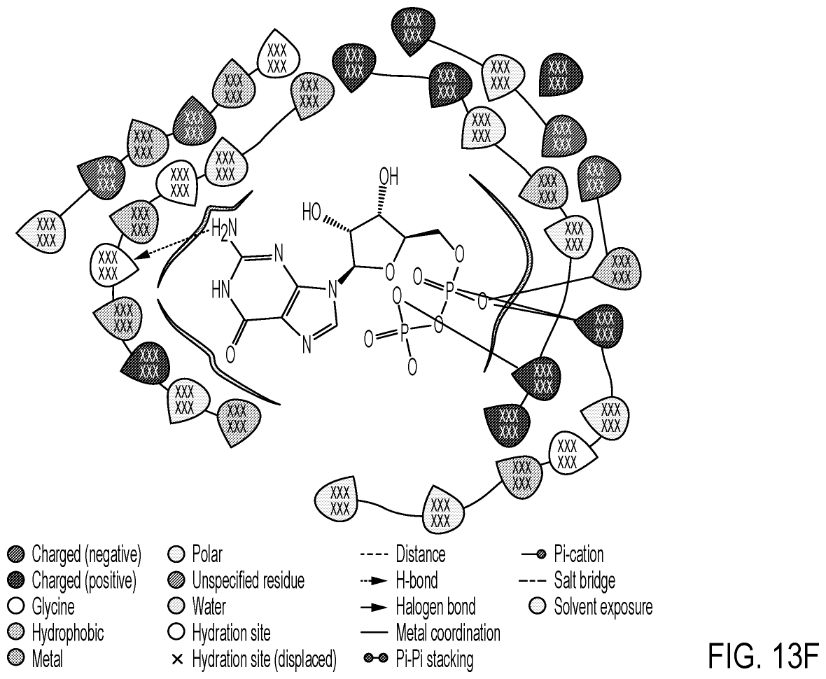

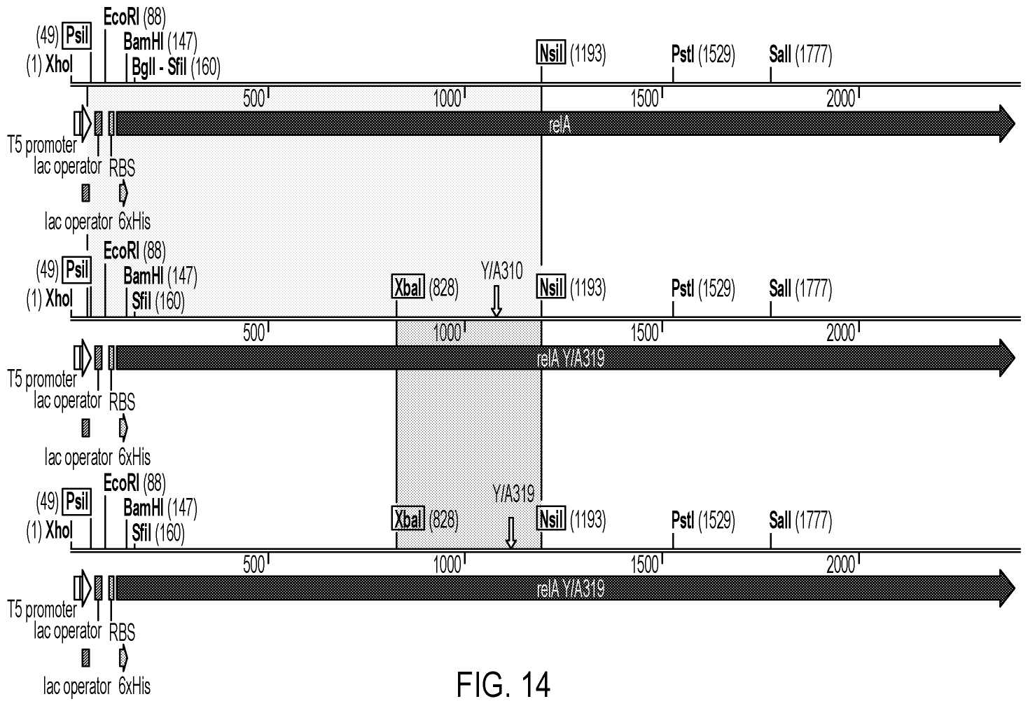

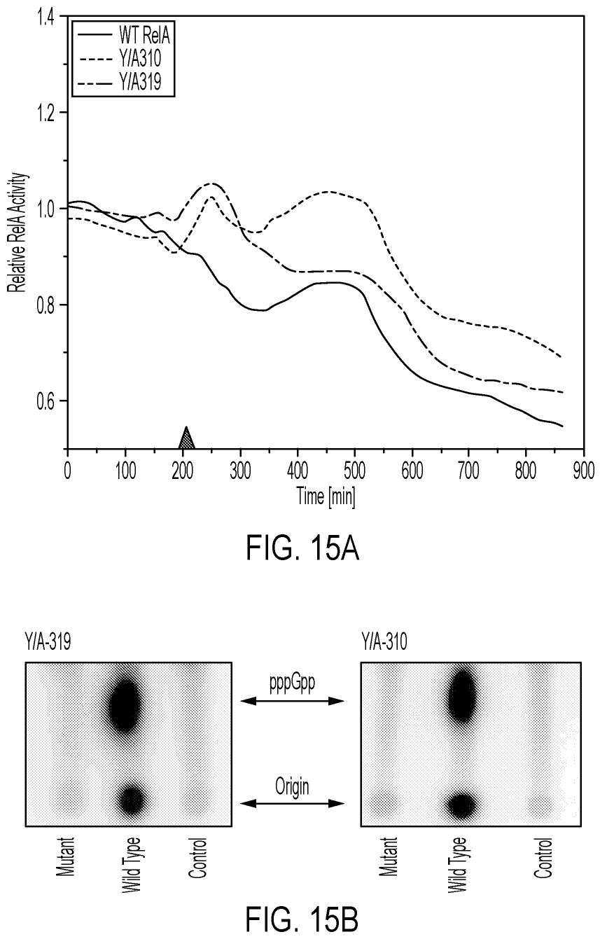

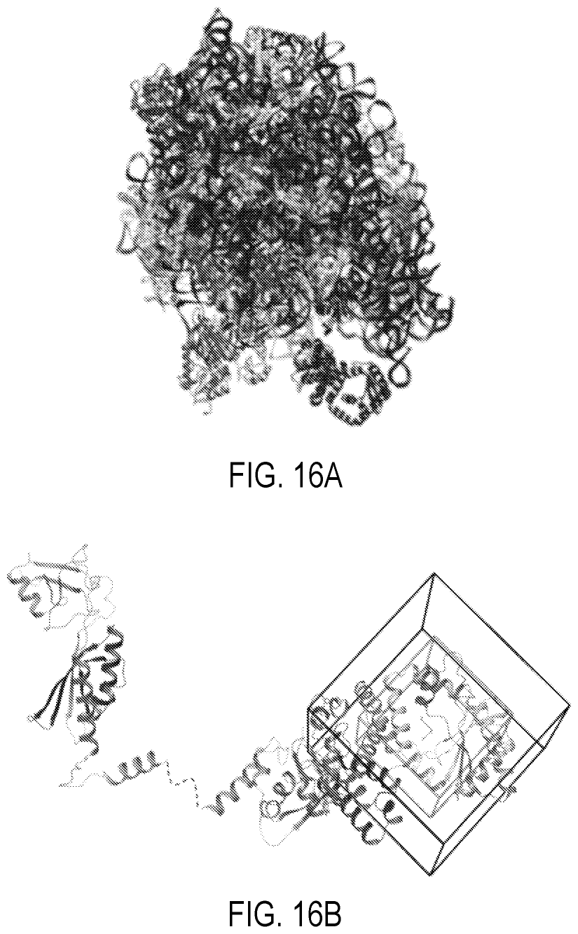

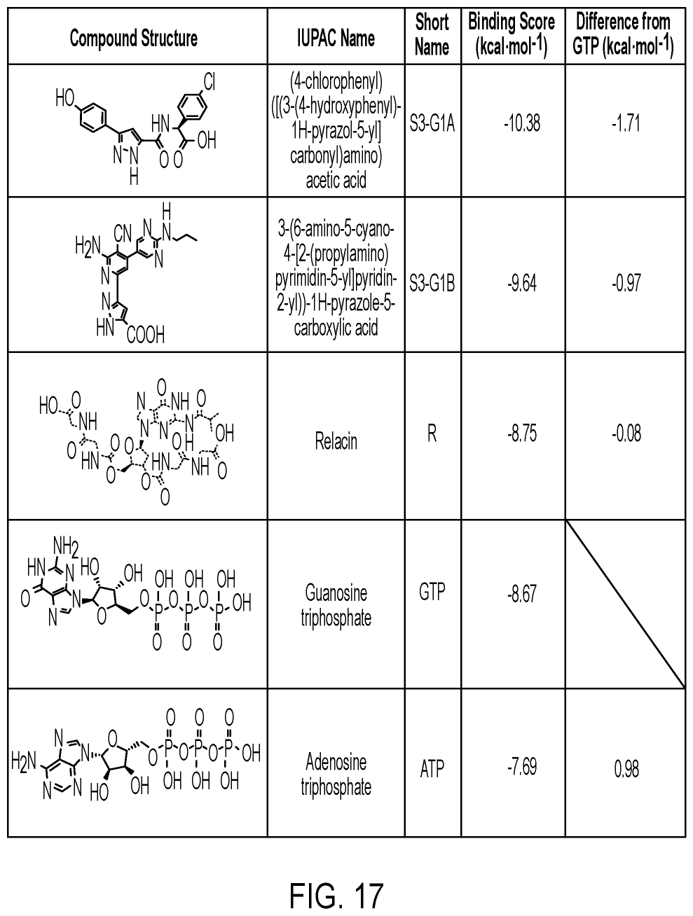

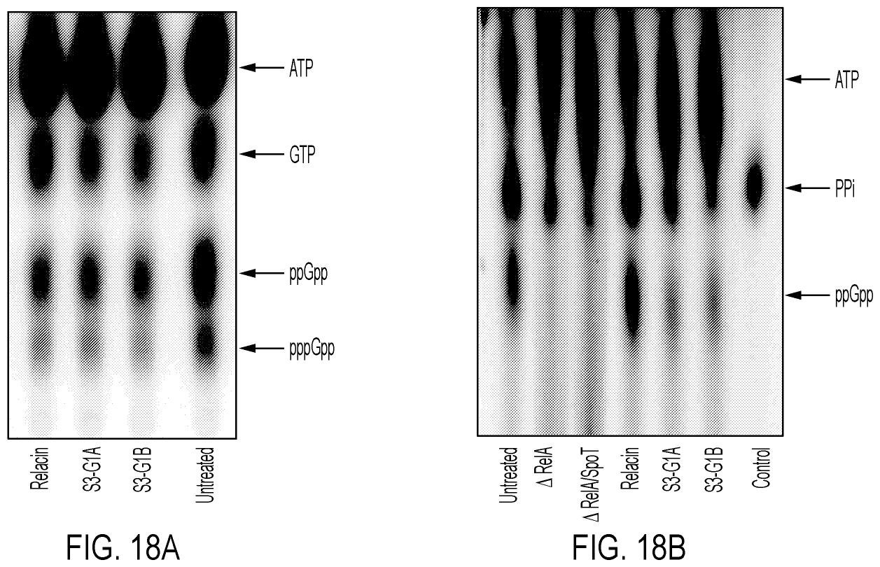

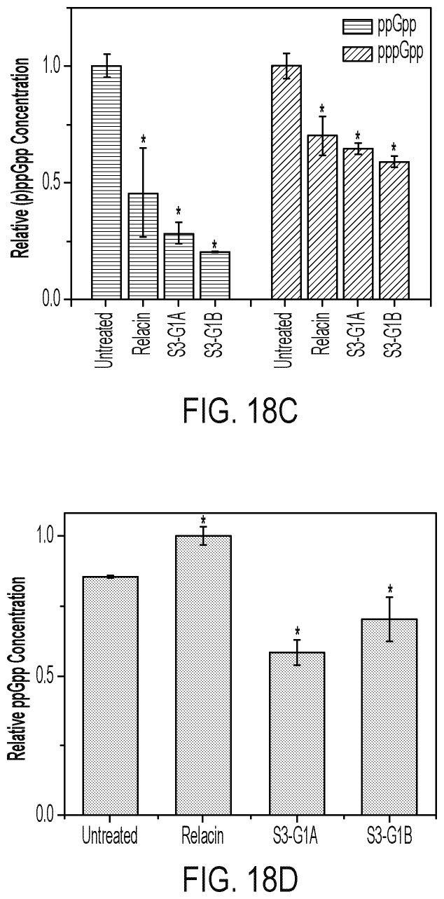

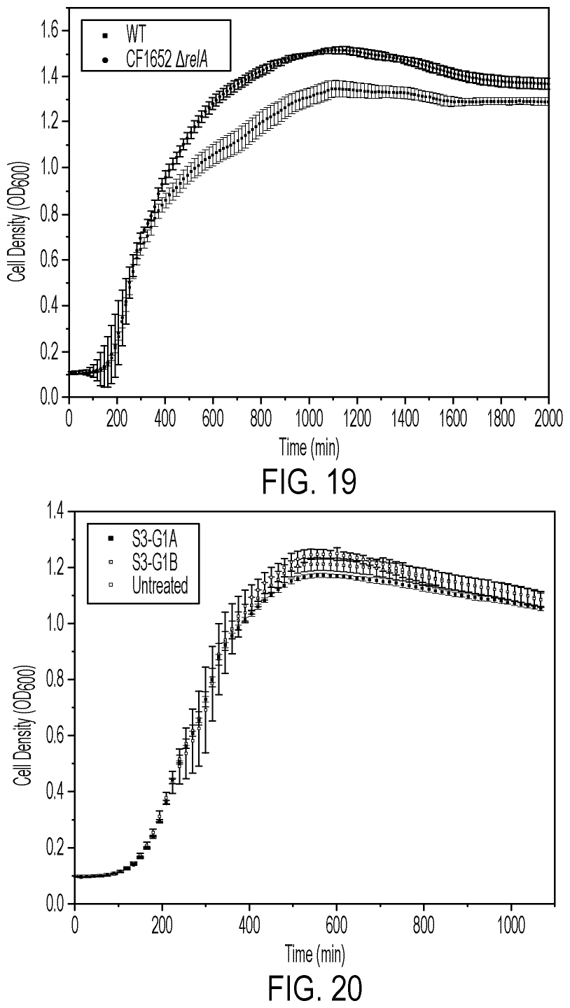

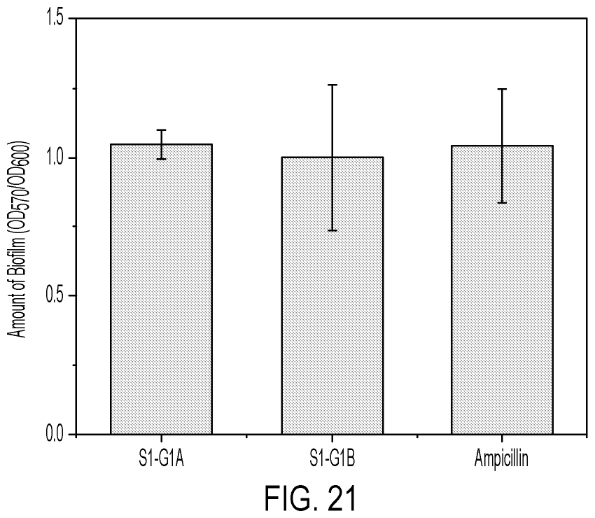

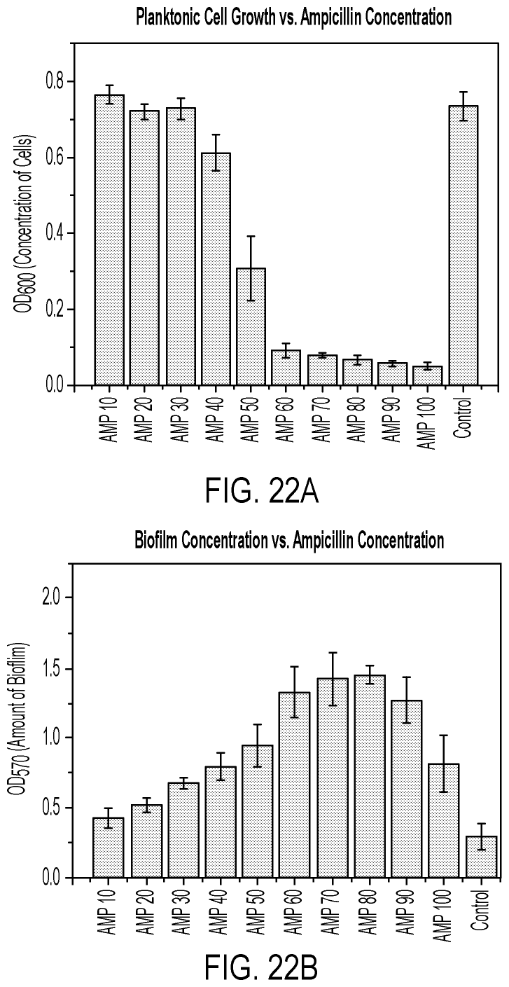

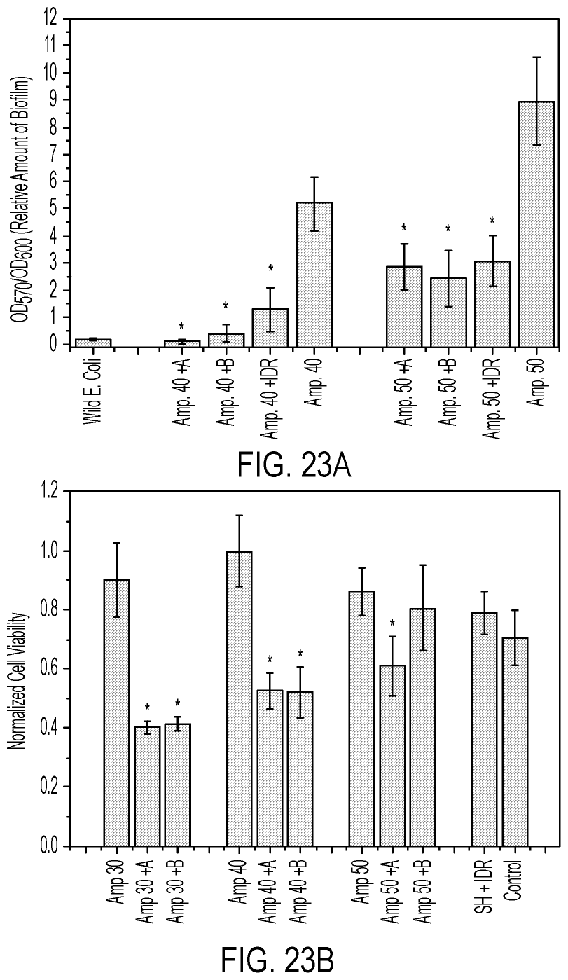

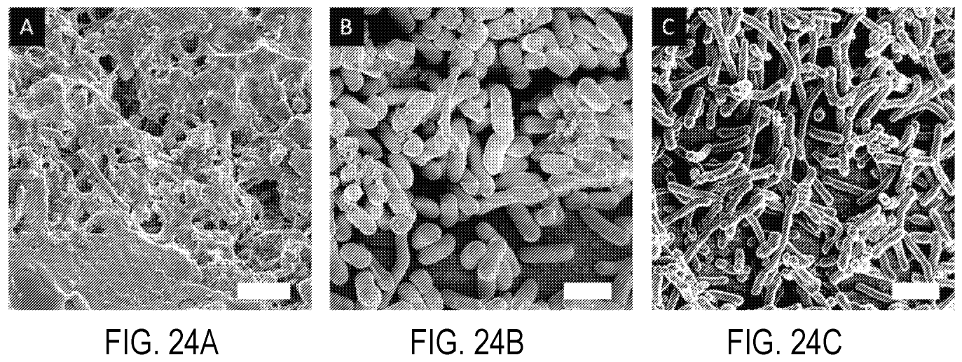

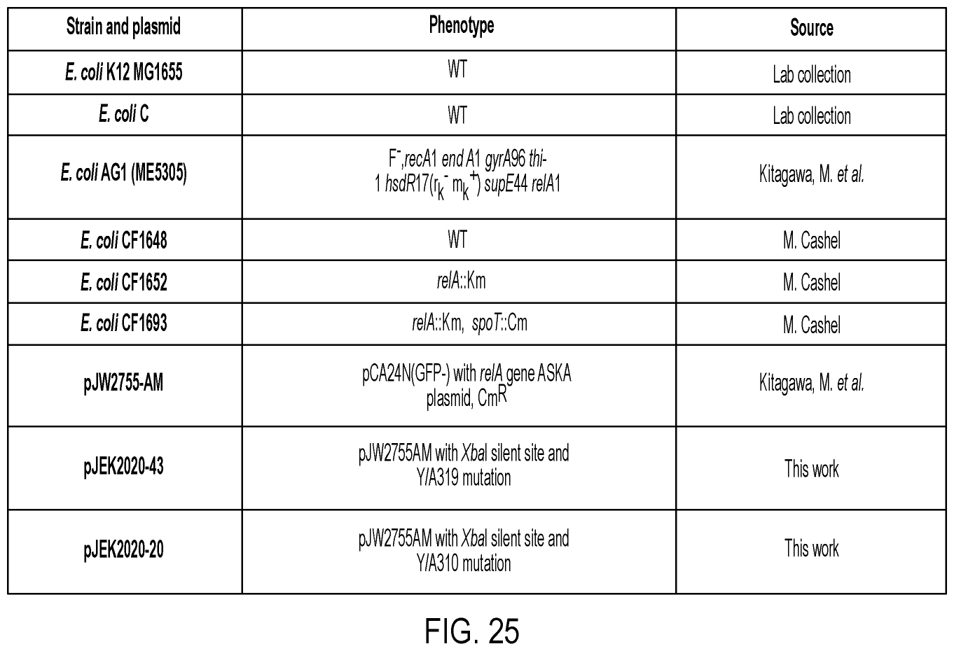

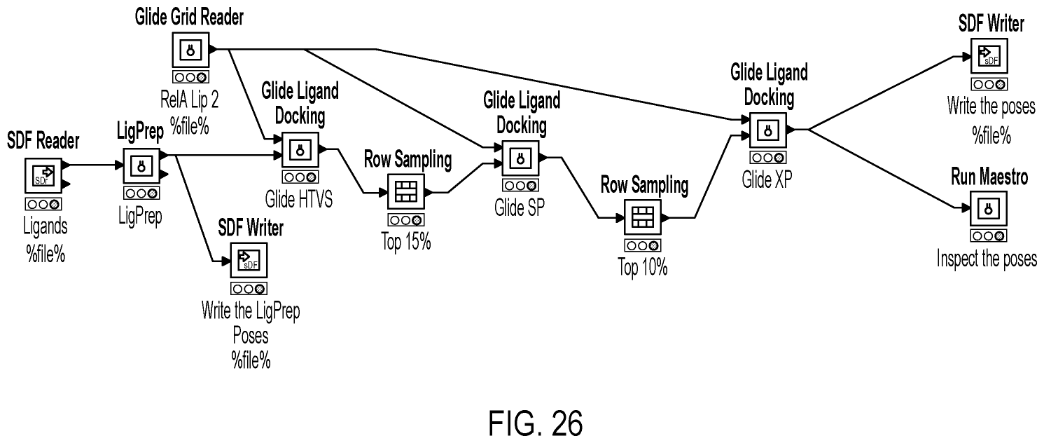

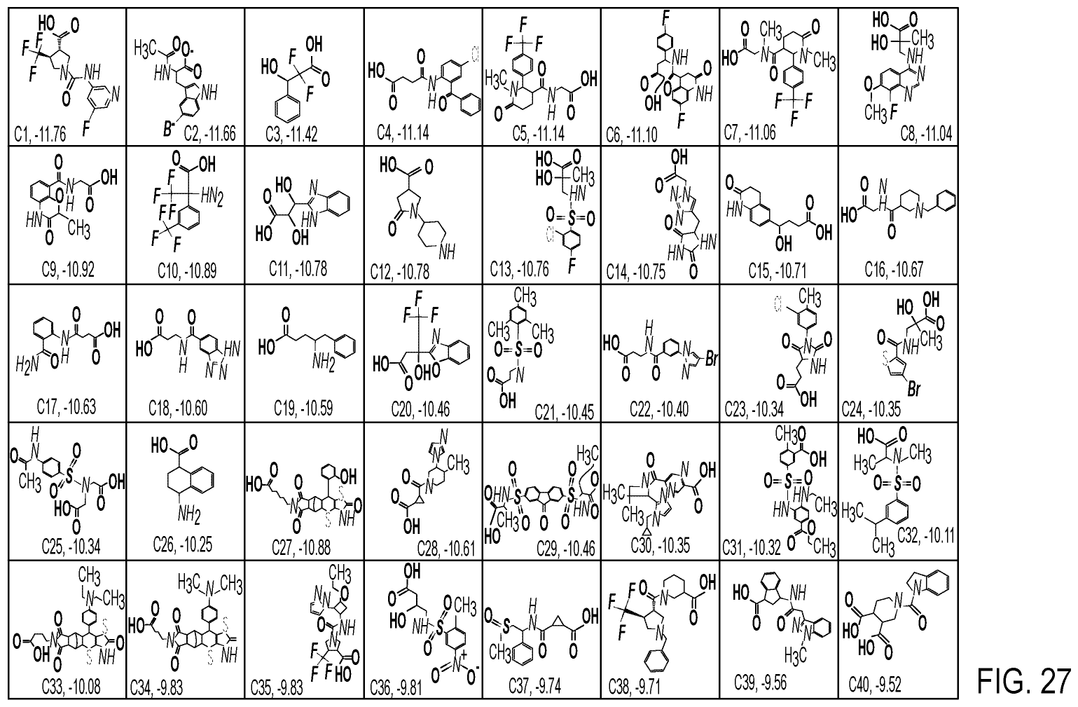

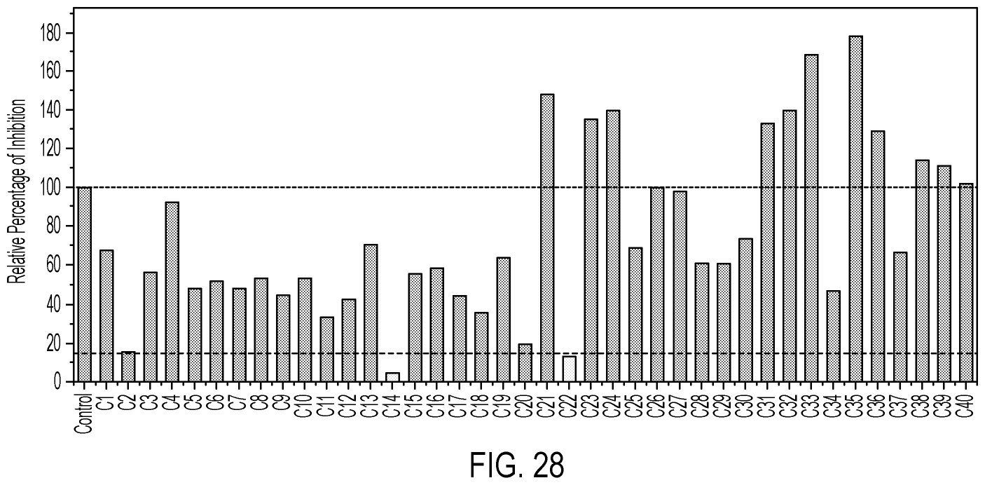

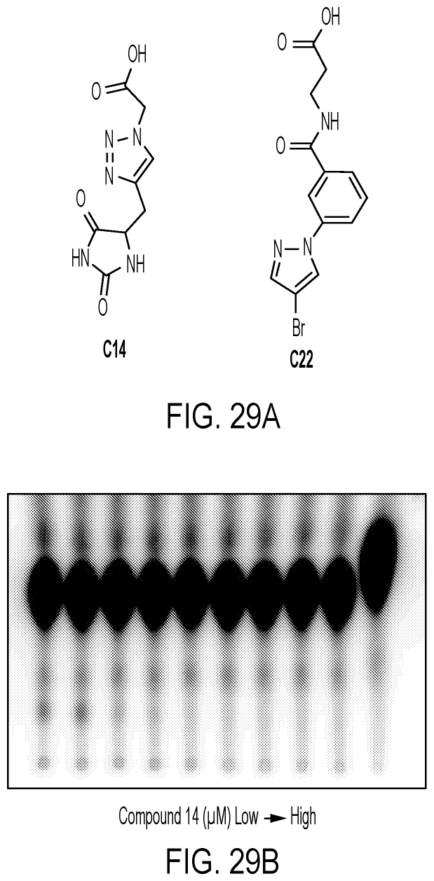

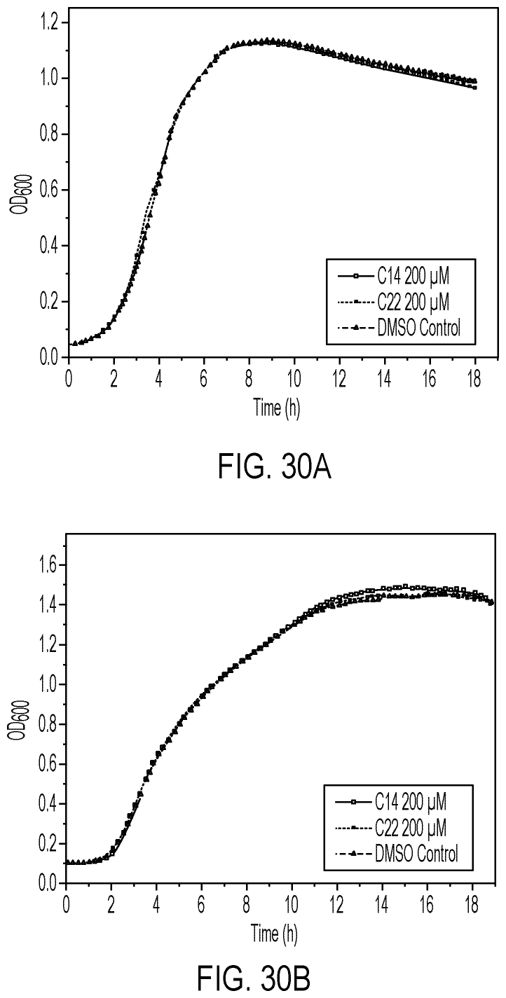

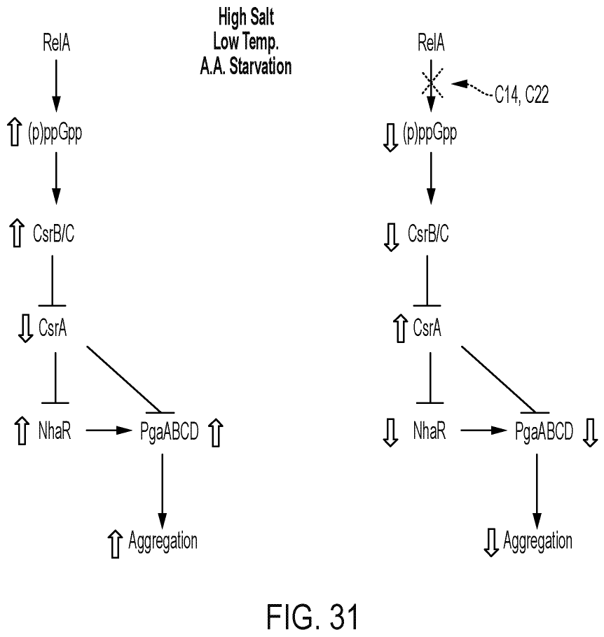

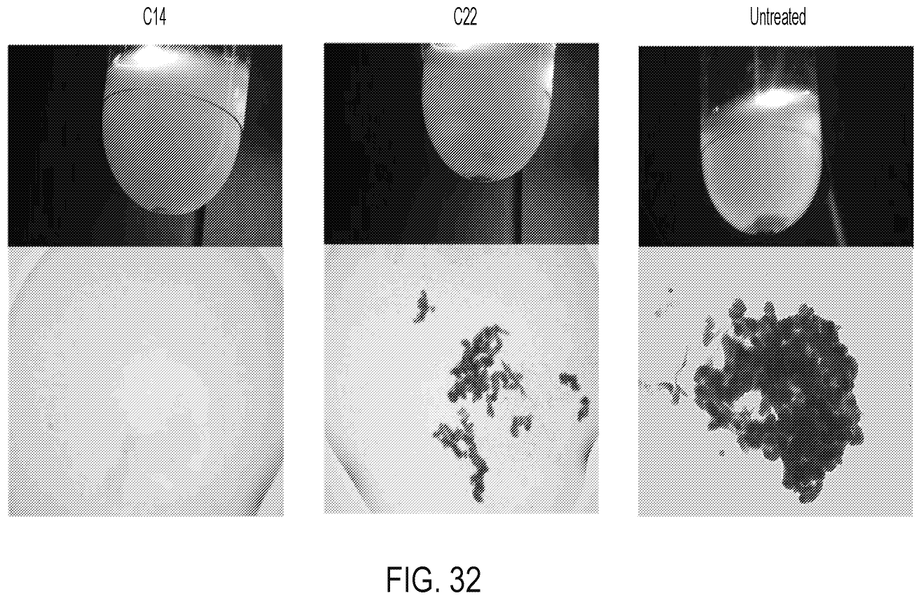

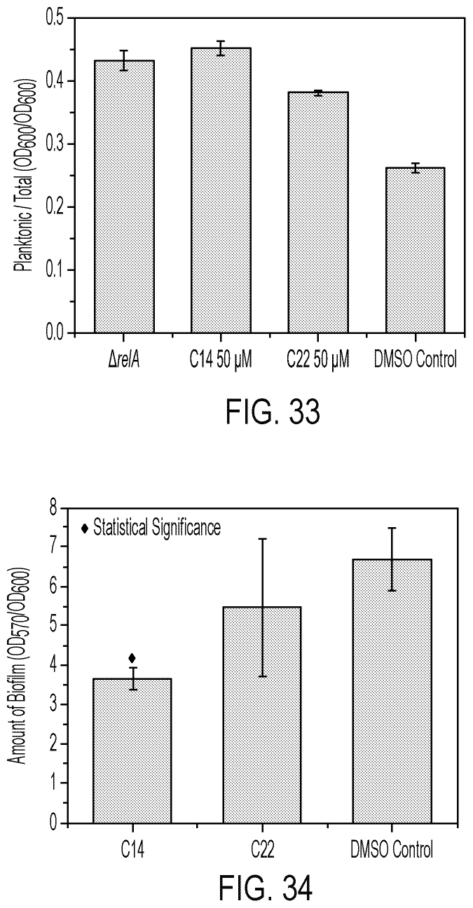

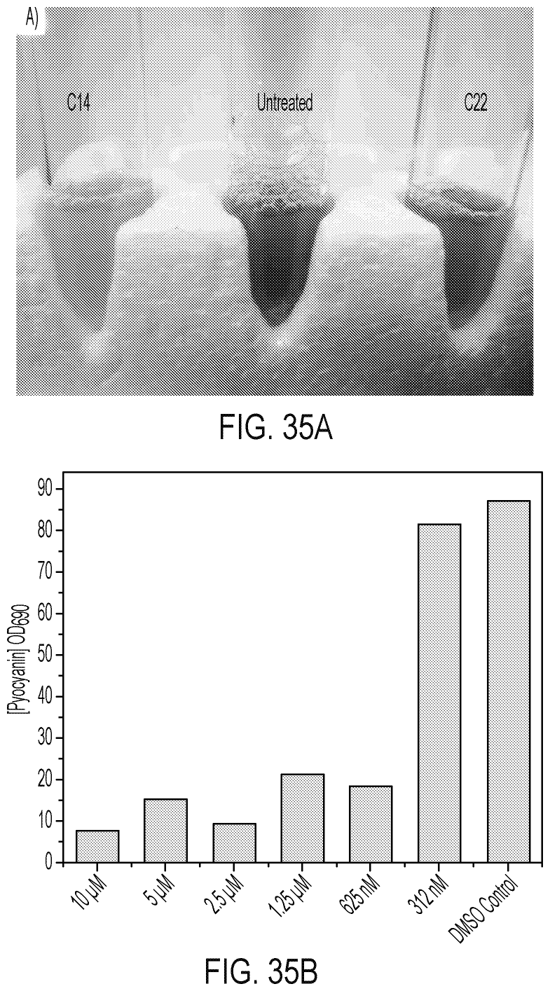

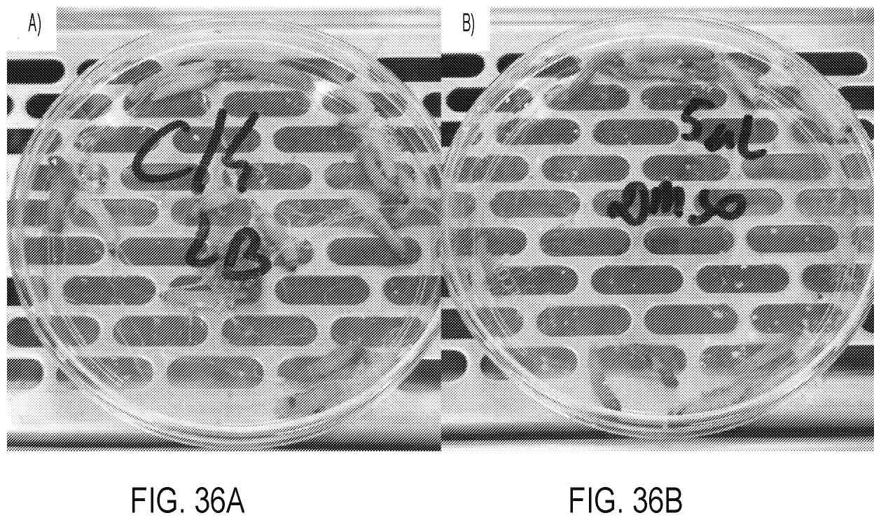

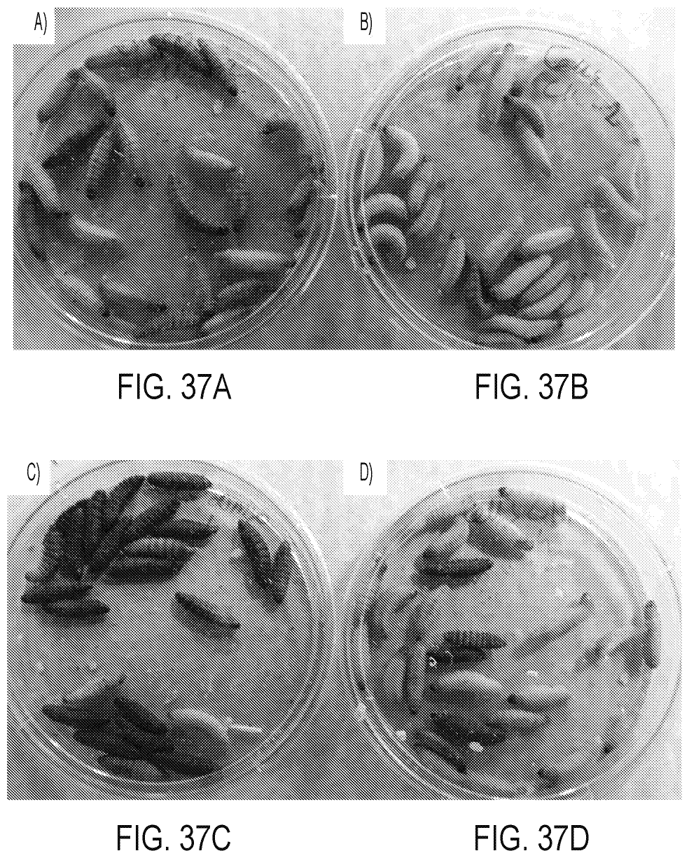

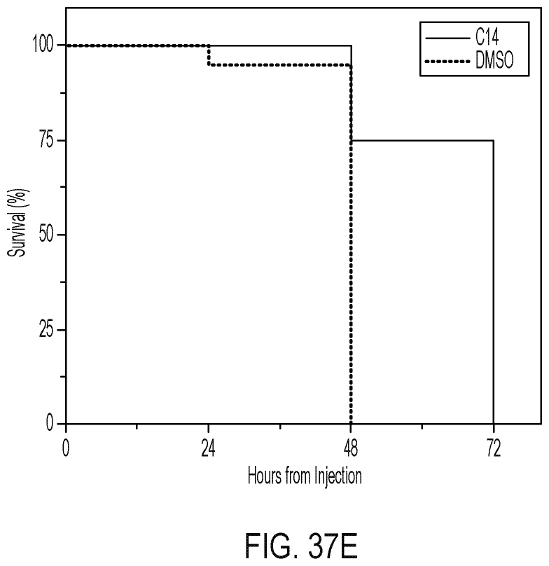

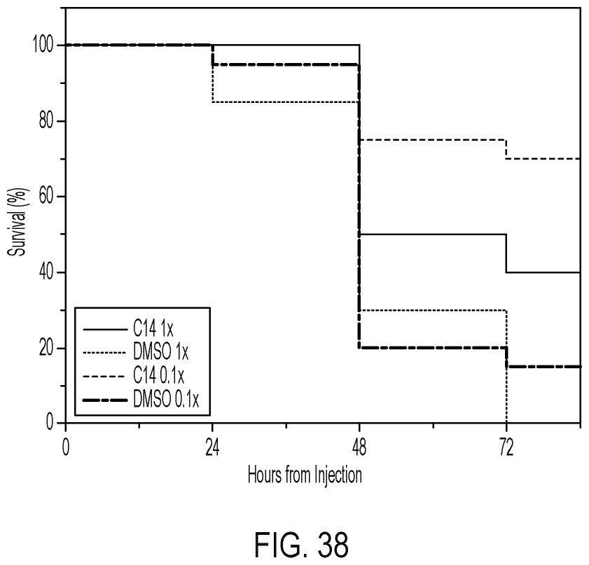

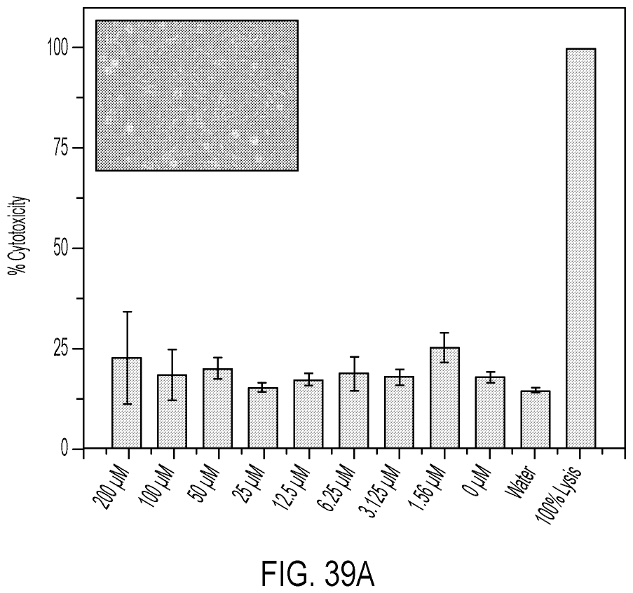

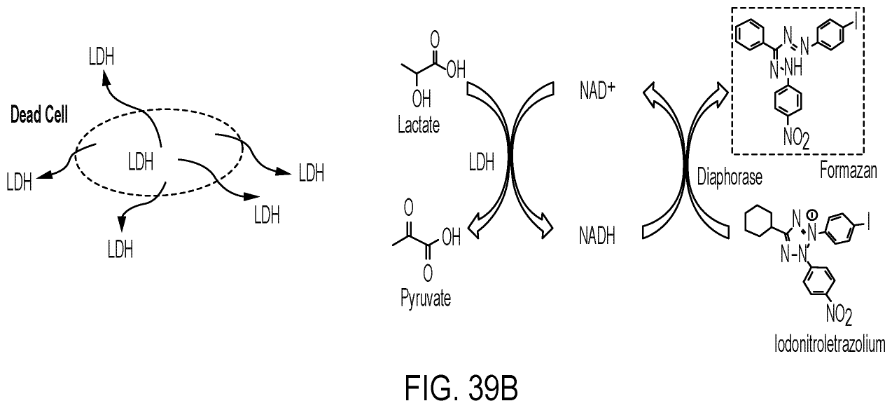

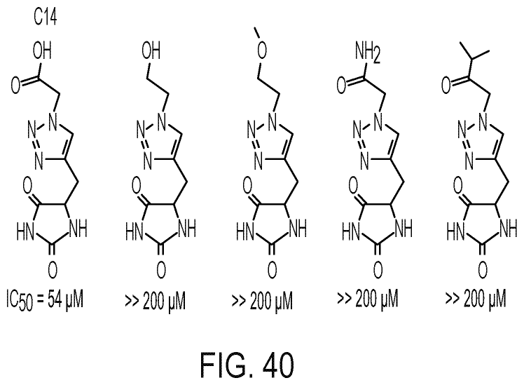

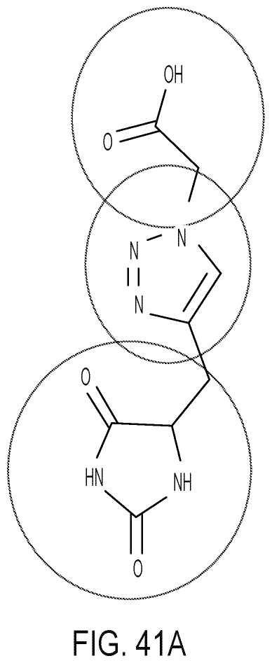

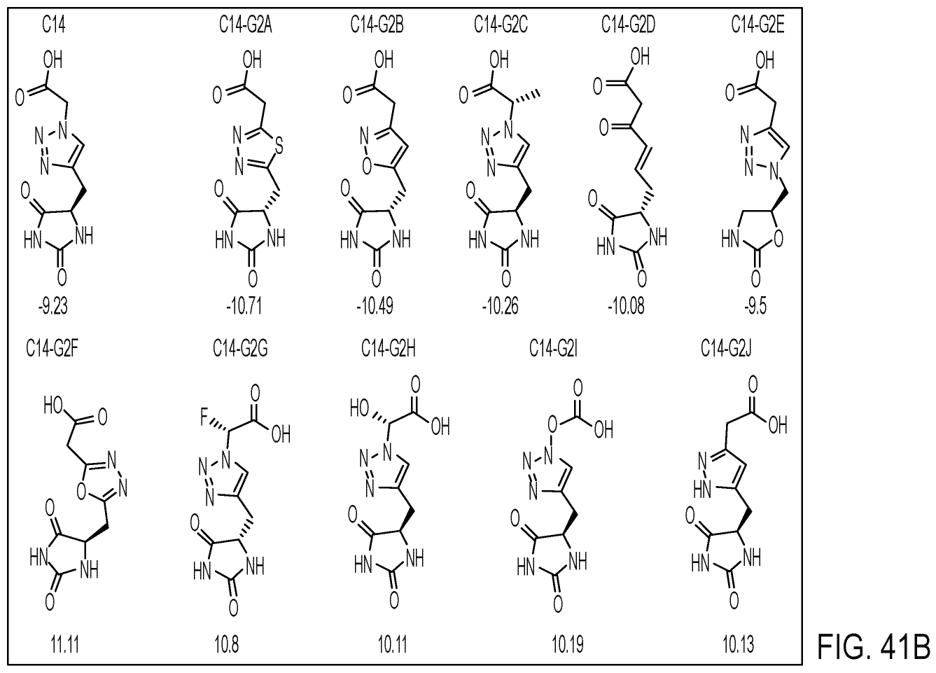

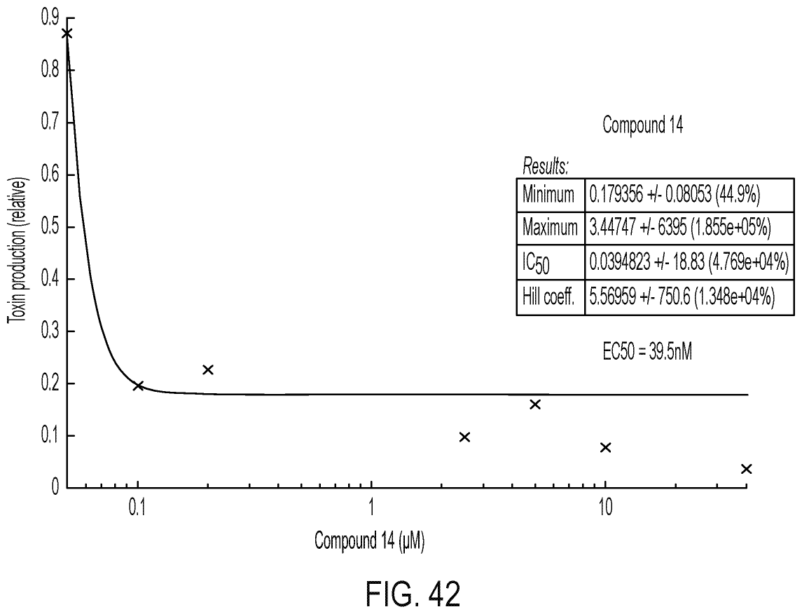

The active domain of the E. coli RelA cryo-EM (PDB: 5IQR) structure was determined using homology studies (FIGS. 12A-12D). Because there was no substrate bound to the RelA enzyme in the cryo-EM structure, the present study utilized two methods to determine the active site for molecular docking. The first method was a genomic-based homology method, where the known RelA protein sequences were compared, and the conserved residues were evaluated (FIG. 12D).

The second method was a structural homology method in which crystallographic data obtained from the S. aureus RSH-RelP that had been co-crystallized with its nucleotide substrates was used to identify both the pre- and post-catalytic active sites. Alignment of the RelA and RelP predicted active site residues showed that they are, structurally, highly similar; this allowed identification and characterization of the active domain for targeting via ligand docking studies (FIG. 12A). Using this information, the present study was able to determine two key amino acids involved in the binding of the first substrate in the catalytic process of GDP.

### Example 2-4: RelA Active Site Mutation Studies

To determine the accuracy of the in silico homology alignments and binding site determinations, two amino acid residues were identified as likely key to the catalytic activity of RelA, and then tested in the laboratory to ensure their assignment was correct (FIGS. 12A-12D). Tyrosine residues Y-324 and Y-332 (from the alignment) (FIG. 12D) had been determined to act as one of the largest contributors to the initial binding of GDP or GTP14. Y-324 was predicted to stabilize the phosphate of GDP/GTP by hydrogen bonding though means of its hydroxyl group; and Y-332 was predicted to be involved in π-stacking with the guanine's aromatic ring. These stabilizations were predicted to allow for the initial binding of GDP/GTP within the active site. Y-324 and Y-332 residues correspond to the Y-310 and Y-319 residues of the E. coli RelA enzyme. It was hypothesized that if these residues were mutated to alanines (A-310 and A-319) this should bring about a decrease in the catalytic transfer of the pyrophosphate from ATP to form (p)ppGpp.

FIGS. 13A-13F show the interactions of RelA with the native residues, as well as the lack of interactions when mutated to an alanine residue. To obtain the Y/A-310 and Y/A-319 substitutions of the E. coli RelA, the present study used two synthetic DNA cassettes to replace the 5′ end of the gene in the pJW2755-AM plasmid. The first PsiI/NsiI cassette (1144 bp) contained a silent XbaI mutation 30-31 and the Y/A-310 substitution. The second, XbaI/NsiI cassette (365 bp) introduced the Y/A-319 mutation (FIG. 14). The 365-bp region between the XbaI (769) and NsiI (1144) restriction sites contained the predicted RelA active center and can be easily exchanged with a synthetic construct to replace any of the tested amino acids.

Two assays were conducted to evaluate the activity of the mutant RelA enzymes: an in vivo (p)ppGpp fluorescent reporter and in vitro (p)ppGpp production assay. The ASKA plasmid pJW2755-AM with the WT RelA protein and its Y/A-310 and Y/A319 versions were transformed into the E. coli AG1 strain containing a pAG001 plasmid, this plasmid contains a YFP gene expressed under a stringent response regulated promoter PrpsJ.27 The E. coli AG1 strain contains a relA1 mutation caused by an insertion of an IS2 insertion sequence between the 85th and 86th codons of the relA gene. These mutants retain a low level of (p)ppGpp synthesis activity. Plasmids pJW2755AM, and its derivatives, and pAG001 belong to different incompatibility groups and therefore can co-reside in a single cell. When plasmid encoded RelA expression is induced with isopropyl β-D-1-thiogalactopyranoside (IPTG), the cells produce (p)ppGpp. Increased level of (p)ppGpp decreased the level of YPF synthesis, as it is under the control of the PrpsJ promoter. The results showed a much higher reduction of YFP fluorescence in the case of the WT RelA protein than with its Y/A-310 and Y/A-319 derivatives (FIG. 15A). In the in vitro assays the purified proteins containing the Y/A-310 and Y/A-319 when compared with the WT protein showed an even more striking reduction in pppGpp production (FIG. 15B). These results confirmed that the Y-310 and Y-319 amino acid residues play important roles in the enzymatic activity of RelA. Thus, the active site, as modeled elsewhere herein, can be used as a target for the in silico docking of ligands for the identification of candidate druggable inhibitors.

### Example 2-5: In Silico Screening for Hit Compounds

Non-RelA components of the E. coli RelA cryo-EM (PDB: 5IQR) model, including RNA and ribosome, were stripped away from the file leaving only the RelA structure (FIGS. 16A-16B). The RelA structure was then optimized using the Schrödinger Maestro protein preparation tools including the package Prime, which allows Maestro to fill in missing side chains and determine optimal amino acid orientations. The RelA enzyme was then structurally minimized using the force field OPLS3e33 (FIG. 16B). The enzyme binding pocket was determined using homology studies (FIGS. 13A-13F), as well as a general understanding of RelA's function, and a docking grid box was developed for protein ligand docking calculations.

Schrödinger Maestro Molecular Modeling Glide was utilized to determine hit compounds, which were then validated using the laboratory assays described elsewhere herein to probe their ability to inhibit RelA activity. Schrödinger Glide-HTVS mode was first used to screen the entire University of California, San Francisco Zinc Database of commercially available compounds. This database contains over 4 million compounds. The top 10% from the HTVS docking scan was then filtered into Glide-SP mode (standard precision). This output was then further refined and run in Glide-XP35 mode (extra precision). These molecular docking studies resulted in 2 compounds showing a binding score that passed the threshold for binding affinity (FIG. 17) and were higher than those of the natural substrates ATP and GTP. These two compounds also fit both the Lipinski's rule of 5 for orally administered drugs, and the quantitative estimate of drug likeness.

### Example 2-6: Effect of S3-G1A and S3-G1B on (p)ppGpp Production Via In Vitro and In Vivo RelA Assays

After computational hit compounds were determined, the next step was to evaluate the effect of these small molecules on RelA activity in the in vitro and in vivo assays established elsewhere herein for the production of ppGpp. The results of the in vitro assay showed that both compounds S3-G1A (20 μM) and S3-GIB (20 μM) reduced the ppGpp production when compared to an untreated sample by 71.7% (p<0.0001) and 79.7% (p<0001), respectively (FIG. 18A). Both compounds showed higher reduction of activity than Relacin (45.4%, p=0.0084). The in vivo assay showed a reduction in ppGpp production in samples treated with both compounds 31.4% (p=0.0006) in S3-G1A and 17.75% (p=0.0295) in S3-GIB. In this assay, no effect of Relacin on ppGpp production was observed (FIG. 18B). It was hypothesized that Relacin is not cell permeable and therefore does not influence the in vivo ppGpp production. These results indicated that the S3-G1A and S3-GIB compounds are more efficient in vivo and in vitro than Relacin and validated the entire hybrid in silico-laboratory pipeline.

### Example 2-7: Effect of Hit Compounds on Bacterial Growth

Bacterial growth rate under conditions unrestricted by substrate availability is an indicator of cell health and viability. Despite great efforts to determine the role of the stringent response on control of cell growth rate, general conclusions have not been able to be drawn. However, all reports have shown that mutants unable to produce ppGpp grow slightly more slowly (up to 30%) than their cognate WT on all media tested. It was found that the initial growth rates for the WT strain and CF₁₆₅₂ (relA::Km) were the same (FIG. 19).

However, growth of the WT strain started to slow down first after reaching OD₆₀₀=0.6. The WT strain was expected to sense small changes in nutrient concentrations and react to it, reducing the growth rate. The relA mutant reached a higher cell density than that of the WT. After 18 h of growth, both strains reached their highest cell densities and thereafter varying decreases in OD₆₀₀ values were observed. It was found that compounds S3-G1A and S3-GIB had no effect on planktonic growth rate. The maximal cell densities of the cultures with compounds were slightly lower than the control (FIG. 20).

### Example 2-8: Effect of Hit Compounds on Biofilm Inhibition and Dispersal

It was previously reported that E. coli strain C is the only one of the five major “laboratory strains” of E. coli that is a superior biofilm former; therefore, this strain was used in the biofilm assays. Studies were conducted in 96-well high-throughput assays. In the biofilm inhibition assay, compounds were added to the wells at the beginning of the experiment. For the biofilm dispersal assay, the biofilm was allowed to grow for 24 and 48 h, the wells were washed with sterile phosphate-buffered saline (PBS), and fresh medium supplemented with the compounds was added to the wells. The amount of biofilm was measured after 24 h. There was no observed effect on the inhibition (FIG. 21) or dispersal (data not shown) of biofilms with compounds alone.

### Example 2-9: Effect of Compound on Biofilm Persistence and Biofilm Viability

Biofilm persistence and viability were assessed with the hit compounds in combination with an antibiotic. It has been determined that sub-MICs (minimum inhibitory concentration) of antibiotic result in increased biofilm formation. Ampicillin was used in all of the assays due to its bactericidal effect. Sub-MIC concentrations of ampicillin were determined by growth measurements (OD₆₀₀). It was found that the biggest change in the culture cell density was observed between 40 and 60 μg·mL⁻¹ ampicillin (FIGS. 22A-22B). Analyzing the effect of ampicillin on biofilm formation, it was observed that the presence of the antibiotic significantly increased the amount of biofilm with the highest biomass observed at relatively high ampicillin F concentrations (80 μg·mL⁻¹) (FIG. 22B). To analyze the effect of the hit compounds in combination with antibiotics, a range of ampicillin concentrations, from 30 to 50 μg·mL⁻¹, were utilized.

The amount of biofilm biomass was determined in the combined presence of antibiotics and either compound A & B. This combination therapy led to a highly significant reductions in biofilm mass compared to the ampicillin-only-treated controls (FIG. 23A). As a reference control, the present study used IDR 1018, an antimicrobial peptide that was reported to target (p)ppGpp directly and degrade ppGpp in vitro. Addition of the hit compounds to ampicillin concentrations of 40 μg·mL⁻¹ (Amp40) and 50 μg·mL⁻¹ (Amp50) resulted in a highly significant decrease in biofilm volume compared with their cognate antibiotic only control (FIG. 23A). At Amp40 the biofilm biomass was reduced by 97.9% (p=0.0009) for S3-G1A (50 μM), by 92.4% (p=0.0014) for S3-G1B (50 μM), and by 75.4% (p=0.006) for IDR1018 (5 μM). Amp50 showed reductions in biofilm biomass of 67.9% (p=0.0044) for S3-G1A, of 72.9% (p=0.0042) for S3-G1B, and 65.2% (p=0.0054) for IDR1018. The difference between Amp40 and Amp50 can be attributed to the greater increase in biofilm volume induced by the higher, but still sub-MIC concentration of the antibiotic.

An AlamarBlue cell viability assay also showed that ampicillin killed more bacterial cells in combination with the tested hit compounds (FIG. 23B). In the case of ampicillin at 30 μg·mL⁻¹ (Amp30), the reduction was 55.4% (p=0.0024) and 54.2% (p=0.0027) for S3-G1A (50 μM) and S3-61B (50 μM), respectively. When higher concentrations of antibiotic were used, the synergetic effects of compounds S3-G1A and S3-GIB were less noticeable, with the decreases being only 29.2% (p=0.0278) and 6.5% (p=0.6), respectively. This effect was attributed to the greater volume of the biofilm contained in these samples (FIG. 23B).

### Example 2-10: Effect of Hit Compounds on Biofilm Structure

Scanning electron microscopy (SEM) allowed us to probe the structure of the biofilms treated with the hit compounds. Biofilms were grown on metal pins for 3 days that were transferred daily to fresh LB medium using the JEKMag technique. While there was not a large reduction in biofilm mass by the compounds alone, there was a very substantial change to the structure of the extracellular matrix of the biofilms. Biofilms treated with 40 μg·mL⁻¹ of compound S3-G1A and 40 μg·mL⁻¹ of compound S3-GIB exhibited a greatly reduced amount of extracellular matrix compared to untreated WT E. coli C (FIGS. 24A-24C). Treatment with S3-GIB also resulted in elongation of the cells, indicating the possibility of an unknown off-target effect inducing filamentation.

### Example 2-11

The present study has established a hybrid in silico-laboratory pipeline method to identify and characterize novel RelA inhibitors for the treatment of medically relevant bacterial biofilms in combination with traditional antibiotics. Using these reported methods in combination has given us the ability to determine and characterize hit compounds from a large database of in silico ligands. These methods have provided us with two lead compounds that are being utilized in downstream optimization structure-activity relationships to improve the efficacy of the core bioisostere. The methods outlined here are important steps toward the process of finding an effective inhibitor of the RelA-driven bacterial stringent response and, in turn, the treatment of persistent biofilm infections. The computational components, which include binding-site determinations and a multi-step docking process, that incorporates a series of every more rigorous filters, provides for the efficient screening of large ligand libraries, and provides an effective and cost-effective means for identifying hit molecules for the inhibition of RelA. Before the addition of these in silico methods, high-throughput ligand assays in the biofilm space have been costly and time consuming.

### Example 2-12: Experimental

### Bacterial Strains and Growth Conditions

Bacterial strains are listed in FIG. 25. All bacterial strains were grown in Luria-Bertani broth (LB) or LB agar (1.5%). Antibiotics kanamycin (50 μg·mL⁻¹), ampicillin (100 μg·mL⁻¹), and chloramphenicol (25 μg·mL⁻¹) were used when necessary. S3-G1A was synthesized in house and S3-G1B46 was purchased from Hit2Lead and used at the concentrations described herein.

### Computational Docking

High-throughput in Silico Docking Studies: The RelA enzyme (PDB: 5IQR) was prepared and optimized using Maestro Protein Preparation (Schrödinger Maestro, New York, NY, USA; Version 11.9.011, MMshare Version 4.5.011, Release 2019-1, Platform Windows-x64). The 5IQR PDB file contained extraneous portions of the ribosome, as the structure was determined as a RelA dimer with the ribosome. The ribosome and RNA subunits were removed and RelA was isolated in a separate file. The dockable RelA structure was prepared and minimized using Schrödinger's protein preparation application. This application was utilized to add hydrogens, create missing disulfide bonds, and determine lowest-energy residue orientations. Geometry minimization was carried out using the force field OPLS3e33. A docking site was determined using homology studies of bacterial rel genes from several species in combination with the Schrödinger binding site determination tool. Ligands were prepared using Schrödinger LigPrep (Schrödinger Release 2020-1: LigPrep, Schrödinger, LLC, New York, NY, USA, 2020).

### Biological Validation Assays

RelA Mutagenesis: The ASKA(−) clone JW2755-AM containing an E. coli W3110 RelA in the pCA24N vector was used for mutagenesis. A 1144-bp PsiI/NsiI fragment was replaced by a synthetic construct. This construct contained 2 designated changes. First, a single nucleotide silent substitution (769 C/A) introduced an XbaI restriction site. Second, a TA/GC substitution at position 1027 replaced TAC (Y-310) with GCC (A-310). The 365-bp region between XbaI (769) and NsiI (1144) contains the RelA active center and can be easily swapped with a synthetic construct to replace any of the tested amino acids. This method was applied to introduce the Y/A-319 mutation. A 365-bp XbaI/NsiI fragment was replaced by a synthetic fragment with TAT-Y319 (position 1053) replaced with GCC-A319 codon as described48. All mutations were confirmed by Sanger DNA sequencing.

RelA Protein Purification: the functional RelA enzyme and its Y/A-319 and Y/A-310 mutants were purified from host cell AG1 strains carrying the pJW2755-AM, pJEK2020-43, and pJEK2020-20 plasmids, respectively. One liter of LB broth was inoculated with 20 mL of overnight culture (OD₆₀₀=0.9) and grown for 4 h (OD₆₀₀=0.8) before induction with 1.5 mM IPTG for 4 h. Cultures were spun down, washed with phosphate-buffered saline (PBS), and resuspended in lysis buffer (50 mM NaH₂PO₄, 300 mM NaCl, 10 mM imidazole, pH 8.0) for lysing. To that resuspension, 1 μL·mL⁻¹ ThermoFisher Halt™ Protease Inhibitor Cocktail (100×) was added without EDTA and cells were lysed with sonication on ice (cycles 10 s on 10 s off for a total of 3 min of sonication, 2×). Lysates were spun down to remove cellular debris. Millipore Sigma PureProteome™ Nickel Magnetic Beads were used according to modified manufacturer's instructions. Supernatant was placed in 200 μL of nickel affinity beads for a period of 30 mins. Beads were captured on a magnetic rack and the supernatant was removed. Beads were then washed 4× with wash buffer (50 mM NaH₂PO₄, 300 mM NaCl, 10 mM imidazole, pH 8.0). RelA was eluted twice using 300 mM imidazole elution buffer (50 mM NaH₂PO₄, 300 mM NaCl, 300 mM imidazole, pH 8.0) and a final elution using 500 mM imidazole elution buffer (50 mM NaH₂PO₄, 300 mM NaCl, 500 mM imidazole, pH 8.0). An SDS-PAGE gel was run to confirm presence and purity of RelA. Imidazole buffer was exchanged for PBS buffer and RelA was concentrated using Amicon® Ultra-4 Centrifugal Filter Unit 30 KDa nominal molecular weight limit. Nanodrop showed an average concentration of 1 mg mL⁻¹ with a 260:280 ratio ˜ 0.73.

Fluorescent Reporter RelA Activity Assay: The plasmid pAG001 (ampicillin 100 μg·mL⁻¹) carrying a yfp fluorescent protein gene driven by the PrpsJ was used to detect the intracellular ppGpp concentrations. To validate the assay this reporter plasmid, which is based on the broad host range RK2 minimal replicon, it was introduced into E. coli K12 CF1648, and its relA mutants: (CF1652)32, and AG1 (relA1) (NBRP Japan). To analyze the effect of overexpression of RelA and the Y/A310, and Y/A319 substitutions, ASKA plasmid pJW2755-AM49 (chloramphenicol 25 μg·mL⁻¹) and its derivatives pJEK2020-20 with the Y/A310 mutation and pJEK2020-43 with the Y/A319 mutation were extracted using the ThermoFisher Plasmid Mini DNA Extraction Kit, and transformed into AG1pAG001 strain (ampicillin 100 μg·mL⁻¹, chloramphenicol 25 μg·mL⁻¹). For the fluorescent RelA activity assay, overnight cultures of the selected strains were diluted 1:100 in fresh LB medium and 200 μL aliquots were placed into 96-well plates (Costar). The plates were placed in a Tecan Infinite M200 Pro Microplate Reader with a programmed growth cycle (18 h, 37° C., orbital rotation 3.5). Cell density was measured at OD₆₀₀ and YFP fluorescence activity was detected with 505/535 nm (excitation/emission). Enzymatic activity was measured as Relative Fluorescence Units (RFU-YFP/OD₆₀₀).

In vitro (p)ppGpp quantification: in vitro (p)ppGpp quantification was carried out. RelA enzyme was purified as described elsewhere herein. Roughly 0.4 μg of RelA protein was added to a 1.5-mL microcentrifuge tube containing a reaction mix composed of 1×PBS, 5 mM MgCl₂, 0.5 mM ATP, 0.5 mM GTP, 0.5 mM GDP, and 20 μCi[γ-32P]ATP (3,000 Ci mmol-1; PerkinElmer) and varying concentrations of the compound of interest. These reactions were incubated at 37° C. for 1 h. The reactions were stopped by addition of 5 μL formic acid (88%). The reaction mixtures were then spotted on a stationary-phase polyethyleneimine (PEI)-cellulose TLC plate using potassium phosphate monobasic (1.5 M) as the mobile-phase. The plates were then dried, and the radiation levels were read using a Molecular Dynamics Storage Phosphor Screen. A Molecular Dynamics Storm 840 Phosphor imager Scanner was used to read the phosphor screen and ImageJ was used to process the images.

In vivo (p)ppGpp Quantification: In vivo (p)ppGpp quantification was carried out. One milliliter of overnight cell culture of E. coli C was placed in 1.5-mL microcentrifuge tubes and pelleted. To this pellet was added 50 μL of a reaction mixture containing 20 μCi orthophosphoric acid and 40 μM serine hydroxamate in 1×MOPS minimal medium. The cell pellet was resuspended by gentle vortexing and placed in an incubator for 1 h. Cell growth arrest and cell lysis were completed by addition of 15 μL formic acid (88%). The lysate was then centrifuged to remove any insoluble components and the supernatant was spotted on a stationary-phase PEI-cellulose TLC plate. Plates were processed and analyzed as described elsewhere herein.

Biofilm Dispersal Assays: For biofilm formation on polystyrene surfaces, flat-bottom 96-well microtiter plates (Corning Inc.) were used. Two hundred microliters of bacterial culture (100× diluted overnight culture; approximately 10⁷ cells) in fresh LB medium was added to each well. These were allowed to grow for 24 h. The planktonic cells and medium were then aspirated, and the plates were washed twice with 1×PBS. Fresh LB with hit compounds were added to the biofilm wells. These cultures were then allowed to incubate at 37° C. overnight. Cell density were measured (OD₆₀₀) using a Multiscan Go plate reader (ThermoFisher), and 30 μL Gram crystal violet (CV) (Remel; 3 g crystal violet, 50 mL isopropanol, 50 mL ethanol, 900 mL purified water) was applied for staining for 1 h. Plates were washed with water and air dried, and CV was solubilized with an ethanol:acetone (4:1) solution. The OD₅₇₀ was determined from this solution, and the biofilm volume was calculated as the ratio of OD₅₇₀ to OD₆₀₀.

Biofilm Inhibition Assays: For biofilm formation on polystyrene surfaces, flat-bottom 96-well microtiter plates (Corning Inc.) were used. The effect of different compounds on biofilm formation was tested by adding compounds at different concentrations to the bacterial culture (100× diluted overnight culture; approximately 10⁷ cells) in fresh LB medium. Two hundred microliter aliquots were pipetted into 96-well plates and placed for 24 or 48 h into a 37° C. incubator. The biofilm mass was measured by the CV staining assay described elsewhere herein.

Biofilm Persistence Assays with Ampicillin: Biofilms were grown for 24 or 48 h as described elsewhere herein. Planktonic cells were removed, and the biofilms were washed twice with 250 μL sterile PBS solution. Two hundred microliters of fresh LB medium with various concentrations of ampicillin were dispensed into the wells. After 18 h of incubation at 37° C., the volume of biofilm was measured by CV staining as described elsewhere herein.

Synergistic Effects of Compounds and Antibiotics: Biofilms were grown for 24, 48, or 72 h as described elsewhere herein. Planktonic cells were then removed, and biofilms were washed twice with 250 μL sterile PBS solution. Two hundred microliter aliquots of fresh LB medium with multiple concentrations of the tested compounds and ampicillin were dispensed into the wells. After 18 h of incubation at 37° C., the biofilm mass was measured as described elsewhere herein. For the AlamarBlue viability test, 4 μL of AlamarBlue (Invitrogen) was added and plates were incubated in a Biotek HT plate reader at 37° C. for 4 h. Cell viability was measured as fluorescence at 530/590 nm (excitation/emission) versus compound concentration or initial cell density.

Cell Growth Curves: The effect of the hit compounds on bacterial growth was tested by adding compounds at multiple concentrations to the bacterial culture (100× diluted overnight culture; approximately 10⁷ cells) in fresh LB medium. Two hundred microliters aliquots were pipetted into 96-well plates and placed into a Biotek HT or Tecan Infinite M200 Pro plate reader for 18 h at 37° C. Plates were shaken during incubation and the optical density (OD₆₃₀ or OD₆₀₀) was measured every 15 min.

Antibiotic Susceptibility Assays: For liquid cultures, the minimal inhibitory concentrations (MICs) of the antimicrobial drugs were determined using 96-well plates and the broth dilution method. Suspensions were then incubated at 37° C. for 18 h in a Biotek HT plate reader (see bacterial growth). Biofilm destruction experiments were performed with different antibiotic concentrations, and cell densities were measured after 18 h. Bacterial concentrations were calculated via optical density (OD₆₃₀), and the lowest concentration causing 80% growth inhibition relative to the growth of the control was deemed to be the MIC.

Scanning Electron Microscopy (SEM) of Biofilm: E. coli biofilms were grown in LB with multiple concentrations of the hit compounds on metal pins. These metal pins were then washed twice in 1×PBS. The biofilm-containing metal pins were then placed in a 5% glutaraldehyde solution for 1 h. Metal pins were then dried using a gradient of ethanol from 50% to 100%, 5 min in each solution. The pins were sputter coated with gold at a thickness of 60 Å. SEM images were taken on a Zeiss Supra 50VP Scanning Electron Microscope 5 kV beam acceleration.

Statistical Analysis: Statistical analyses were performed using OriginPro 8.5. Relevant statistical data is included in results and discussion for each experiment. Error bars indicated standard deviation from the mean. Asterisks represent statistical significance of at least p<0.05.

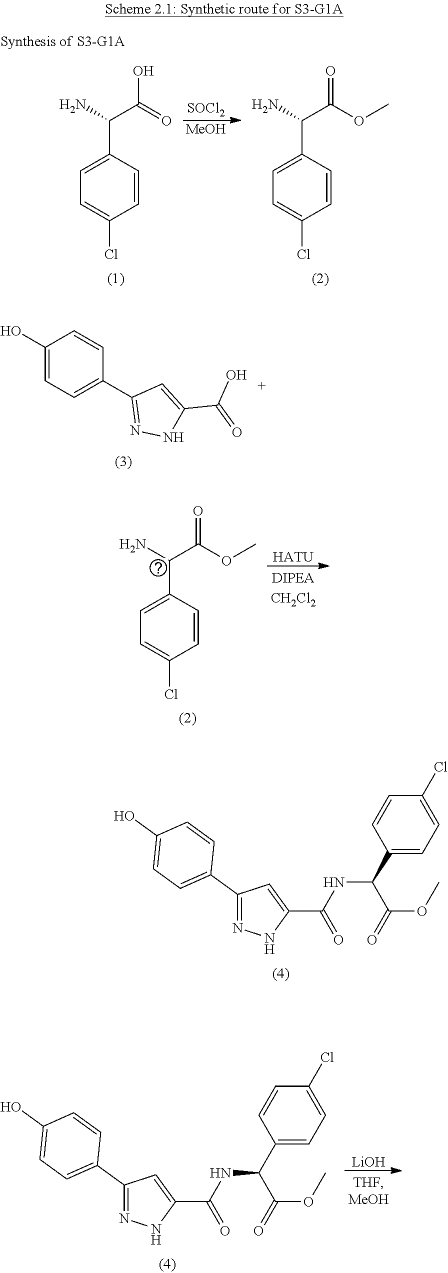

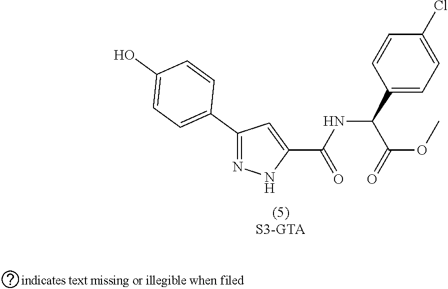 [SMILES: COC(=O)[C@@H](NC(=O)c1cc(-c2ccc(O)cc2)n[nH]1)c1ccc(Cl)cc1]

Methyl (S)-2-amino-2-(4-chlorophenyl) acetate (2). 0.5 g (2.69 mmol) of (S)-2-amino-2-(4-chlorophenyl) acetic acid (Ark Pharm) (1) was charged into a round bottom flask. 8 mL of methanol was added to the flask and cooled to 0° C. Thionyl chloride (0.819 g, 6.89 mmol) was added to the flask dropwise over a period of 5 mins. Mixture was allowed to come to room temperature and then subsequently heated to reflux for 3 h. Mixture was cooled and then concentrated in vacuo to afford a white-pink solid as a salt. Yield=0.61 g, 96.02%. 1H NMR (500 MHz, d6-DMSO) δ (PPM) 9.14 (S, 3H), 7.54 (dd, J=3.2, 1.4 Hz, 4H), 5.34 (d, 3.2 Hz, 1H), 3.70 (dd, J=3.1, 1.4 Hz, 3H). 13C NMR (500 MHz, d6-DMSO) δ (PPM) 130.73, 132.00, 129.47, 54.99, 53.74.

Methyl (S)-2-(4-chlorophenyl)-2-(3-(4-hydroxypehnyl)-1H-pyrazole-5-carboxamido) acetate (4). 0.3 g of methyl (S)-2-amino-2-(4-chlorophenyl) acetate chloride salt (3), 0.26 g of 3-(4-hydroxyphenyl)-1H-pyrazole-5-carboxylic acid (2) (ChemBridge), and 0.48 g of HATU (P3bioSystems) were added to RBF. This was followed by addition of 5 mL of dichloromethane and 0.26 g of DIPEA. The mixture was stirred at rt for 24 h. Mixture was concentrated in vacuo. Resulting oil was dissolved in ethyl acetate and washed with saturated solutions of ammonium chloride, sodium bicarbonate, and brine. This was followed by a water wash. Ethyl acetate was then dried with sodium sulfate and concentrated in vacuo affording a yellow-orange oil which solidified over overnight at 4° C. Product was taken to next step without purification. Yield=220 mg, 44%. 1H NMR (500 MHz, d6-DMSO) δ (PPM) 13.49 (s, 1H), 9.72 (s, 1H), 8.67 (s, 1H) 7.58 (t, J=8.7 Hz, 1H), 7.47 (m, 3H), 7.39 (m, 2H), 6.83 (m, 1H), 5.69 (d, J=7.4 Hz, 1H), 4.58 (s, 1H), 3.67 (d, J=4.1 Hz, 2H). 13C NMR (500 MHz, d6-DMSO) δ (PPM) 171.02, 130.40, 129.33, 129.26, 116.13, 52.99, 52.43, 38.68. HRMS m/z: [(M+H)+] calcd for C19H17O4N3Cl 386.09021, found 386.09102.

(S)-2-(4-chrlorphenyl)-2-(3-(4-hydroxyphenyl)-1H-pyrazole-5carboxamido) acetic acid (5). Crude product (4) was used in this reaction. (4) 220 mg was dissolved in 8 mL of THF/Water 1:1. 0.12 g of LiOH was then added. Reaction was allowed to stir at rt for 12 h. THF was then evaporated off in vacuo and aqueous solution was washed with ethyl acetate. Aqueous mixture was then washed with methylene chloride. Resulting aqueous mixture was then acidified with 1 M HCl resulting in precipitated product. Product was dried in vacuo overnight. Product was white powder. Yield=190 mg, 89%. 1H NMR (500 MHz, d6-DMSO) δ (PPM) 9.73 (S, 1H), 8.41 (S, 1H), 7.59 (m, 3H), 7.45 (m, 4H), 6.82 (m, 2H), 6.98 (S, 1H), 5.47 (S, 1H). HRMS m/z: [(M+H)+] calcd for C18H1504N3Cl 372.07456, found 372.07491.

### Example 3: Optimized Pipeline—Hit Compounds C14 and C22

The initial search for RelA inhibitors identified S3-G1A and S3-G1B as initial hit compounds; while they were able to inhibit RelA at relatively high concentrations, they were not viewed as ideal to take to structure-activity relationship studies. In Example 3, the work on the discovery of two new additional hit compounds for the inhibition of the RelA enzyme is described. This work builds upon the work described in Example 2 with the development of protocols for the RelA drug discovery pipeline. This work also incorporated the use of new computational tools to streamline the in silico docking models. Compound C14, determined to be the most active compound, was taken to in vitro toxicity models and early animal models using Galleria mallonella. In this section, a finding of the ability of C14 to essentially eliminate pyocyanin production in Pseudomonas aeruginosa is described.

### Example 3-1: Discovery of C14 and C22

### Expanded Docking Libraries

One of the most important parts of in silico docking studies is the large libraries that need to be run to determine hit compounds. In Example 2, the use of the Zinc database allowed docking for a database of around 4 million ligands, not all purchasable at the time of docking. A new library, provided by Enamine® (a company that supplies hit-to-lead drug design services), has been run using an updated docking strategy from Example 2. Enamine®'s high-throughput screening (HTS) library contains over 2 million unique compounds, all of which are purchasable in quantities of 1-10 mg. This service provides the ability to run docking studies, followed by purchasing of hit compounds for lab studies. Enamine® also provides similarity searches for analogs in their available stock. Once an in silico hit compound is has been confirmed using bioassays, a similarity search can be conducted providing ligands that are analogs to the initial compound of interest. This allows for rapid structure-activity relationship studies to be conducted without the need for synthesizing analogs. These structure searches are beneficial to provide rapid determination of bioisosteres.

### Optimized in Silico Docking Models

Initially, docking models were completed in a step-by-step manual system. Each stage of the docking model was completed by selecting the input ligands from a set of output ligands and there was no automation to this process. This method was time consuming and added a few days to the docking models. With the advancement of the docking protocols came the use of KNIME®. This open-source pipeline management program allows for the automation of Schrödinger® docking software such as Glide and LigPrep.

The pipeline shown in FIG. 26 is an example of a start-to-finish docking model. To run this model, one only needs to input a target grid file and a file of ligands. To start, KNIME® will call on LigPrep and prepare the ligands according to input parameters. Once completed, ligand files will be generated in two formats (SDF, Maestro). The Maestro file will be funneled into a Glide docking simulation, where it will meet a grid file, and the high-throughput virtual screen (HTVS) docking simulation will begin. The output of the docking will be saved, and the top 15% docking scores are then funneled into the standard precision (SP) Glide docking simulation. From the SP, the top 10% scoring ligands will be saved and input into the extra precision (XP) docking model. The output will also be written, and both the Maestro and SDF files, as well as the ligands, can be evaluated. This system allows for a nonstop docking simulation to run from start to finish and drastically cuts down on the simulation time.

### Improvement of Grid File

In Example 2, a homology model of RelA and RelP revealed a better understanding of the docking of GTP and ATP, which allowed for mutation studies of key amino acid residues to show loss of function. A utilization of this information in in silico models was then introduced for the determination of future hit compounds. Amino acid Y314 was found to be essential to the binding of the amine base of GTP. A grid structure was then constructed using Y314 as the center of this docking grid, allowing for a slightly more accurate docking target.

### Semi-HTS of New in Silico Hits

With the establishment of in vitro RelA enzymatic assays in Example 1, a semi-HTS can be accomplished. This approach allows for a larger set of ligands to be tested from the in silico docking simulations. After the docking of the Enamine® library, 40 compounds (FIG. 27) were determined to have binding scores above the threshold of −9.5 kcal/mol.

The next step of the semi-HTS was to run the 40 in silico determined compounds in an in vitro RelA assay at a relatively high concentration of 200 μM, to test if there is any effect on RelA ppGpp production using the α-32P-GTP radio isotope in vitro assay. FIG. 28 shows ppGpp production relative to the untreated control; this untreated control is set at 100% and all compounds are scaled according to this control. From this assay, two compounds were picked to go into later assays (C14 [4.2%], C22 [13.4%]). The graph in FIG. 28 shows several of the compounds act as an agonist and increase the production of ppGpp compared to the untreated control.

### IC₅₀ of Selected Molecules

The selected molecules from the semi-HTS were chosen because both showed percentages lower than the 15% of the untreated control RelA; these compounds can be seen in FIG. 29A. The follow-up assay to the semi-HTS is determination of in vitro half-maximal inhibitory concentration (IC₅₀). In vitro IC₅₀ is completed by a concentration gradient run on the in vitro radio isotope assay (FIG. 29B). The in vitro IC₅₀ for C14 and C22 are 54 μM and 93 μM, respectively. The C14 IC₅₀ of 54 μM is in the range of a compound that can be optimized for higher binding through structure activity relationship (SAR) studies. C14 was originally purchased from Enamine®; however, it was later synthesized in-house as more was needed than could be purchased.

### Example 3-2: Biological Assays of New Hit Compounds

### Compound Effect on Bacteria Growth

Because compounds themselves should not be toxic to the bacteria, the first biological assay is a growth curve analysis. For this model, the present study chose to determine if growth of Escherichia co/i C and Pseudomonas aeruginosa (PA01) was affected by the chosen compounds. P. aeruginosa a gram-negative pathogen that commonly causes chronic wound and lung infections. In Example 2, S3-G1A and S3-G1B showed no effect on bacterial growth and ΔrelA mutants grew in a manner similar to that of the wild-type bacteria. As shown in FIGS. 30A-30B, the 18-h growth is not affected by the compounds at concentrations as high as 200 μM.

E. coli C Aggregation Assay

Previously determined by Krol et al., E. coli C, under certain stress conditions, aggregates together and forms biofilms from a planktonic culture. This process is proposed to work under a CsrA stress response. In the case of E. coli C, the level of CsrA expression is lower due to the presence of an insertion sequence in its promoter. Because RelA is essential in the production of (p)ppGpp in inducing CsrA stress response, it was hypothesized that inhibition of RelA would limit aggregation of E. coli C. It was found that C14 and C22 reduced the amount of E. coli C aggregation under high salt and low temperature and amino acid starvation (FIGS. 31-33). This aggregation process is proposed to be controlled by the CsrA/NhaR mechanism through overproduction of polysaccharide adhesin poly-β-1,6-N-acetyl-d-glucosamine (FIG. 31). However, more studies need to be performed to confirm RelA and (p)ppGpp involvement in the aggregation effect, including in the ΔrelA E. coli C mutant.

Compound Effect on Biofilm Formation in P. aeruginosa PA14

As noted in Example 2, compounds have little effect on biofilm formation, and this was also observed for C14 and C22 with E. coli C and P. aeruginosa PA01 (results not shown). However, when P. aeruginosa PA14 was treated with the compounds, a decrease in biofilm was noted. PA14 is a strain of P. aeruginosa that is particularly virulent and displays many quorum sensing and virulence factors such as biofilm formation. C14 significantly decreased biofilm formation compared to the DMSO control (P=0.0035) (FIG. 34). C22 also reduced biofilm formation but not significantly.

Compound Effect on Pyocyanin Production in P. aeruginosa A serendipitous effect observed from the treatment of P. aeruginosa PA14 with Compounds C14 and C22 was their effect on pyocyanin production. While a small effect was noted with treatment of S3-G1A and S3-GIB, when PA14 was treated with C14, production of pyocyanin was almost completely shut down. Below 625 nM, the effect was still observed (FIG. 35B). While C22 produced an effect, it never reached the level of C14 (FIG. 35A). Several previous works have shown that pyocyanin production is correlated with the (p)ppGpp levels in the cells. In these works, the RelA/SpoT Homolog (RSH) mutant strains produced fewer virulence factors such as pyocyanin. Much work still needs to be done on this front, and a better picture of RelA's role in pyocyanin production needs to be derived.

Galleria mallonella (Wax Moth) Model

The Galleria mallonella killing models have been used now in numerous ways in the development of antimicrobial agents. These models can evaluate early in vitro toxicity of potential drugs, evaluate the efficacy of an agent in respect to its antimicrobial function, and work as a model for bacterial and fungal pathogenicity. This model consists of injection of roughly 5 μL of the solution of interest (bacteria, compounds, combinations) into the back leg of the larvae. These models act as a preliminary stand-in for the early stages of murine models. Murine models have monetary and ethical issues associated with starting an early model system. The G. mallonella model is cheap and simple to work with.

One aspect that makes the G. mallonella model simple to utilize is the interesting characteristic of the G. mallonella larvae of changing color when dead. This is due to the immune response in many insects where there is a melanization of hemolymph through multiple enzymatic cascades. The coagulation of hemocytes around the infection adds to the full color change of the larva. This immune response and subsequent melanization result in the G. mallonella turning from white to brown and then finally black, when death has occurred. There are different scales to rank the melanization process and the immune response noted in the larvae. This scale ranks the G. mallonella as either white (not sick), orange/brown (sick), and black (dead if there is no movement).

Because C14 was found to be the most effective compound in all in vitro and microbiological assays, it was decided to test C14 toxicity in the G. mallonella model. This was carried out by the injection of C14 diluted in sterile phosphate-buffered saline (PBS) into the back left proleg of the larva. The reason for this injection site is to ensure consistency within the model at all times. Before the larvae are utilized in the assay, they are checked for size and sickness (melanization). There was no observed effect of a 5-μL injection of 50 μM C14 after 48 h (FIGS. 36A-36B).

To test for the ability of C14 to reduce the pathogenicity of PA14, PA14 overnight culture was resuspended in PBS and then supplemented with 50 μM C14 or DMSO such that a 5-μL injection contained roughly 5×10⁵ cells. C14 decreased the death of the G. mallonella (FIGS. 37A-37E): at 48 h, 95% of the larvae treated with C14 survived, whereas all the larvae treated with DMSO control died.

G. mallonella PA14 Toxin Killing

To show that C14 reduces the toxins produced by P. aeruginosa, cell cultures were treated with C14 and grown as described previously. C14 reduced the amount of pyocyanin produced by the PA14 strain compared to the DMSO control. To show this reduction of toxin transferred into a relative model, the supernatant of the overnight cultures was collected and sterilized by filtration. This sterilized supernatant was then injected into the G. mallonella larvae. C14 resulted in a decreased killing in the G. mallonella larvae. After 84 h, 40% of the C14-treated larvae were still alive while 100% of larvae treated with the DMSO control were killed (full killing curve in FIG. 38), demonstrating the reduced virulence produced by C14 treatment.

### Lactate Dehydrogenase Toxicity Assay of C14

As a toxicity assay with human cells, the lactate dehydrogenase (LDH) colorimetric cytotoxicity assay was performed using Compound C14. This assay works by measuring the cell membrane integrity. When a cell dies, either by necrosis or apoptosis, the cell membrane becomes compromised. This allows many enzymes to leak into the extracellular space. One of the leaked enzymes is LDH, a stable enzyme that can be utilized as an indicator of cell death. The LDH assay works by a conversion of iodonitrotetrazolium into formazan (red-colored compound). LDH oxidizes lactate to pyruvate by means of the reduction of nicotinamide adenine dinucleotide (NAD+) to NADH. Another enzyme diaphorase is then able to oxidize NADH to NAD+ by reducing and transferring a proton to indonitrotetrazolium to form formazan (FIG. 39B). This color change can then be compared to a standard and a cytotoxicity percentage can be reported.

When cells were allowed to grow for 24 h in the presence of C14, there was no noted cytotoxicity (FIG. 39A) compared to that of the water (w/DSMO) control. The cells utilized for this assay were A549, which is a human alveolar basal epithelial cell line harvested from a 58-year-old male in 1972. These cells form a confluent monolayer on the culture flask and are often used for simple LDH cytotoxicity assays.

### Example 3-3: C14 SAR Studies

### Initial SAR Studies

The use of Enamine® as a source for hit compounds has another advantage over other ligand databases. Enamine® stores analogs to most of the compounds that can be ordered; that is to say, a similarity search can be performed on the hit structure and compounds with similar moieties can be purchased for quick SAR analysis. A C14 search on Enamine®, at 90% similarity, gave several compounds that were able to be tested in vitro for their efficacy. The loss of the carboxylic acid on C14 was found to result in loss of RelA inhibition efficacy in the in vitro assays. Each compound was tested at 200 μM, and none of them had any effect on ppGpp production. The similarity search yielded compounds that had exchanged the carboxylic acid for an alcohol, methyl ester, and amide (FIG. 40).

Optimization of C14 with Molecular Docking for Future Synthesis

To continue the SAR studies of C14 for lead compound optimization, a program called Cresset™ Spark™ was utilized. This program works to generate possible bioisosteres exchanges that can be put into an existing docking model, and if the docking score is in the acceptable range, a synthesis route can be established, and a compound synthesized. Spark™ works by creating an electrostatic and space map of the protein's docking site. This allows for R-groups to be exchanged from a large set of potential R-groups. This process is also scored on the final molecule's adherence to a set of “drug-like” rules such as Lipinski's rules. This process allows for full exploration of the chemical space while keeping the initial hit compound relevant. Hit compound C14 was optimized by splitting it into 3 sections and running the R-group exchange on each section (FIG. 41A). This optimization led to the compounds in FIG. 41B. These compounds were then run back through the docking model with RelA. These compounds are synthesized and tested through the now developed biological pipeline.

### Example 3-4: Experimental

### Enamine® HTS in Silico Docking Studies

The RelA enzyme (PDB: 5IQR) was prepared and optimized using Maestro Protein Preparation (Schrödinger, LLC, New York, NY, USA; Version 11.9.011, MMshare Version 4.5.011, Release 2019-1, Platform Windows-x64). The dockable RelA structure was prepared and minimized using Schrödinger® protein preparation application.23 This application was utilized to add hydrogens, create missing disulfide bonds, and determine lowest-energy residue orientations. Geometry minimization was carried out using the force field OPLS3e.24 A docking site was determined using homology studies of bacterial rel genes from several species in combination with the Schrödinger® binding site determination tool. Enamine® ligands were prepared using Schrödinger® LigPrep (Schrödinger Release 2020-1: LigPrep, Schrödinger, LLC, New York, NY, USA, 2020).

Semi-HTS of Compounds and In Vitro (p)ppGpp Quantification

In vitro (p)ppGpp quantification was carried out. RelA enzyme was purified as described in Example 2. Roughly 0.4 μg of RelA protein was added to a 1.5-mL microcentrifuge tube containing a reaction mix composed of 1×PBS, 5 mM MgCl₂, 0.5 mM ATP, 0.5 mM GTP, 0.5 mM GDP, and 20 μCi [α-32P]GTP (3,000 Ci mmol-1; PerkinElmer) and 200 μM of each of the 40 compounds was added to the mixture. These reactions were incubated at 37° C. for 1 h. The reactions were stopped by addition of 5 μL formic acid (88%). The reaction mixtures were then spotted on a stationary-phase polyethyleneimine (PEI)-cellulose TLC plate using potassium phosphate monobasic (1.5 M) as the mobile phase. The plates were then dried, and the radiation levels were read using a Molecular Dynamics Storage Phosphor Screen. A Molecular Dynamics Storm 840 Phosphor Imager Scanner was used to read the phosphor screen, and ImageJ was used to process the images.

### Bacterial Growth Curves

The effect of the hit compounds on bacterial growth was tested by adding compounds at 200 μM (with DMSO control) to the bacterial culture (100× diluted overnight culture; approximately 10⁷ cells) in fresh LB medium. Two hundred microliter aliquots were pipetted into 96-well plates, which were placed into a Biotek HT or Tecan Infinite M200 Pro plate reader for 18 h at 37° C. Plates were shaken during incubation and the optical density (OD₆₃₀ or OD₆₀₀) was measured every 15 min.

### Biofilm Aggregation Assay

E. coli C was grown in LB Miller broth at 30° C. with shaking (250 rpm) with or without compounds. E. coli C ΔrelA mutant was also grown as a control. One milliliter of the culture was transferred to standard polypropylene spectrophotometer cuvettes to measure planktonic cells densities (OD₆₀₀). Remaining cultures were vortexed ˜1 min and 1 mL was aliquoted into cuvettes to measure the total cell densities (OD₆₀₀). Aggregation was calculated as a ratio of planktonic to total cell density.

### Pyocyanin Production Assay

P. aeruginosa PA14 was grown overnight in LB Miller broth at 37° C. with shaking (250 rpm). Five milliliters of LB miller broth was supplemented with Compounds C14 and C22 at various concentrations and inoculated with 10 μL of overnight PA14. This was then grown for 18 h at 37° C. Cells were pelleted by centrifugation. One milliliter of supernatant was collected, and 1 mL of chloroform was added to the supernatant. Layers were separated and OD₆₉₀ was taken for chloroform layer (blue). OD₆₉₀ was normalized to culture cell density OD₆₀₀.

G. mallonella Model

C14 Toxicity Assay. G. mallonella were purchased from Amazon. C14 was diluted to 50 μM in PBS. G. mallonella larvae were injected in the back left proleg with 5 μL of C14 solution and DMSO control using a syringe pump and 1-mL insulin syringe. G. mallonella larvae were then counted live/dead twice daily. Twenty-five G. mallonella larvae were utilized in triplicate for each experiment.

P. aeruginosaPA14 Infection Model. Bacterial pellets were diluted in C14 and DMSO-supplemented PBS so that 5-μL injections contained roughly 1×10⁵ cells. Five microliters of the PBS solution was injected into the back left proleg of the G. mallonella larvae using a syringe pump and 1-mL insulin syringe. G. mallonella larvae were then counted live/dead twice daily. Twenty-five G. mallonella larvae were utilized in triplicate for each experiment.

G. mallonella PA14 Toxin Killing. P. aeruginosa PA14 was grown overnight in LB Miller broth at 37° C. with shaking (250 rpm). Five milliliters of LB Miller broth was treated with Compound C14 at 50 μM. Ten microliters of overnight PA14 was then inoculated into the 5 mL of treated LB. This was then grown for 18 h at 37° C. Cells were pelleted by centrifugation, and 1.5 mL of supernatant was collected and filter-sterilized using a 0.2-μm syringe filter (VWR). Five microliters of resulting sterilized supernatant was injected in the back left proleg of the G. mallonella larvae. G. mallonella larvae were then counted live/dead twice daily. Twenty-five G. mallonella larvae were utilized in triplicate for each experiment.

### LDH Cytotoxicity Assay

A549 cells were seeded at 9,000 cells per well in 200 μL Dulbecco's modified Eagle's medium supplemented with 10% (v/v) fetal bovine serum, 2 mM L-glutamine, and penicillin-streptomycin (100 IU, 0.1 mg/mL). Cultured cells were treated with various concentrations of C14 and incubated for 24 h at 37° C. in 5% CO₂. LDH activity was examined using a Thermo Scientific™ Pierce™ LDH Cytotoxicity Assay Kit following the manufacturer's instructions. These measurements were performed on a Tecan Infinite M200 Pro plate reader.

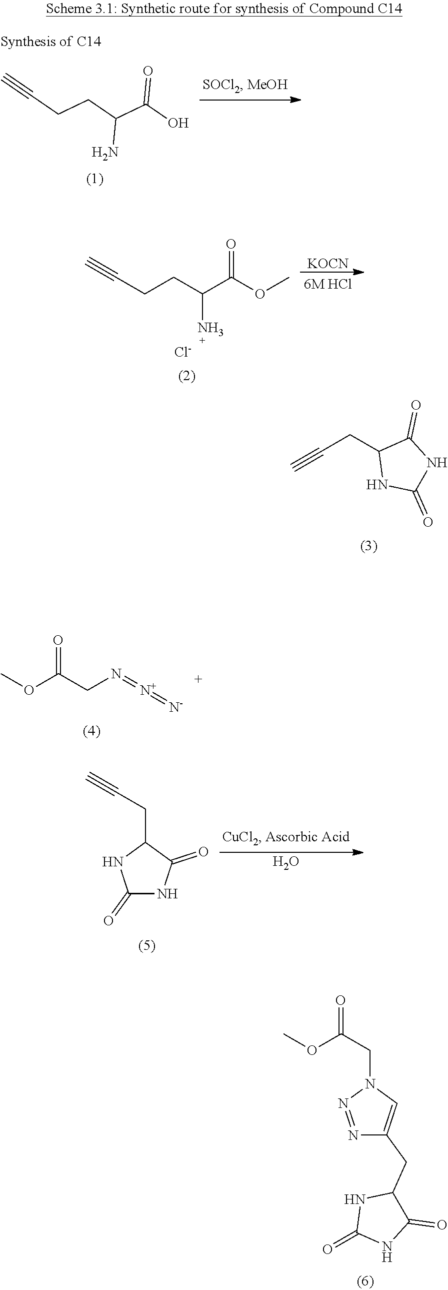

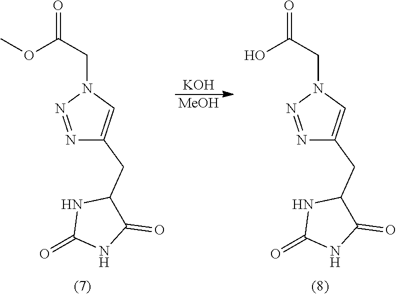 [SMILES: "MeOH" ; COC(=O)Cn1cc(CC2NC(=O)NC2=O)nn1 ; "KOH" ; O=C(O)Cn1cc(CC2NC(=O)NC2=O)nn1]

1-methoxy-1-oxohex-5-yn-2-aminium chloride (2). 1.78 mmol of 2-aminohex-5-ynoic acid (1) was charged in an RBF with 10 mL of dry methanol. This mixture was cooled to 0° C. in an ice bath. 11.3 mmol of thionyl chloride was added dropwise over a period of 10 min to the cooled mixture. The mixture was allowed to warm to room temperature followed by reflux for 3 h. It was then cooled and concentrated in vacuo to afford a yellow oil. This oil was washed with diethyl ether followed by high vacuum overnight. Quantitative yield. 1H NMR (500 MHz, d6-DMSO) δ (PPM) 8.84 (s, 3H), 4.19 (d, J=5.7 Hz, 1H), 3.54 (s, 3H), 3.12 (d, J=5.0 Hz, 1H), 2.83 (dd, J=6.1, 3.0 Hz, 2H). 13C NMR (500 MHz, d6-DMSO) δ (PPM) 168.87, 77.73, 53.40, 51.11, 20.43.

5-(prop-2-yn-1-yl) imidazolidine-2,4-dione (3). 1.8 mmol of (2) was added to the RBF with 2.4 mmol KOCN and 10 mL of water followed by a reflux for 2 h. The mixture was allowed to cool, and 3.3 mL of H2SO₄ (conc.) was added dropwise. The mixture was allowed to reflux for 2 h. It was then was cooled to room temperature and 15 mL of brine was added to mixture. This mixture was extracted with 500 mL of ethyl acetate. Ethyl acetate was concentrated in vacuo resulting in a yellow oil, which was placed at 4° C. overnight. The oil solidified into a yellow solid. Yield: 184 mg, 74%. 1H NMR (500 MHz, d6-DMSO) δ (PPM) 13.80 (s, 1H), 10.09 (s, 1H), 4.71 (t, J=4.7 Hz, 1H), 3.22 (m, 2H) 2.90 (t, J=2.7 Hz, 1H). 13C NMR (500 MHz, d6-DMSO) δ (PPM) 157.93, 73.92, 56.41, 21.46. HRMS m/z: [(M+H)+] calcd. for C6H7N2O2 139.05020, found 139.05029.

methyl 2-(4-((2,5-dioxoimidazolidin-4-yl) methyl)-1H-1,2,3-triazol-1-yl)acetate (6). 0.72 mmol of (3), 0.11 mmol copper sulfate, and 0.32 mmol of sodium ascorbate were added to the RBF with 15 mL of 1:1 THF:water and stirred for 10 min. 1.3 mmol of methyl 2-azidoacetate (4) was added dropwise. The mixture was allowed to stir at room temperature for 7 h. It was then concentrated to half its volume in vacuo. It was extracted with 400 mL of ethyl acetate, dried with sodium sulfate, and concentrated in vacuo. The final product was a white solid. Yield: 88.4 mg, 48%. 1H NMR (500 MHz, d6-DMSO) δ (PPM) 10.54 (s, 1H), 7.87 (d, J=15.5 Hz, 2H), 5.36 (s, 2H), 4.35 (m, 1H), 3.69 (m, 3H), 3.03 (m, 2H). 13C NMR (500 MHz, d6-DMSO) δ (PPM) 175.56, 168.11, 157.69, 141.84, 125.05, 57.49, 52.90, 50.62, 27.60. HRMS m/z: [(M+H)+] calcd. for C9H12N5O4 254.08838, found 254.08827.

2-(4-((2,5-dioxoimidazolidin-4-yl) methyl)-1H-1,2,3-triazol-1-yl) acetic acid (8). 0.197 mmol (6) was added to the RBF with 5 mL of dry methanol followed by 0.394 mmol of KOH. This mixture was heated to 40° C. for 12 h. The mixture was concentrated in vacuo. Then 5 mL of water was added, and it was acidified with 2 M HCl until pH 4. Then 0.5 mL of brine was added. It was extracted with 350 mL of ethyl acetate, dried with sodium sulfate, and concentrated to a white powder in vacuo. Yield: 14 mg, 29%. 1H NMR (500 MHz, d6-DMSO) δ (PPM) 13.27 (s, 1H), 10.54 (s, 1H), 7.86 (m, 2H), 5.22 (s, 2H), 4.31 (td, J=5.4, 1.4 Hz, 1H), 3.02 (qd, J=15.2, 5.3 Hz, 2H). 13C NMR (500 MHz, d6-DMSO) δ (PPM) 175.59, 169.00, 157.70, 141.74, 124.97, 57.50, 50.83, 27.65. HRMS m/z: [(M+H)+] calcd. for C8H10N5O4 240.07273, found 240.07267.

### Example 4: C14 Inhibits P. aeruginosa Toxin Production at Low Concentrations

In Example 3, both C14 and C22 were shown to inhibit the production of the toxin pyocyanin by P. aeruginosa (see e.g., FIGS. 35A-35B, 36A-36B, 37A-37E, and 38). Specifically, in two invertebrate animal models of infection, C14 was demonstrated to be able to protect the infected animals from death, when the untreated animals succumbed to the infection.

In Example 4, C14 was demonstrated to be able to almost completely shut down toxin production of the key bacterial pathogen, Pseudomonas aeruginosa, at low nanomolar concentrations. Referring to FIG. 42, C14 inhibits the production of toxin production by P. aeruginosa with a EC50 as low as 39.5 nM.

### Enumerated Embodiments

In some aspects, the present disclosure is directed to the following non-limiting embodiments:

Embodiment 1: A method of treating, ameliorating and/or preventing biofilm formation by a bacterium, the method comprising contacting the bacterium with at least one compound selected from:
(a) a compound of Formula I:

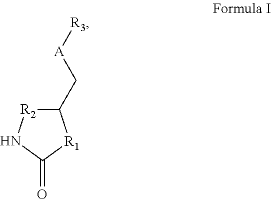

or a salt, solvate, tautomer, N-oxide, geometric isomer, stereoisomer thereof, and/or mixtures thereof, wherein:
   R₁ is —NH— or —O—,
   R₂ is —CH₂— or —C(O)—,
   A is a five member aromatic heterocyclic ring or —CH═CH—COO—*, wherein * is the bond to R₃.
   R₃ is —O—C(O)OH or

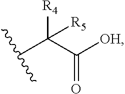

and
R₄ and R₅ are each independently H, halogen, C₁-C₆ alkyl, C₁-C₆ alkoxy, or —OH;
(b)

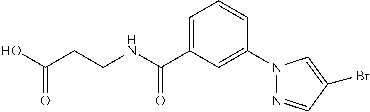 [SMILES: O=C(O)CCNC(=O)c1cccc(-n2cc(Br)cn2)c1]

3-(3-(4-bromo-1H-pyrazol-1-yl)benzamido)propanoic acid (C22), or a salt, solvate, tautomer, N-oxide, geometric isomer, and/or mixtures thereof.

Embodiment 2: The method of Embodiment 1, wherein in Formula I, A is

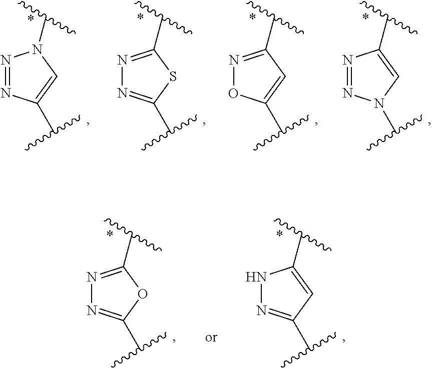 [SMILES: *c1cn(*)nn1 ; *c1nnc(*)s1 ; *c1cc(*)on1 ; *c1cn(*)nn1 ; *c1nnc(*)o1 ; *c1cc(*)[nH]n1]

wherein * is the bond to R₃ and wherein the CH in the five-membered heterocyclyl group of A (if present) is independently optionally substituted with at least one of C₁-C₆ alkyl, C₁-C₆ alkoxy, and halogen.

Embodiment 3: The method of any one of Embodiments 1-2, wherein the compound of Formula I is at least one selected from the group consisting of:

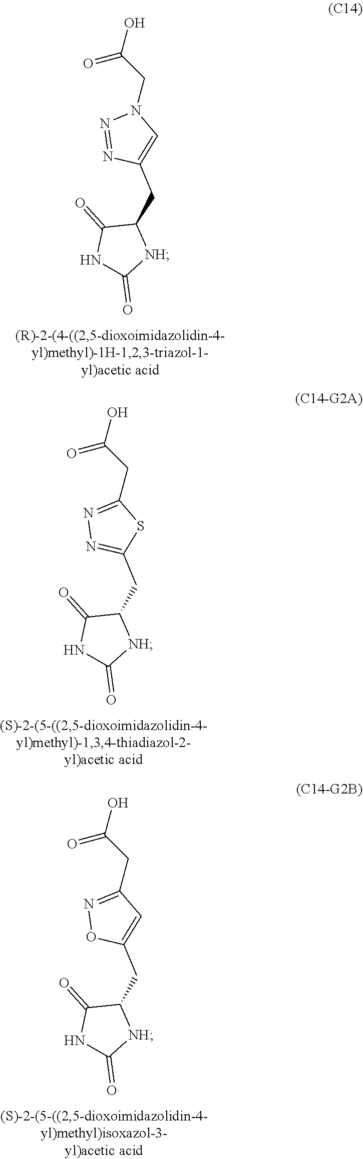 [SMILES: O=C(O)Cn1cc(C[C@H]2NC(=O)NC2=O)nn1 ; O=C(O)Cc1nnc(C[C@@H]2NC(=O)NC2=O)s1 ; O=C(O)Cc1cc(C[C@@H]2NC(=O)NC2=O)on1]

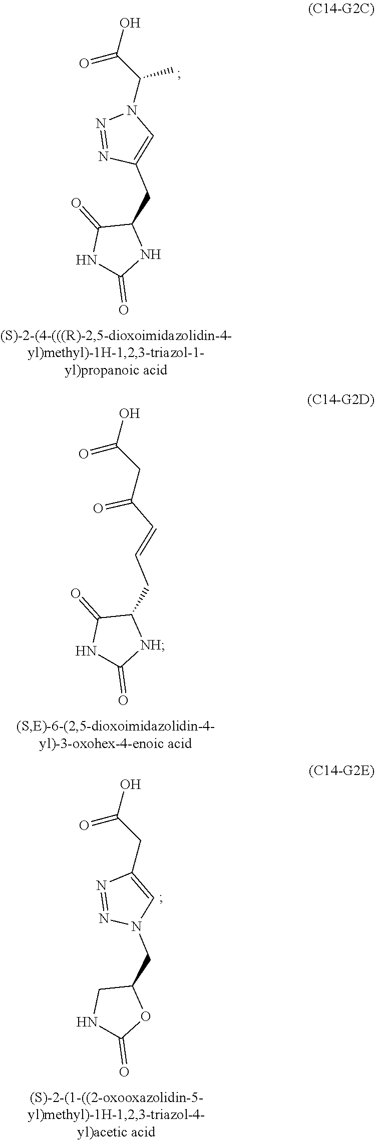 [SMILES: C[C@@H](C(=O)O)n1cc(C[C@H]2NC(=O)NC2=O)nn1 ; O=C(O)CC(=O)/C=C/C[C@@H]1NC(=O)NC1=O ; O=C(O)Cc1cn(C[C@@H]2CNC(=O)O2)nn1]

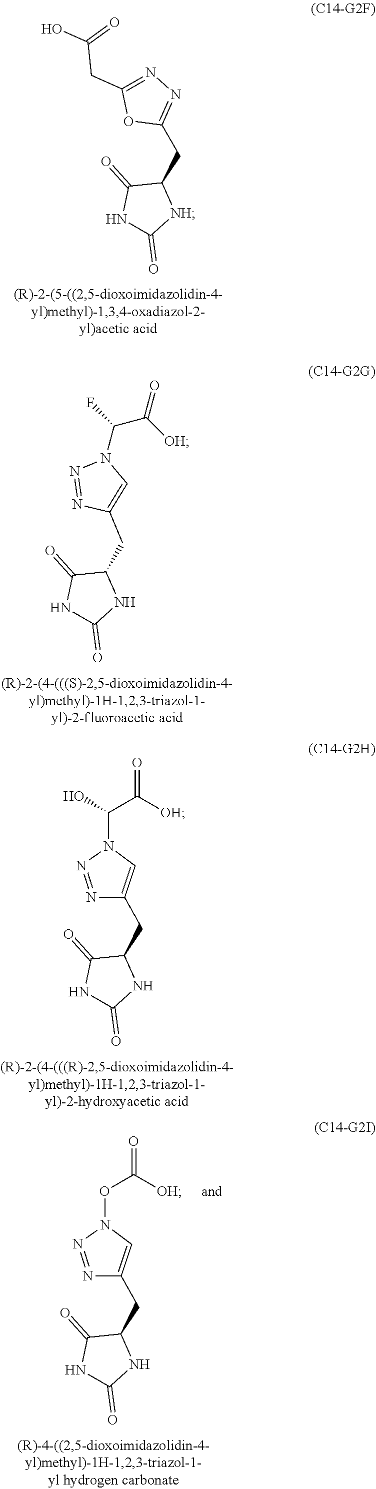 [SMILES: O=C(O)Cc1nnc(C[C@H]2NC(=O)NC2=O)o1 ; O=C1NC(=O)[C@H](Cc2cn([C@H](F)C(=O)O)nn2)N1 ; O=C1NC(=O)[C@@H](Cc2cn([C@H](O)C(=O)O)nn2)N1 ; O=C1NC(=O)[C@@H](Cc2cn(OC(=O)O)nn2)N1]

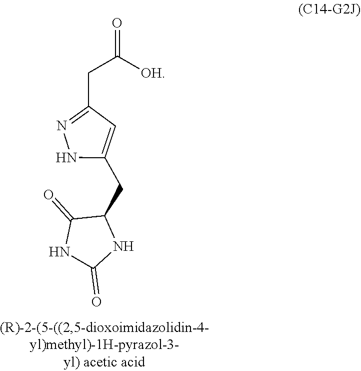 [SMILES: O=C(O)Cc1cc(C[C@H]2NC(=O)NC2=O)[nH]n1]

Embodiment 4: The method of any one of Embodiments 1-3, wherein formation of the biofilm by the bacterium is inhibited.

Embodiment 5: The method of any one of Embodiments 1-3, wherein the integrity of the bacterial biofilm already is compromised and/or reduced.

Embodiment 6: The method of any one of Embodiments 1-5, wherein the bacterium is a gram-positive bacterium or a gram-negative bacterium.

Embodiment 7: The method of any one of Embodiments 1-6, wherein the bacterium comprises a B. burgdorferi bacterium, an E. coli bacterium, an H. influenzae bacterium, an N. gonorrhoeae bacterium, a P. aeruginosa bacterium, an S. epidermidis bacterium, an S. pneumoniae bacterium, and/or an S. aureus bacterium.

Embodiment 8: The method of any one of Embodiments 1-7, further comprises contacting the bacterium with an antibiotic for killing and/or inhibiting the bacterium.

Embodiment 9: The method of any one of Embodiments 1-8, wherein the biofilm is present in and/or on a subject, and the method comprises administering and/or applying an effective amount of the at least one compound to the subject.

Embodiment 10: The method of Embodiment 9, wherein the biofilm is formed as part of a tissue-related infection in the subject or wherein the biofilm is formed in and/or on a device within the subject's body of the subject or in prolonged contact with the subject's body.

Embodiment 11: The method of any one of Embodiments 1-10, wherein the at least one compounds inhibits RelA or SpoT Homology (RSH) enzyme in the bacterium.

Embodiment 12: A method of inhibiting toxin production by a gram-negative bacterium, the method comprising contacting the gram-negative bacterium with at least one compound selected from:
(a) a compound of Formula I:

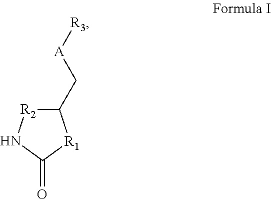

or a salt, solvate, tautomer, N-oxide, geometric isomer, stereoisomer thereof, and/or mixtures thereof, wherein:
   R₁ is —NH— or —O—,
   R₂ is —CH₂— or —C(O)—,
   A is a five member aromatic heterocyclic ring or —CH═CH—COO—*, wherein * is the bond to R₃.
   R₃ is —O—C(O)OH or

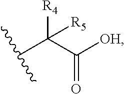

and
R₄ and R₅ are each independently H, halogen, C₁-C₆ alkyl, C₁-C₆ alkoxy, or —OH;
(b)

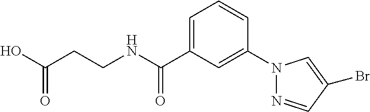 [SMILES: O=C(O)CCNC(=O)c1cccc(-n2cc(Br)cn2)c1]

3-(3-(4-bromo-1H-pyrazol-1-yl)benzamido)propanoic acid (C22), or a salt, solvate, tautomer, N-oxide, geometric isomer, and/or mixtures thereof.

Embodiment 13: The method of Embodiment 12, wherein in Formula I, A is

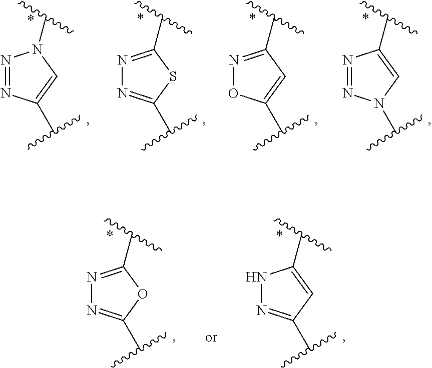 [SMILES: *c1cn(*)nn1 ; *c1nnc(*)s1 ; *c1cc(*)on1 ; *c1cn(*)nn1 ; *c1nnc(*)o1 ; *c1cc(*)[nH]n1]

wherein * is the bond to R₃ and wherein the CH in the five-membered heterocyclyl group of A (if present) is independently optionally substituted with at least one of C₁-C₆ alkyl, C₁-C₆ alkoxy, and halogen.

Embodiment 14: The method of any one of Embodiments 12-13, wherein the compound of Formula I is at least one selected from the group consisting of:

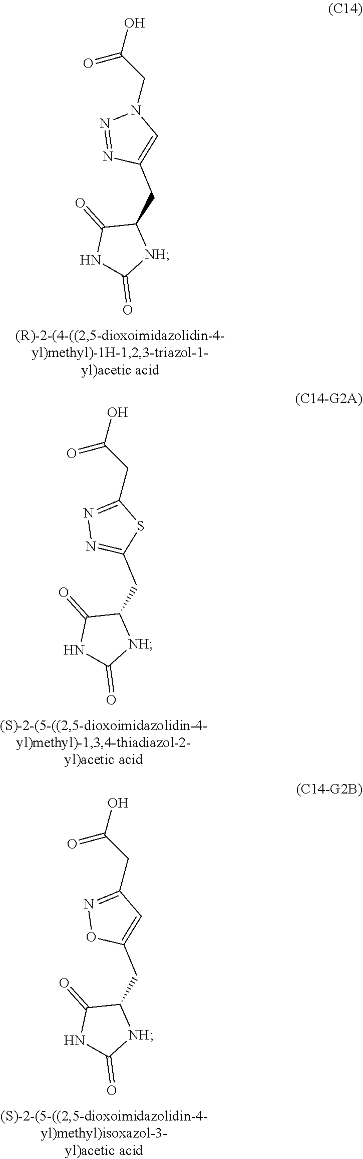 [SMILES: O=C(O)Cn1cc(C[C@H]2NC(=O)NC2=O)nn1 ; O=C(O)Cc1nnc(C[C@@H]2NC(=O)NC2=O)s1 ; O=C(O)Cc1cc(C[C@@H]2NC(=O)NC2=O)on1]

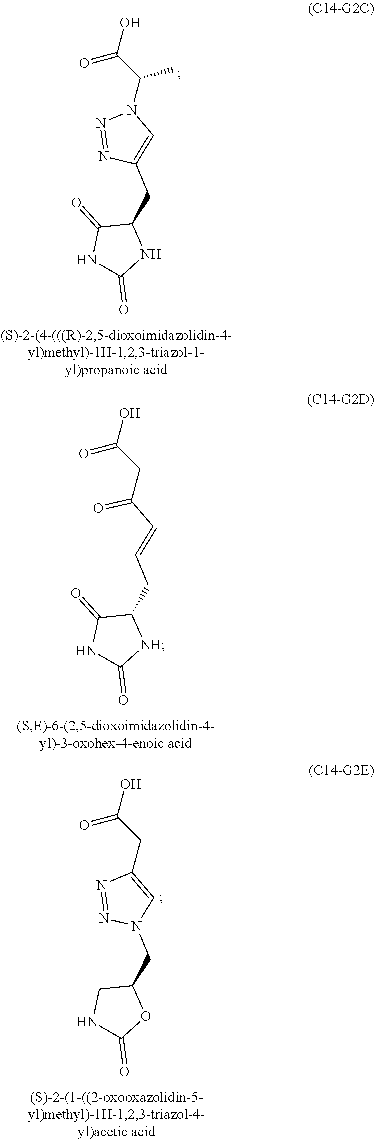 [SMILES: C[C@@H](C(=O)O)n1cc(C[C@H]2NC(=O)NC2=O)nn1 ; O=C(O)CC(=O)/C=C/C[C@@H]1NC(=O)NC1=O ; O=C(O)Cc1cn(C[C@@H]2CNC(=O)O2)nn1]

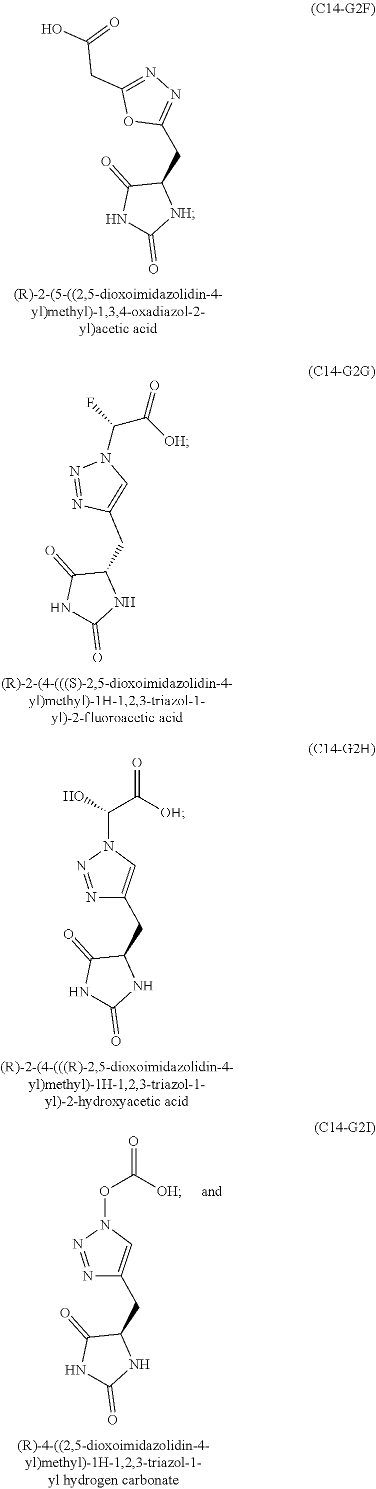 [SMILES: O=C(O)Cc1nnc(C[C@H]2NC(=O)NC2=O)o1 ; O=C1NC(=O)[C@H](Cc2cn([C@H](F)C(=O)O)nn2)N1 ; O=C1NC(=O)[C@@H](Cc2cn([C@H](O)C(=O)O)nn2)N1 ; O=C1NC(=O)[C@@H](Cc2cn(OC(=O)O)nn2)N1]

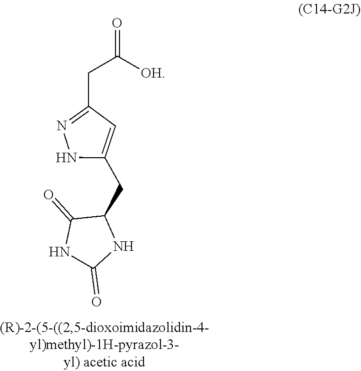 [SMILES: O=C(O)Cc1cc(C[C@H]2NC(=O)NC2=O)[nH]n1]

Embodiment 15: The method of any one of Embodiments 12-14, wherein the gram-negative bacterium comprises an E. coli bacterium, an H. influenzae bacterium, and/or a P. aeruginosa bacterium.

Embodiment 16: The method of any one of Embodiments 12-15, wherein the gram-negative bacterium is a cultured gram-negative bacterium.

Embodiment 17: The method of any one of Embodiments 12-15, wherein the gram-negative bacterium is in and/or on the body of a subject.

Embodiment 18: The method of Embodiment 17, wherein an effective amount of the at least one compound is administered and/or applied to the subject.

Embodiment 19: The method of any one of Embodiments 12-18, wherein RelA or RSH enzyme is inhibited in the gram-negative bacterium.

Embodiment 20: A compound represented by Formula I:

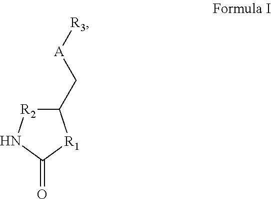

or a salt, solvate, tautomer, N-oxide, geometric isomer, stereoisomer thereof, and/or mixtures thereof, wherein:
   R₁ is —NH— or —O—,
   R₂ is —CH₂— or —C(O)—,
   A is a five member aromatic heterocyclic ring or —CH═CH—COO—*, wherein * is the bond to R₃.
   R₃ is —O—C(O)OH or

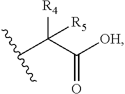

R₄ and R₅ are each independently H, halogen, C₁-C₆ alkyl, C₁-C₆ alkoxy, or —OH, and
the compound is not

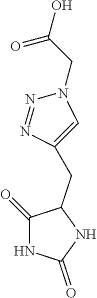 [SMILES: O=C(O)Cn1cc(CC2NC(=O)NC2=O)nn1]

2-(4-((2,5-dioxoimidazolidin-4-yl)methyl)-1H-1,2,3-triazol-1-yl)acetic acid (C14).

Embodiment 21: The compound of Embodiment 20, wherein in Formula I, A is

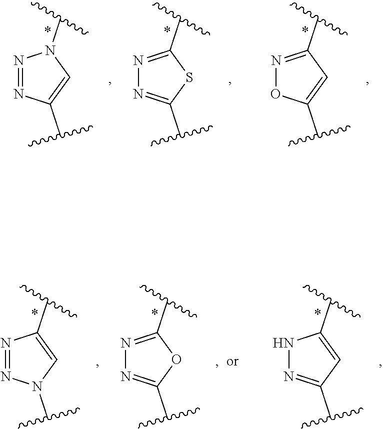 [SMILES: *c1cn(*)nn1 ; *c1nnc(*)s1 ; *c1cc(*)on1 ; *c1cn(*)nn1 ; *c1nnc(*)o1 ; *c1cc(*)[nH]n1]

wherein * is the bond to R₃ and wherein the CH in the five-membered heterocyclyl group of A (if present) is independently optionally substituted with at least one of C₁-C₆ alkyl, C₁-C₆ alkoxy, and halogen.

Embodiment 22: The compound of any one of Embodiment 20-21, wherein the compound is at least one selected from the group consisting of:

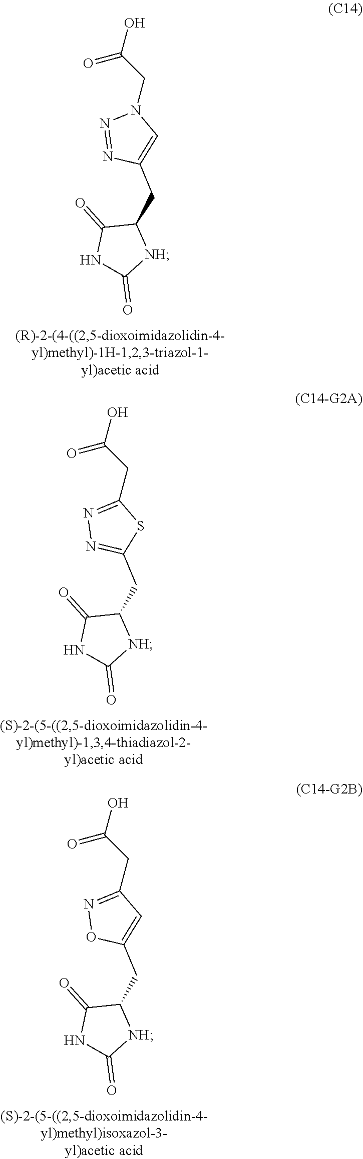 [SMILES: O=C(O)Cn1cc(C[C@H]2NC(=O)NC2=O)nn1 ; O=C(O)Cc1nnc(C[C@@H]2NC(=O)NC2=O)s1 ; O=C(O)Cc1cc(C[C@@H]2NC(=O)NC2=O)on1]

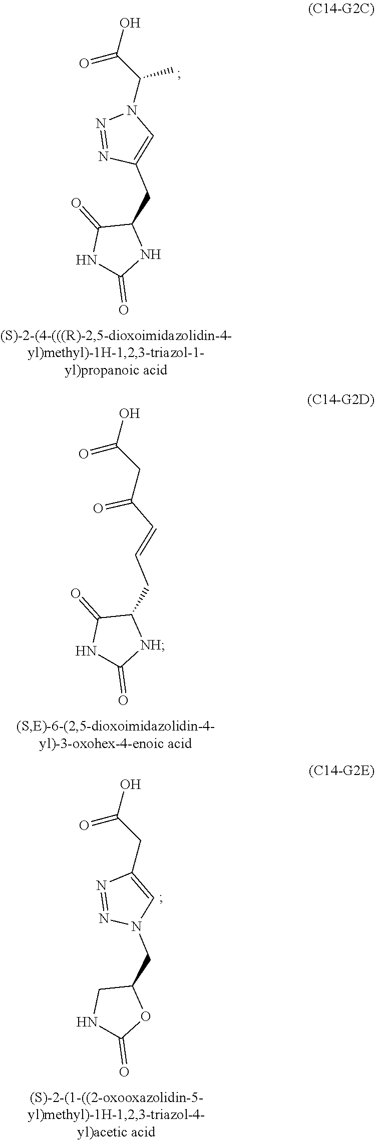 [SMILES: C[C@@H](C(=O)O)n1cc(C[C@H]2NC(=O)NC2=O)nn1 ; O=C(O)CC(=O)/C=C/C[C@@H]1NC(=O)NC1=O ; O=C(O)Cc1cn(C[C@@H]2CNC(=O)O2)nn1]

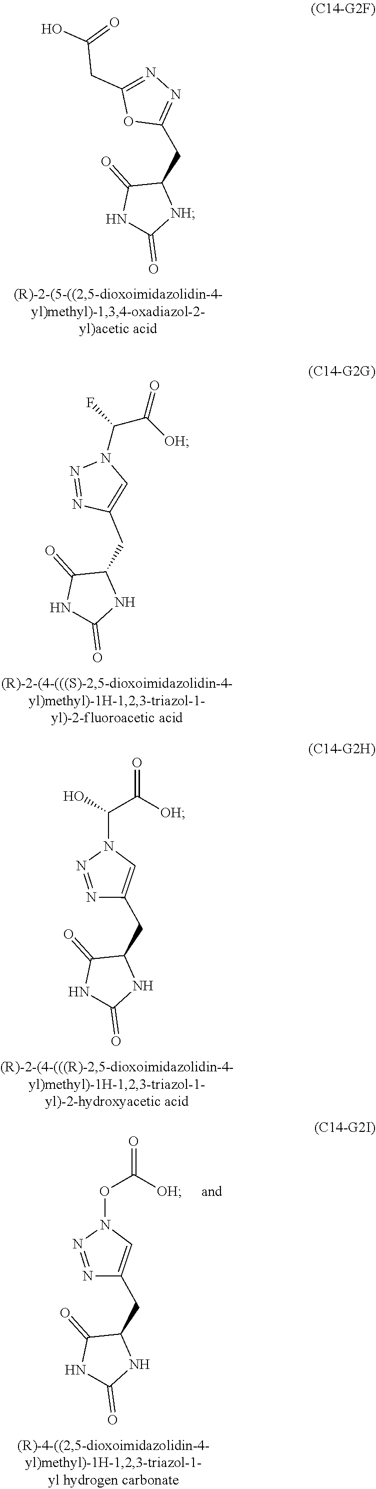 [SMILES: O=C(O)Cc1nnc(C[C@H]2NC(=O)NC2=O)o1 ; O=C1NC(=O)[C@H](Cc2cn([C@H](F)C(=O)O)nn2)N1 ; O=C1NC(=O)[C@@H](Cc2cn([C@H](O)C(=O)O)nn2)N1 ; O=C1NC(=O)[C@@H](Cc2cn(OC(=O)O)nn2)N1]

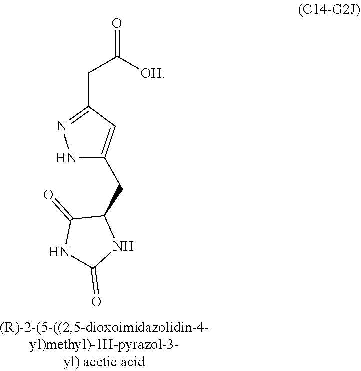 [SMILES: O=C(O)Cc1cc(C[C@H]2NC(=O)NC2=O)[nH]n1]

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

1. A method of treating, ameliorating, or preventing biofilm formation by a bacterium, the method comprising contacting the bacterium with at least one compound selected from
(a) a compound of Formula I: 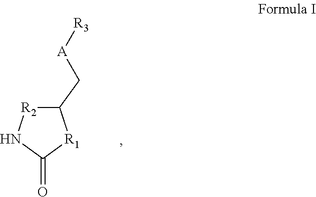
or a salt, solvate, tautomer, N-oxide, geometric isomer, stereoisomer thereof, or mixtures thereof, wherein:
R₁ is —NH— or —O—,
R₂ is —CH₂— or —C(O)—,
A is a five member aromatic heterocyclic ring or —CH═CH—COO—*, wherein * is the bond to R₃.
R₃ is —O—C(O)OH or 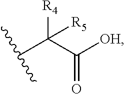
and
R₄ and R₅ are each independently H, halogen, C₁-C₆ alkyl, C₁-C₆ alkoxy, or —OH;
(b) 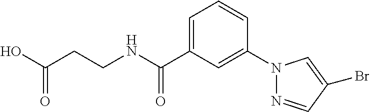 [SMILES: O=C(O)CCNC(=O)c1cccc(-n2cc(Br)cn2)c1]
3-(3-(4-bromo-1H-pyrazol-1-yl)benzamido)propanoic acid (C22), or a salt, solvate, tautomer, N-oxide, geometric isomer, or mixtures thereof.

2. The method of claim 1, wherein in Formula I, A is 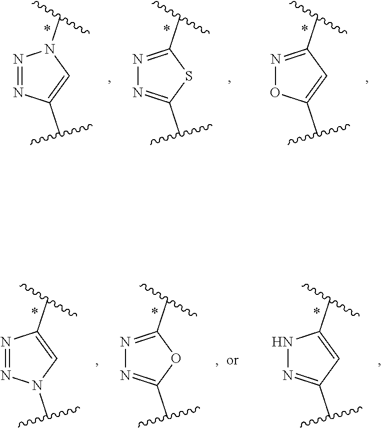 [SMILES: *c1cn(*)nn1 ; *c1nnc(*)s1 ; *c1cc(*)on1 ; *c1cn(*)nn1 ; *c1nnc(*)o1 ; *c1cc(*)[nH]n1]
wherein * is the bond to R₃ and wherein the CH in the five-membered heterocyclyl group of A (if present) is independently optionally substituted with at least one of C₁-C₆ alkyl, C₁-C₆ alkoxy, and halogen.

3. The method of claim 1, wherein the compound of Formula I is at least one selected from the group consisting of:

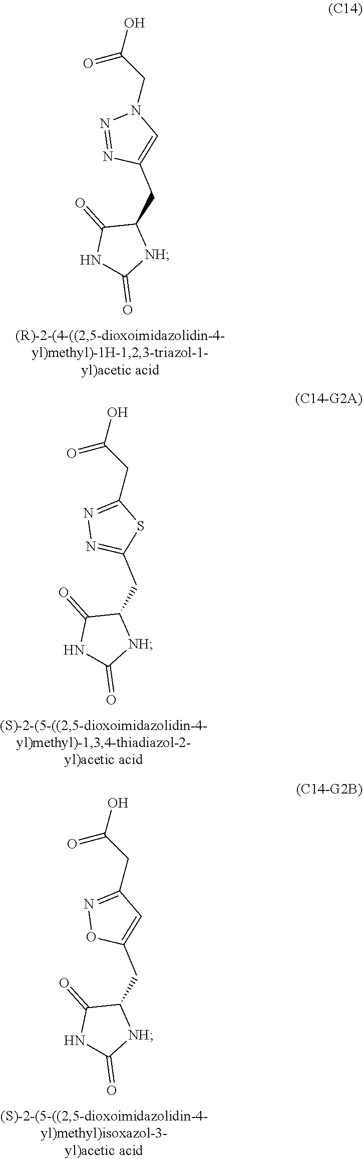 [SMILES: O=C(O)Cn1cc(C[C@H]2NC(=O)NC2=O)nn1 ; O=C(O)Cc1nnc(C[C@@H]2NC(=O)NC2=O)s1 ; O=C(O)Cc1cc(C[C@@H]2NC(=O)NC2=O)on1]

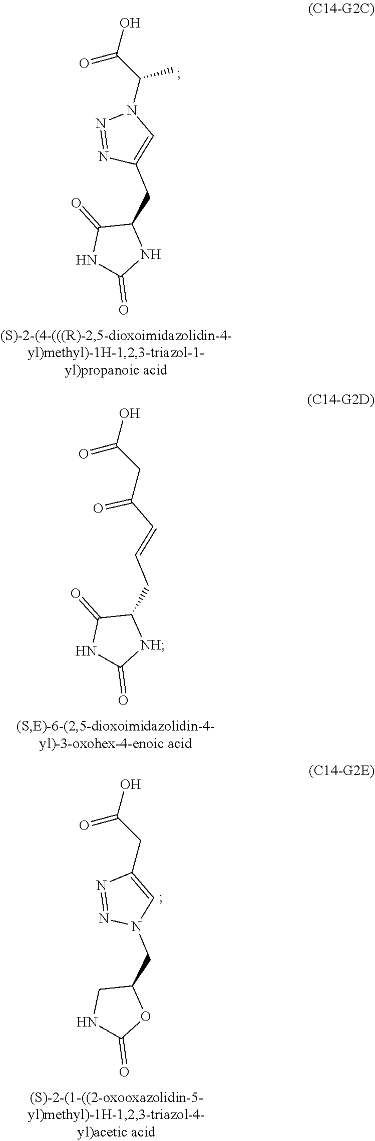 [SMILES: C[C@@H](C(=O)O)n1cc(C[C@H]2NC(=O)NC2=O)nn1 ; O=C(O)CC(=O)/C=C/C[C@@H]1NC(=O)NC1=O ; O=C(O)Cc1cn(C[C@@H]2CNC(=O)O2)nn1]

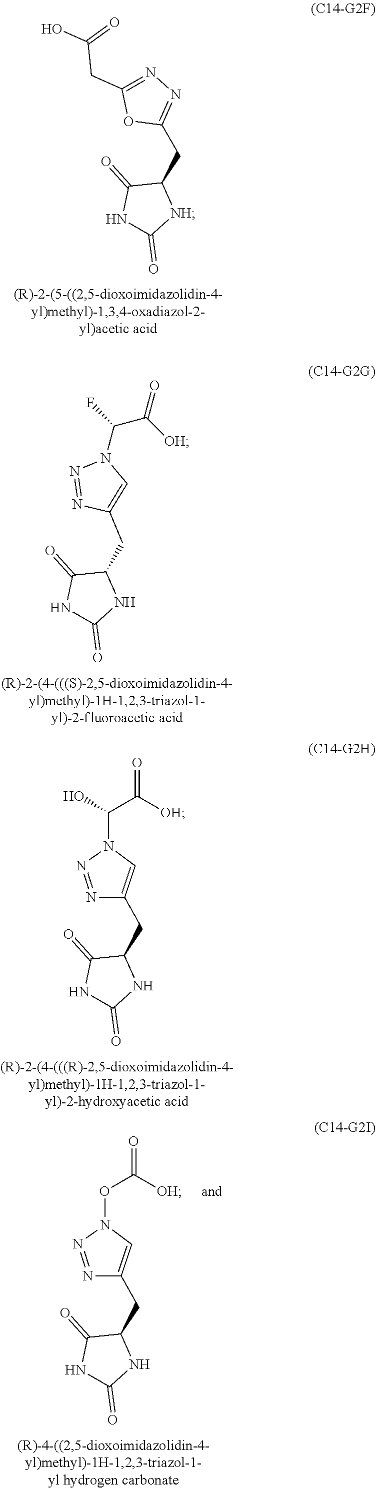 [SMILES: O=C(O)Cc1nnc(C[C@H]2NC(=O)NC2=O)o1 ; O=C1NC(=O)[C@H](Cc2cn([C@H](F)C(=O)O)nn2)N1 ; O=C1NC(=O)[C@@H](Cc2cn([C@H](O)C(=O)O)nn2)N1 ; O=C1NC(=O)[C@@H](Cc2cn(OC(=O)O)nn2)N1]

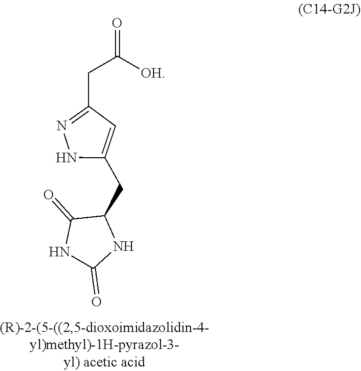 [SMILES: O=C(O)Cc1cc(C[C@H]2NC(=O)NC2=O)[nH]n1]

4. The method of claim 1, wherein formation of the biofilm by the bacterium is inhibited or wherein the integrity of the biofilm already formed by the bacterium is compromised or reduced.

5. (canceled)

6. The method of claim 1, wherein the bacterium is a gram-positive bacterium or a gram-negative bacterium.

7. The method of claim 1, wherein the bacterium comprises at least one of a B. burgdorferi bacterium, an E. coli bacterium, an H. influenzae bacterium, an N. gonorrhoeae bacterium, a P. aeruginosa bacterium, an S. epidermidis bacterium, an S. pneumoniae bacterium, and an S. aureus bacterium.

8. The method of claim 1, further comprises contacting the bacterium with an antibiotic for killing or inhibiting the bacterium.

9. The method of claim 1, wherein the biofilm is present in or on a subject, and the method comprises administering or applying an effective amount of the at least one compound to the subject.

10. The method of claim 9, wherein the biofilm is formed as part of a tissue-related infection in the subject or wherein the biofilm is formed in or on a device within the subject's body of the subject or in prolonged contact with the subject's body.

11. The method of claim 1, wherein the at least one compounds inhibits RelA or SpoT Homology (RSH) enzyme in the bacterium.

12. A method of inhibiting toxin production by a gram-negative bacterium, the method comprising contacting the gram-negative bacterium with at least one compound selected from:
(a) a compound of Formula I: 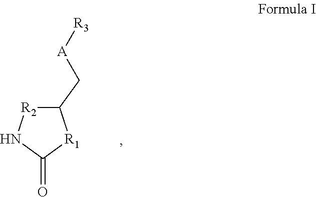
or a salt, solvate, tautomer, N-oxide, geometric isomer, stereoisomer thereof, or mixtures thereof, wherein:
R₁ is —NH— or —O—,
R₂ is —CH₂— or —C(O)—,
A is a five member aromatic heterocyclic ring or —CH═CH—COO—*, wherein * is the bond to R₃.
R₃ is —O—C(O)OH or 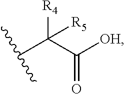
and
R₄ and R₅ are each independently H, halogen, C₁-C₆ alkyl, C₁-C₆ alkoxy, or —OH;
(b) 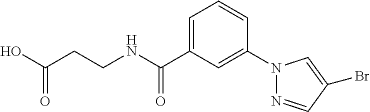 [SMILES: O=C(O)CCNC(=O)c1cccc(-n2cc(Br)cn2)c1]
3-(3-(4-bromo-1H-pyrazol-1-yl)benzamido)propanoic acid (C22), or a salt, solvate, tautomer, N-oxide, geometric isomer, or mixtures thereof.

13. The method of claim 12, wherein in Formula I, A is 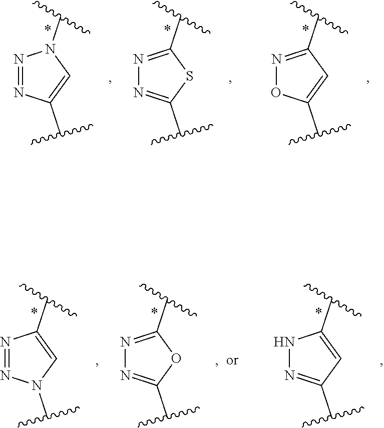 [SMILES: *c1cn(*)nn1 ; *c1nnc(*)s1 ; *c1cc(*)on1 ; *c1cn(*)nn1 ; *c1nnc(*)o1 ; *c1cc(*)[nH]n1]
wherein * is the bond to R₃ and wherein the CH in the five-membered heterocyclyl group of A (if present) is independently optionally substituted with at least one of C₁-C₆ alkyl, C₁-C₆ alkoxy, and halogen.

14. The method of claim 12, wherein the compound of Formula I is at least one selected from the group consisting of:

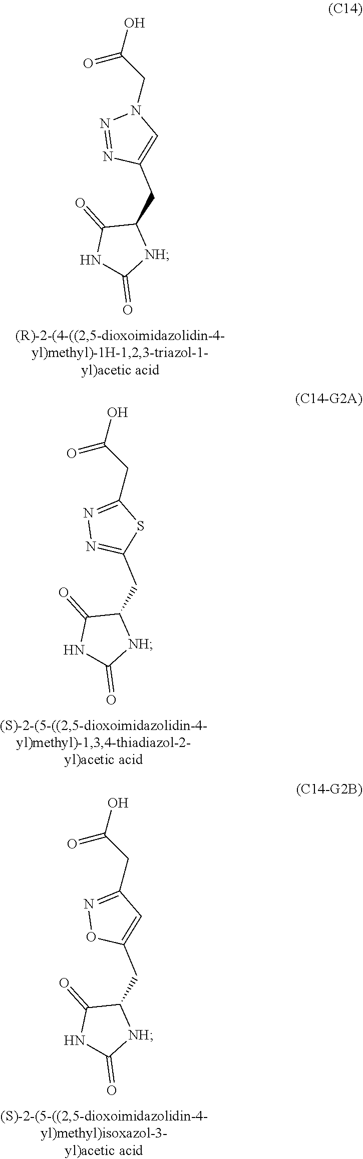 [SMILES: O=C(O)Cn1cc(C[C@H]2NC(=O)NC2=O)nn1 ; O=C(O)Cc1nnc(C[C@@H]2NC(=O)NC2=O)s1 ; O=C(O)Cc1cc(C[C@@H]2NC(=O)NC2=O)on1]

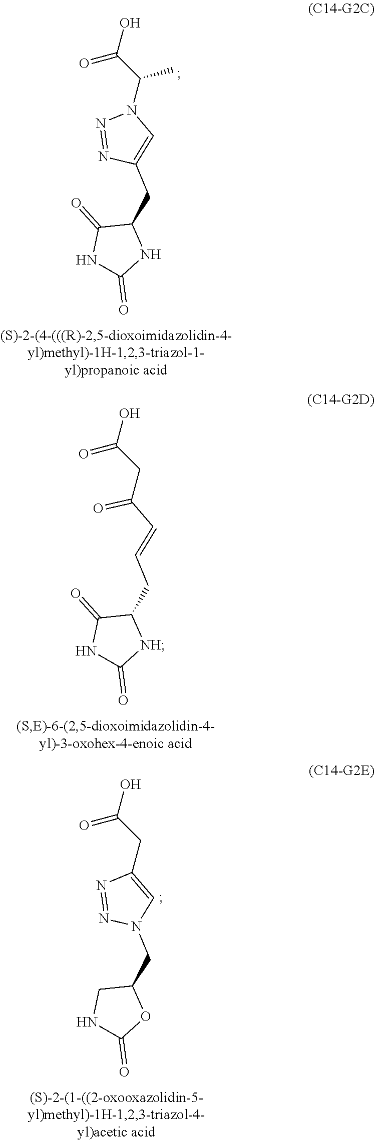 [SMILES: C[C@@H](C(=O)O)n1cc(C[C@H]2NC(=O)NC2=O)nn1 ; O=C(O)CC(=O)/C=C/C[C@@H]1NC(=O)NC1=O ; O=C(O)Cc1cn(C[C@@H]2CNC(=O)O2)nn1]

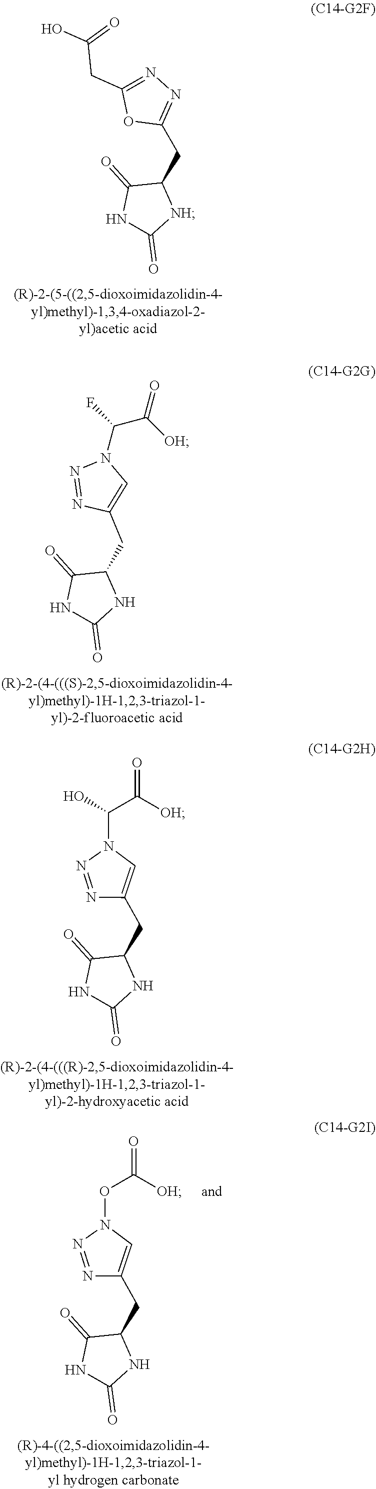 [SMILES: O=C(O)Cc1nnc(C[C@H]2NC(=O)NC2=O)o1 ; O=C1NC(=O)[C@H](Cc2cn([C@H](F)C(=O)O)nn2)N1 ; O=C1NC(=O)[C@@H](Cc2cn([C@H](O)C(=O)O)nn2)N1 ; O=C1NC(=O)[C@@H](Cc2cn(OC(=O)O)nn2)N1]

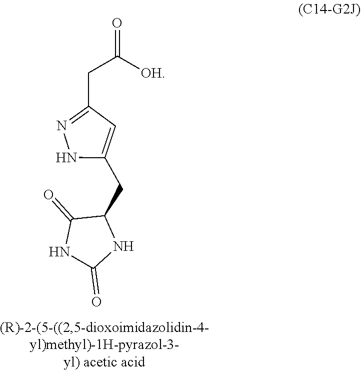 [SMILES: O=C(O)Cc1cc(C[C@H]2NC(=O)NC2=O)[nH]n1]

15. The method of claim 12, wherein the gram-negative bacterium comprises an E. coli bacterium, an H. influenzae bacterium, or a P. aeruginosa bacterium.

16. The method of claim 12, wherein the gram-negative bacterium is a cultured gram-negative bacterium or wherein the gram-negative bacterium is in or on the body of a subject.

17. (canceled)

18. The method of claim 16, wherein the gram-negative bacterium is in or on the body of a subject and wherein an effective amount of the at least one compound is administered or applied to the subject.

19. The method of claim 12, wherein RelA or RSH enzyme is inhibited in the gram-negative bacterium.

20. A compound of Formula I: 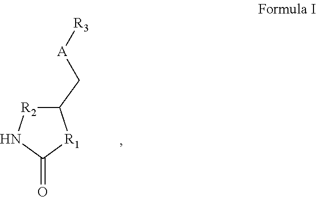
or a salt, solvate, tautomer, N-oxide, geometric isomer, stereoisomer thereof, or mixtures thereof,
wherein:
R₁ is —NH— or —O—,
R₂ is —CH₂— or —C(O)—,
A is a five member aromatic heterocyclic ring or —CH═CH—COO—*, wherein * is the bond to R₃.
R₃ is —O—C(O)OH or 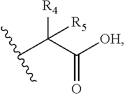
R₄ and R₅ are each independently H, halogen, C₁-C₆ alkyl, C₁-C₆ alkoxy, or —OH,
with the proviso that the compound is not 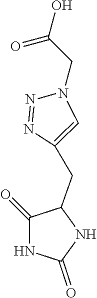 [SMILES: O=C(O)Cn1cc(CC2NC(=O)NC2=O)nn1]
2-(4-((2,5-dioxoimidazolidin-4-yl)methyl)-1H-1,2,3-triazol-1-yl)acetic acid.

21. The compound of claim 20, wherein in Formula I, A is 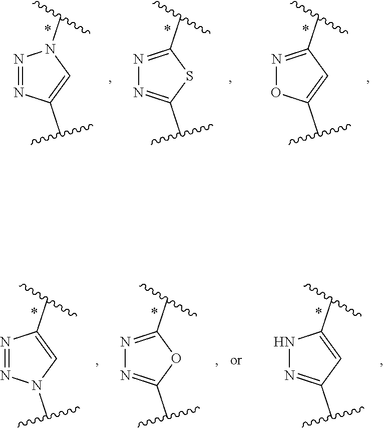 [SMILES: *c1cn(*)nn1 ; *c1nnc(*)s1 ; *c1cc(*)on1 ; *c1cn(*)nn1 ; *c1nnc(*)o1 ; *c1cc(*)[nH]n1]
wherein * is the bond to R₃ and wherein the CH in the five-membered heterocyclyl group of A (if present) is independently optionally substituted with at least one of C₁-C₆ alkyl, C₁-C₆ alkoxy, and halogen.

22. The compound of claim 20, wherein the compound is at least one selected from the group consisting of: 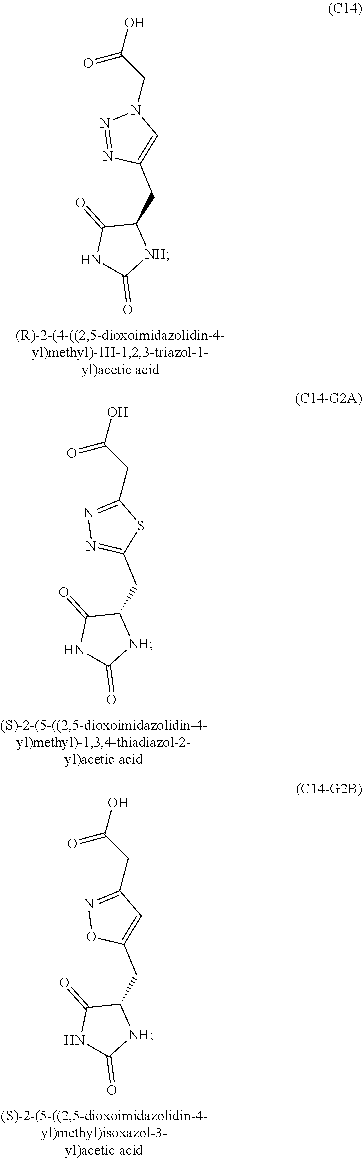 [SMILES: O=C(O)Cn1cc(C[C@H]2NC(=O)NC2=O)nn1 ; O=C(O)Cc1nnc(C[C@@H]2NC(=O)NC2=O)s1 ; O=C(O)Cc1cc(C[C@@H]2NC(=O)NC2=O)on1] 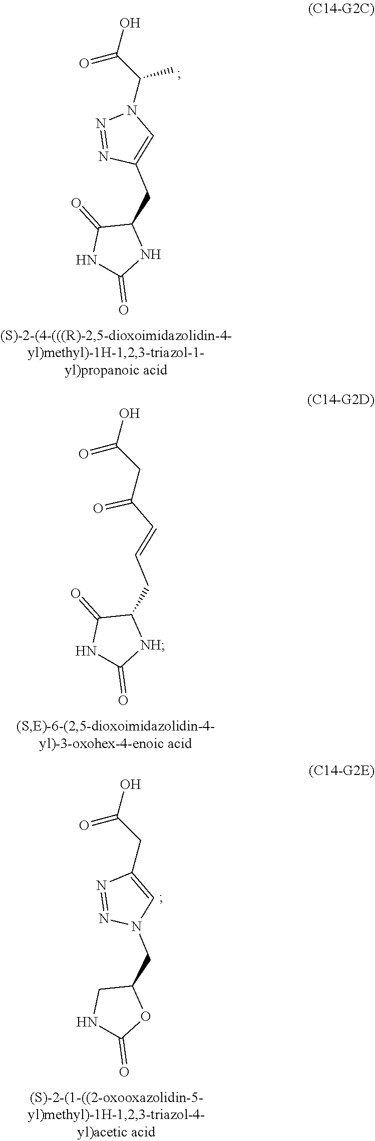 [SMILES: C[C@@H](C(=O)O)n1cc(C[C@H]2NC(=O)NC2=O)nn1 ; O=C(O)CC(=O)/C=C/C[C@@H]1NC(=O)NC1=O ; O=C(O)Cc1cn(C[C@@H]2CNC(=O)O2)nn1] 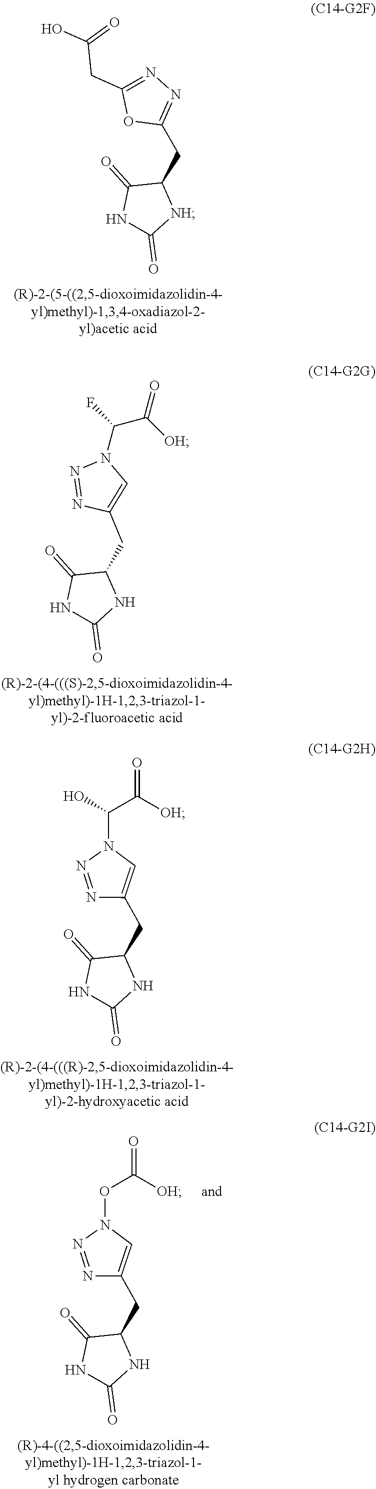 [SMILES: O=C(O)Cc1nnc(C[C@H]2NC(=O)NC2=O)o1 ; O=C1NC(=O)[C@H](Cc2cn([C@H](F)C(=O)O)nn2)N1 ; O=C1NC(=O)[C@@H](Cc2cn([C@H](O)C(=O)O)nn2)N1 ; O=C1NC(=O)[C@@H](Cc2cn(OC(=O)O)nn2)N1] 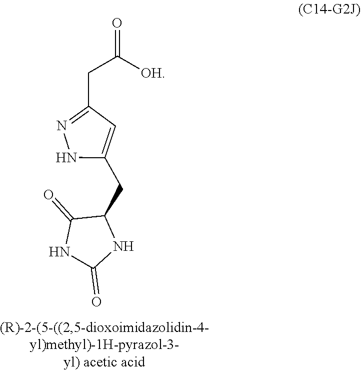 [SMILES: O=C(O)Cc1cc(C[C@H]2NC(=O)NC2=O)[nH]n1]


SEQUENCE LISTING (4 sequences)

SEQ ID NO: 1 - protein, 120 aa, Escherichia coli
  ACT_SITE 1..120: note = E. coli RelA amino acid residues 228-347
YIEEFVGHLRAEMKAEGVKAEVYGRPKHIYSIWRKMQKKNLAFDELFDVRAVRIVAERLQDCYAALGIVHTHYRHLPDEFDDYVANPKPNGYQSIHTVVLGPGGKTVEIQIRTKQMHEDA

SEQ ID NO: 2 - protein, 120 aa, Staphylococcus aureus
  ACT_SITE 1..120: note = S. aureus RelA amino acid residues 224-343
YIETAIDRIRTEMDRMNIEGDINGRPKHIYSIYRKMMKQKKQFDQIFDLLAIRVIVNSINDCYAILGLVHTLWKPMPGRFKDYIAMPKQNLYQSLHTTVVGPNGDPLEIQIRTFDMHEIA

SEQ ID NO: 3 - protein, 120 aa, Pseudomonas aeruginosa
  ACT_SITE 1..120: note = P. aeruginosa RelA amino acid residues 239-358
YIANVMSQLKEALAATGVQADLSGRAKHIYSIWRKMQRKGLDFSQIYDVRAVRVLVPEMRDCYTALGIVHTLWRHIPKEFDDYIANPKENGYRSLHTAVIGPEGKVLEVQIRTHSMHEEA

SEQ ID NO: 4 - protein, 119 aa, Streptococcus pneumoniae
  ACT_SITE 1..119: note = S. pneumoniae RelA amino acid residues 217-335
LVDEVVTKLEEYTTDRHLKGKIYGRPKHIYSIFRKMQDKRKRFEEIYDLIAIRCILDTQSDVYAMLGYVHELWKPMPGRFKDYIANRKANGYQSIHTTVYGPKGPIEFQIRTKAMHEVA